(19) United States
(12) Patent Application Publication
(10) Pub. No.: US 2003/0175976 A1
(43) Pub. Date: Sep. 18, 2003

(54) Clean synthetic vectors, plasmids, transgenic plants and plant parts containing them, and methods for obtaining them

(76) Inventors: Veronique Gruber, Romagnat (FR); David Comeau, LaRoche Blanche (FR)

Correspondence Address: PALMER & DODGE, LLP, KATHLEEN M. WILLIAMS, 111 HUNTINGTON AVENUE, BOSTON, MA, 02199, US

(21) Appl. No.: 09/845,064

(22) Filed: Apr. 27, 2001

(30) Foreign Application Priority Data
Sep. 4, 2000 (WO) PCT/IB00/01243
Sep. 3, 1999 (FR) 99111112

(51) Int. Cl. C12N 15/82; C12N 15/00

(52) U.S. Cl. 435/468; 435/320.1

(57) ABSTRACT

The present invention relates to clean synthetic vectors intended to facilitate and improve the insertion of one or more genes of interest into a eukaryotic cell, and notably into a plant cell. The invention also relates to a procedure for obtaining these vectors as well as transgenic plants containing them.

This application claims the priority of PCT Application No. PCT/IB00/01243, filed Sep. 4, 2000, and published in English on Mar. 15, 2001, which application claims the priority of French Patent Application No. 99111112, filed Sep. 3, 1999.

The present invention relates to clean synthetic vectors, intended notably for use for genetic transformation in the field of plant biotechnology.

In general, the vectors are known in the field of biotechnology and genetic manipulation. The vectors which are currently used most commonly for genetic transformation, and in particular in the field of plant biotechnology, present several disadvantages in their use, however. In actual fact, and notably for plant transgenesis, use has often been made of a vector called pBin19 (Frisch et al., 1995). The nucleotide sequence of this binary plasmid pBin19 is entirely known. The problem, however, is that this plasmid is of large size (11.8 kbp) and that it contains useless elements (more than half of pBin19) which are intolerable from a regulatory point of view, or even detrimental to good replication. Moreover, the selection cassette of this plasmid is located near the right border of the T-DNA. Thus, when the T-DNA is broken after the selection cassette, it will not be possible to make any selection in order to retain solely the plants possessing the expression cassette of the desired gene.

The expressions used in the description and the claims have the following meaning:

“vector” means an expression system, for example DNA-coated projectiles, nucleic-acid-based transit vehicles, nucleic acid molecules adapted to deliver nucleic acid, and autonomous self-replicating circular DNA, for example plasmids, cosmids, phagemids, etc. If a micro-organism or a recombinant cell culture is described as host of an “expression vector”, this can also include extrachromosomal circular DNA (such as for example mitochondrial or chloroplast DNA), DNA which has been integrated into the host chromosome(s), where the vector can be either replicated in a stable manner by the cells during mitosis as an autonomous structure, integrated into the genome of the host, or maintained in the nucleus or cytoplasm of the host.

usually the vectors used for the genetic transformation exist in the form of plasmids. In this case, the “plasmid” is a molecule of autonomous circular DNA capable of replication in a cell. If a micro-organism or recombinant cell culture is described as the host of an “expression” plasmid, this comprises both extrachromosomal circular DNA molecules and DNA which has been integrated into the host chromosome(s). If the plasmid is maintained by a host cell, the plasmid is either replicated in a stable manner by the cells during mitosis as an autonomous structure, or integrated into the genome of the host;

“clean” means that the vector comprises only sequences that are indispensable for its functionality and carries a nucleic acid sequence which comprises only elements that are indispensable for the expression of the host cell;

“nucleic acid” means DNA or RNA;

“nucleic acid sequence” means a single- or double-stranded oligomer or polymer of nucleotide bases read from the 5′ end towards the 3′ end, and comprises self-replicating plasmids, genes, DNA or RNA polymers, which may or may not be infectious, and DNA, or RNA, either functional or non-functional. In the nucleotide notation used in the present application, unless specifically stated, the left end of a single-stranded nucleotide sequence is the 5′ end;

“derived nucleic acid sequence” means that the sequence derives directly or indirectly from the sequence referred to, for example by substitution, deletion, addition, mutation, fragmentation, and/or synthesis of one or more nucleotides;

“promoter” means a nucleic acid region which is upstream of the translation initiation codon and which is involved in the recognition and binding of RNA polymerase and other transcription proteins;

“plant promoter” is a promoter capable of initiating transcription in plant cells;

“constitutive promoter” is a promoter capable of expressing nucleic acid sequences operationally bound to said promoter, in all or practically all the tissues of the host organism, during the whole development of said organism;

“tissue specific promoter” is a promoter capable of selectively expressing nucleic acid sequences operationally bound to said promoter, in certain specific tissues of the host organism;

“operationally bound” means the binding of a functional or regulatory element, for example a promoter, to the nucleic acid sequence, or gene, to be expressed which codes for a protein to be produced, in such a way that this element influences the transcription of the bound nucleic acid sequence;

“expression cassette” means nucleotide sequences capable of directing the expression of a nucleic acid sequence, or of a gene, coding for a polypeptide to be produced in a host organism compatible with such sequences. Such cassettes include at least one promoter and a transcription termination signal, and optionally other factors necessary or useful for the expression;

“heterologous sequence” or “heterologous nucleic acid sequence” means a sequence originating from a source, or from a species, foreign to the environment thereof, or if it originates from the same environment, which has been modified relative to its original form. The modification of the nucleic acid sequence can take place for example by treatment of the nucleic acid with a restriction enzyme in order to generate a nucleic acid fragment which can be operationally bound to a promoter. The modification can also take place by means of techniques such as directed mutagenesis;

“box” means a nucleic acid sequence to which a regulatory function is attributed;

“like” means that the box, and/or the nucleic acid sequence with which this term is associated, involves a certain sequence identity or consensus with a box and/or a known nucleic acid sequence, called a reference sequence, preferably a sequence identity of at least 50%, more preferably a sequence identity of at least 75%, and more particularly a sequence identity of at least 90% with the reference sequence. The percentage of sequence identity is calculated on the basis of a comparison window of at least 6 nucleotide bases. The determination of a comparison window can be carried out using sequence alignment algorithms to determine a homology with a reference sequence, for example the local homology algorithm, the homology alignment algorithm, and the similarity search algorithm, these algorithms existing also in computerized form, known by the names GAP, BESTFIT, FASTA and TFASTA. The percentage of sequence identity is obtained by comparing the reference sequence with the box and/or the nucleic acid sequence;

“situated” means the position on a nucleic acid sequence of an identified element, such as a “box”, a restriction site, or a codon having a particular function. The position which is given by a figure refers to the position of the start of the element in the nucleic acid sequence, in the direction of reading of the latter, i.e. in the direction 5′→3′;

“transgenic plant” means a plant which has been obtained by genetic manipulation techniques, and covers the whole plants obtained, their progeny, and the plant organs, for example the roots, stems and leaves, obtained by these techniques. The transgenic plants according to the present invention can have different levels of ploidy, and can notably be polyploid, diploid, and haploid;

“propagule” means a cluster or association of plant cells, which may or may not be structured, allowing the regeneration of a whole plant, for example explants, calli, stems, leaves, roots, cuttings, and even seeds.

The applicant of the present invention has succeeded, surprisingly, in producing clean synthetic vectors, in particular binary plasmids, of completely known nucleotide sequence, of small size, allowing the aforesaid disadvantages to be alleviated, and notably presenting a high replication rate relative to the existing vectors most commonly used. Furthermore, the applicant has succeeded at the same time in producing a range of vectors in such a way as to be able to choose the one which it is convenient to use according to the application envisaged and the environment of its use, and thus in such a way as to be able to better control the rate of expression of a gene to be expressed, coding for a polypeptide to be produced.

In addition, in some of the vectors according to the invention, each of the functional elements or components can be isolated by simple enzymatic digestion.

An object of the present invention is therefore a clean synthetic vector containing only the elements indispensable to its functionality and to the transgenesis of a cell, and notably of a plant cell.

According to a preferred embodiment, the vector comprises, as elements which are indispensable to its functionality and to the transgenesis of a cell, and which are operationally bound:

at least one nucleic acid sequence coding for at least one first origin of replication, preferably an ori RK2, and more preferably at least one ori V of pRK2 of Escherichia coli with a broad host range;

at least one nucleic acid sequence coding for a selection agent, preferably an antibiotic resistance gene, more preferably the npt III gene conferring resistance to kanamycin in bacteria;

a trfA locus coding for at least one protein allowing an increase in the replication rate of the plasmid, preferably originating from pRK2, and more preferably coding for the proteins P285 and P382.

According to a more preferred embodiment the vector comprises the nucleic acid sequence identified by the number SEQ.ID01. Still more preferably, the vector consists of a single plasmid pMRT1105 whose nucleic acid sequence is identified by the number SEQ.ID01.

According to another preferred embodiment, the vector includes at least one nucleic acid sequence coding for a second origin of replication, preferably an ori of Escherichia coli, and more preferably an ori ColEI. More preferably, the vector comprises the nucleic acid sequence identified by the number SEQ.ID02, and still more preferably, the vector consists of a single plasmid pMRT1106 whose nucleic acid sequence is identified by the number SEQ.ID02.

Preferably, the synthetic vector according to the invention comprises a region (200 bp or less) made up of at least one nucleic acid sequence containing several unique enzymatic restriction sites called collectively <<multiple cloning sites>> (MCS).

According to a preferred embodiment, the vector also includes a nucleic acid sequence coding for a T-DNA comprising a right border RB and a left border LB, allowing the vector to act as a binary plasmid.

Preferably, the MCS is situated near (within 300 bp of) the right border, RB, of the T-DNA prior to the cloning of an insert into the MCS.

According to another preferred embodiment, the vector also includes a nucleic acid sequence coding for at least one expression promoter and at least one transcription terminator situated between the left border LB and the right border RB of the T-DNA. More preferably, the expression promoter is chosen from the group consisting of the constitutive promoters, the inducible promoters, the specific promoters, and preferably chosen from the plant expression promoters. Still more preferably, the expression promoter is chosen from the group consisting of the 35S CaMV promoter, the ep35S of CaMV, the pea plastocyanin gene promoter, its “enhancer” zones and derivatives, the wheat “High Molecular Weight Glutenin” (HMWG) promoter, the “Cassava Vein Mosaic Virus” CsVMV promoter, the “Commelina Yellow Mottle Virus” CoYMV promoter, the chimeric promoters of the CsVMV and CoYMV promoters, and their derivatives.

Preferably, the expression terminator is chosen from the functional terminators in a plant cell, and is preferably a 35S or nos terminator.

According to yet another preferred embodiment, the vector comprises at least one nucleic acid sequence coding for a selection agent that is functional in a plant cell, preferably at least one acid sequence coding for an antibiotic resistance gene, and/or a herbicide resistance gene.

Preferably, the sequence coding for a selection agent is a sequence coding for the bar (<<bialaphos resistance>>) resistance gene or pat (<<phosphinothricin acetyltransferase>>) gene, or else a sequence coding for the mutant or wild-type resistance gene nptII. More preferably still, the nucleic acid sequence coding for the selection agent is situated near the left border of the T-DNA.

According to another preferred embodiment of the present invention, the vector includes at least one expression cassette comprising an expression-promoting nucleic acid sequence operationally bound to a nucleic acid sequence to be expressed, coding for a polypeptide to be produced, itself bound to a transcription termination nucleic acid sequence. Preferably, the polypeptide to be produced is an enzyme or protein or derivative of the latter having activity in vitro and/or in man and/or in animals, said activity comprising digestive, pancreatic, biliary, antiviral, anti-inflammatory, pulmonary, anti-microbial, nutritional, cosmetic, structural, blood, cardiovascular, ophthalmic, antigenic, immunostimulatory and cerebral activity. Examples of such proteins are for example the insulins, interferons, gastric, pancreatic or biliary lipases, elastases, antiproteases such as alpha-1 antitrypsin, structural proteins such as collagen, transferrins such as lactoferrin, proteins derived from blood, such as haemoglobin, human albumin and the blood cofactors, and antioxidants such as superoxide dismutase,

According to a particularly preferred embodiment of the invention, the vector is presented in the form of a binary, linear or circular plasmid, chosen from the group consisting of the nucleic acid sequences identified by the numbers SEQ.ID03, SEQ.IDO4, SEQ.ID05, SEQ.ID06, SEQ.ID07, SEQ.ID08, SEQ.IDO9, SEQ.ID10, SEQ.ID11, SEQ.ID12, SEQ.ID13, SEQ.ID14, SEQ.ID15, SEQ.ID16, SEQ.ID17, SEQ.ID18, SEQ.ID19, SEQ.ID20, SEQ.ID21 and SEQ.ID22.

According to a particularly advantageous embodiment, each functional component of the vector can be cleaved independently of the other components. Preferably, each functional component can be cleaved independently of the other components by enzymatic digestion at the level of a first unique restriction site and a second unique restriction site which are present in 1 vector.

Another object of the present invention is an isolated nucleic acid sequence, characterized in that it corresponds to a nucleic acid sequence chosen from the group consisting of the nucleic acid sequences identified by the numbers SEQ.ID01, SEQ.ID02, SEQ.ID03, SEQ.IDO4, SEQ.ID05, SEQ.ID06, SEQ.ID07, SEQ.ID08, SEQ.IDO9, SEQ.ID10, SEQ.ID11, SEQ.ID12, SEQ.ID13, SEQ.ID14, SEQ.ID15, SEQ.ID16, SEQ.ID17, SEQ.ID18, SEQ.ID19, SEQ.ID20, SEQ.ID21 and SEQ.ID22.

Yet another object of the present invention is a cell containing a vector or a nucleic acid sequence such as described earlier. The cell is preferably a plant cell.

Yet another object of the present invention is a transgenic plant having stably integrated in its genome a vector or a nucleic acid sequence such as described earlier. The plant is preferably chosen from the dicotyledon species, such as potato, tobacco, cotton, lettuce, tomato, melon, cucumber, pea, rape, beetroot or sunflower, or the monocotyledon species, such as wheat, barley, oats, rice or maize.

Yet another object of the present invention is a propagule of a transgenic plant such as described earlier. The propagule is preferably a seed.

Yet another object of the present invention is a method for expression of a nucleic acid sequence, or gene, coding for a polypeptide to be produced, in a cell, characterized in that it comprises the stages consisting of:

transforming the cell with a vector or a nucleic acid sequence such as described earlier;

making a culture of the cell under conditions allowing the expression of a nucleic acid sequence, or gene, coding for the polypeptide to be produced. The cell is preferably a prokaryotic or eukaryotic cell. More preferably, the cell is a cell chosen from the group consisting of microbial cells, fungal cells, insect cells, animal cells and plant cells. Still more preferably, the cell is a plant cell.

Yet another object according to the present invention is a method for obtaining a transgenic plant or a propagule such as described earlier, characterized in that it comprises the stages consisting of:

transforming a plant cell with a vector comprising or a nucleic acid sequence according to any one of claims 1 to 24;

selecting the plant cell having the vector or nucleic acid sequence integrated therein;

propagating the transformed and selected plant cell, either by culture or by regeneration of whole chimeric or transgenic plants.

Thus, the new synthetic vectors, and preferably, binary plasmids, according to the invention have only regions indispensable for the functionality of the vector and for transgenesis. The applicant has found that they have a high replication rate. In addition, they can have a selection cassette near the left border, as well as rare restriction sites in their <<polylinkers>> (multiple cloning sites).

### DESCRIPTION OF THE FIGURES

The invention will be understood better from the detailed description of the different embodiments which is given below in the form of examples, which are not limiting, and with reference to the drawing in the annex, in which:

FIG. 1 schematically represents a preferred minimal vector according to the present invention in the form of a plasmid identified by the reference pMRT1105, and with a length of 3508 base pairs;

FIG. 2 represents a schematic chart of a variant of the vector of FIG. 1 in the form of a plasmid identified by the reference pMRT1106, and with a length of 4098 base pairs;

FIG. 3 represents a schematic chart of a preferred vector according to the present invention, in the form of a binary plasmid identified by the reference pMRT1118, with a length of 5971 base pairs, and including a T-DNA comprising a selection cassette coding for resistance to an antibiotic, and a multiple cloning site (MCS);

FIG. 4 represents a preferred embodiment of the vector according to FIG. 3, the multiple cloning site (MCS) including yet other unique sites, the vector being in the form of a binary plasmid identified by the reference pMRT1119, and with a length of 6016 base pairs;

FIGS. 5 to 22 represent other preferred embodiments of the vector according to the invention, in the form of binary plasmids identified respectively by the references pMRT1121 (6017 base pairs), pMRT1122 (6016 base pairs), pMRT1155 (6017 base pairs), pMRT1175 (6767 base pairs), pMRT1176 (6767 base pairs), pMRT1191 (4805 base pairs), pMRT1192 (8654 base pairs), pMRT1193 (9143 base pairs), pMRT1195 (6865 base pairs), pMRT1196 (8654 base pairs), pMRT1201 (7943 base pairs), pMRT1202 (5614 base pairs), pMRT1203 (7503 base pairs), pMRT1204 (9390 base pairs), pMRT1205 (7503 base pairs), pMRT1206 (9390 base pairs), pMRT1210 (10003 base pairs) and pMRT1212 (8987 base pairs);

FIG. 23 graphically represents a comparison between the protein expression capacities of the synthetic vectors according to the present invention in comparison to a known vector system;

FIGS. 24 to 28 represent further embodiments of vectors according to the present invention, in the form of binary plasmids identified by the references pMRT1334 (9688 bp), pMRT1335 (15208 bp), pMRT1336 (9285 bp), pMRT1337 (8289 bp), pMRT1341 (14108 bp), pMRT1342 (15077 bp).

In the figures, the abbreviations have the following meanings:

ori RK2: ori V of pRK2 of Escherichia coli with a broad host range, unstable RK2 replicon producing a low number of copies;

ori ColEI: the origin of replication ColE1 v of Escherichia coli;

npt III gene: coding for neomycin phosphotransferase conferring resistance to kanamycin

npt II gene: coding for neomycin phosphotransferase conferring resistance to kanamycin

trfA: trfA locus (1481 bp) originating from pRK2 allowing the production of 2 proteins, P285 and P382, which enhance the replication of the plasmid by binding to the origin of replication;

Tnos: nos (nopaline synthetase) terminator;

Pnos: nos (nopaline synthetase) promoter;

LB: left border of a T-DNA;

RB: right border of a T-DNA;

MCS: multiple cloning site, S:XXX indicating the number of the starting base, E:XXX indicating the number of the end base of the MCS, the list starting with the first restriction site at the top and ending with the last restriction site at the bottom, in the direction 5′>3′;

polyA 35S: transcription termination (polyadenylation) signal;

gus gene: gene coding for beta-glucuronidase;

IA: the actin intron of rice;

PA: actin promoter of rice;

Phmwg: <<high molecular weight glutenin>> promoter of wheat;

bar (<<bialaphos resistance>>) gene: gene coding for the enzyme phosphinothricin acetyltransferase conferring resistance to glufosinate;

ep35S: <<enhanced promoter>> of the 35S ribosome.

### EXAMPLES

In the detailed description which follows, all the enzymatic digestions by restriction enzymes were performed in accordance with the recommendations of the supplier New England Biolabs. The purifications with the aid of the <<QIAquick Gel Extraction>> and <<QIAquick PCR Purification>> kits were carried out in accordance with the recommendations of the supplier QIAGEN. The <<Concert Rapid PCR Purification System>> kit was used in accordance with the instructions of the supplier GIBCO BRL Life Technologies. The <<GeneAmp PCR System 9700>> thermocycler used is marketed by Perkin Elmer Applied Biosystems.

### Example 1

1. Synthesis of pMRT1105

A clean synthetic vector according to the invention is presented preferably in the form of a minimal plasmid pMRT1105 (3508 bp) and is made up of the following elements:

ori RK2: ori V of pRK2 of Escherichia coli with a broad host range, unstable RK2 replicon producing a small number of copies (643 bp).

npt III gene: confers resistance to kanamycin (bacterial selection marker, 1337 bp).

TrfA: trfA locus (1481 bp) originating from pRK2, allowing the production of 2 proteins, P285 and P382, which enhance the replication of the plasmid by binding to the origin of replication.

The plasmid pMRT1105 results from the assembling of the fragments obtained by splicing overlap extension for <<ori RK2>> and a <<part of npt III>>, of the fragment corresponding to a <<part of trfA>> produced by PCR (<<polymerase chain reaction>>) amplification and of <<parts of trfA and npt III>> isolated by enzymatic digestion.

1.1. Synthesis of the Fragment Carrying <<ori RK2 and Part of npt III>>

The AvrII-StuI fragment (654 bp) carrying <<ori RK2>> (643 bp) was amplified by PCR from 5 ng of pBin19 matrix DNA with the aid of 20 pmoles of each of the 2 oligodeoxynucleotides, 5′ AACCTAGGAAAAGACCGAGCGCCTTTGC 3′ (SEQ.ID23) containing the AvrII restriction site and 5′ CGGATTAATGGTAGAAGGCCTTTCACGGGAGGGTTCGAGAAGG 3′ (SEQ.ID24) possessing the StuI restriction site, in the presence of 200 μM of each of the dNTPs, 60 mM Tris-SO₄ pH 9.1, 18 mM (NH₄)₂ SO₄, 1.8 mM MgSO₄ and 2 U of ELONGASE enzyme (GIBCO BRL Life Technologies) in a final reaction medium of 50 μl. The PCR amplification reaction was carried out in the <<GeneAmp PCR System 9700>> thermocycler. After denaturation at 94° C. for 3 min., the DNA was subjected to 15 cycles each made up of the stages of denaturation at 94° C. for 30 sec., hybridization at 55° C. for 30 sec. and elongation at 68° C. for 45 sec. Then, in the last cycle, the elongation was continued at 68° C. for 3 min. Forty μl of the PCR reaction medium were then subjected to the action of 12.5 U of the Klenow fragment (New England Biolabs) in the presence of 2 μl of each of the dNTPs at 10 mM. The reaction was carried out at 37° C. for 10 min. The PCR product so treated was then isolated by 2% agarose gel electrophoresis in TBE buffer (90 mM Tris-HCl, 2 mM Na₂-EDTA, 90 mM boric acid, pH 8.0) and purified with the aid of the <<QIAquick Gel Extraction>> kit. The DNA was recovered in 30 μl of H₂O.

The StuI-BstXI fragment (363 bp) carrying <<part of npt III>> (344 bp) was amplified and treated in the same way as the <<ori RK2>> fragment except that the 2 oligodeoxynucleotides used are 5′ TGAAAGGCCTTCTACCATTAATCCGCGATAAACCCAGCGAACC 3′ (SEQ.ID25) containing an StuI restriction site and 5′ ATGCATCCAAAATTTTGGTAGAATTTACAAGCTATAAGGTTATTGTCCTGGG 3′ (SEQ.ID26) possessing the BstXI restriction site.

The 2 fragments carrying <<ori RK2>> and <<part of npt III>> were then assembled by splicing overlap extension in order to obtain the fragment <<ori RK2 and part of npt III>>. To do this, a PCR amplification was performed from 7.5 μl of each of the treated PCR products corresponding to <<ori RK2>> and <<part of npt III>> with the aid of 20 pmoles of each of the oligodeoxynucleotide 5′

(SEQ.ID23)

5′ AACCTAGGAAAAGACCGAGCGCCTTTGC 3′ and

(SEQ.ID26)

5′ ATGCATCCAAAATTTTGGTAGAATTTACAAGCTATAAGGTTATTGTC

CTGGG 3′

in the presence of 200 μM of each of the dNTPs, 60 mM Tris-SO₄ pH 9.1, 18 mM (NH₄)₂ SO₄, 1.8 mM MgSO₄ and 2 U of ELONGASE enzyme in a final reaction medium of 50 μl. The PCR amplification reaction was carried out in the <<GeneAmp PCR System 9700>> thermocycler. After denaturation at 94° C. for 3 min., the DNA was subjected to 15 cycles each made up of the stages of denaturation at 94° C. for 30 sec., hybridization at 62° C. for 30 sec. and elongation at 68° C. for 45 sec. Then, in the last cycle, the elongation was continued at 68° C. for 3 min. Forty μl of the PCR reaction medium were then subjected to 1.5% agarose gel electrophoresis in TBE buffer and purified with the aid of the <<QIAquick Gel Extraction>> kit. The DNA, corresponding to the fragment carrying <<ori RK2>> and <<part of npt III>>, was recovered in 30 μl of H2O. Next, 27 μl of this DNA were hydrolysed by BstXI followed by AvrII, then purified with the aid of the <<QIAquick PCR Purification>> kit and recovered in 30 μl of H₂O.

1.2. Synthesis of the Fragment Carrying a <<Part of TrfA>>

The NdeI-AvrII fragment (295 bp) carrying <<part of trfA>> (295 bp) was amplified and treated in the same way as the fragment <<ori RK2>> except that the 2 oligodeoxynucleotides used are 5′ ATCGACGAGGAAATCGTCGTGCTGTTTGC 3′ (SEQ.ID27) situated upstream of the NdeI site and 5′ AAACCTAGGAAATGCCAGTAAAGCGCTGGC 3′ (SEQ.ID28) possessing the AvrII restriction site. The DNA fragments corresponding to a <<part of trfA>> originating from the PCR amplification are then purified with the aid of the <<QIAquick PCR Purification>> kit, recovered in 30 μl of H2O, hydrolysed by AvrII and NdeI, repurified with the aid of the <<QIAquick PCR Purification>> kit and recovered in 30 μl of H₂O.

1.3. Synthesis of the Fragment Carrying the <<Parts of TrfA and npt III>>

The BstXI-NdeI fragment (2196 bp) carrying the <<parts of trfA and npt III>> (2196 bp) was isolated from 9 μg of pBin19 DNA by enzymatic digestion by BstXI, purified with the aid of the <<QIAquick PCR Purification>> kit, then hydrolysed by NdeI. The DNA fragment was then isolated by 0.9% agarose gel electrophoresis in TBE buffer, purified with the aid of the <<QIAquick Gel Extraction>> kit and recovered in 30 μl of H2O.

1.4. Obtaining pMRT1105

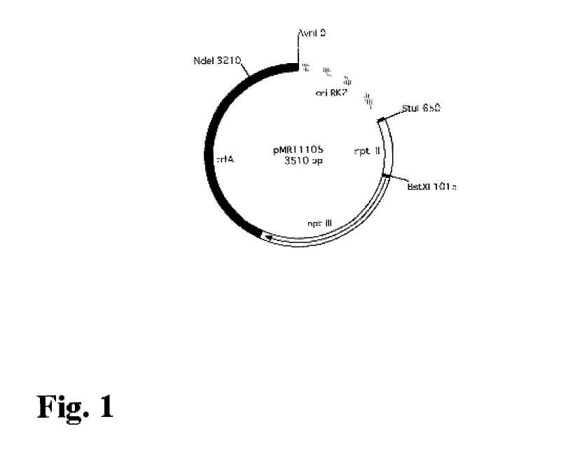

The 3 fragments, <<ori RK2 and part of npt III>>, <<parts of trfA and npt III>> and <<part of trfA>>, were ligated. To do this, the reaction mixture containing 7.5 μl of the digested fragments corresponding to <<ori RK2 and part of npt III>> and <<parts of trfA and npt III>>, and 11 μl of the digested fragment corresponding to <<part of trfA>>, was concentrated with the SpeedVac AES1000 (SAVANT) in order to achieve a volume of 8 μl. After that, 1 μl of T4 DNA ligase×10 buffer (New England Biolabs) and 400 units of T4 DNA ligase enzyme (New England Biolabs) were added. The ligation was carried out by a PCR reaction made up of 200 cycles each consisting of 2 stages, one at 30° C. for 30 sec. and the other at 10° C. for 30 sec., in the <<GeneAmp PCR System 9700>> thermocycler. The bacteria, Escherichia coli DH5, which had previously been made competent, were transformed (Hanahan, 1983). The plasmid DNA of the clones obtained, selected on Luria-Bertani medium (LB, bactotryptone 10 g/l, yeast extract 5 g/l, NaCl 10 g/l, Agar 15 g/l, pH 7.5) supplemented with kanamycin (50 mg/l), was extracted by the alkaline lysis method (Birnboim and Doly, 1979) and verified by enzymatic digestions and by sequencing. The resulting plasmid selected was called pMRT1105 (3508 bp) and is represented in FIG. 1. Its complete sequence SEQ.ID01 is given in the sequence listing.

2. Synthesis of pMRT1106

The plasmid pMRT1106 (4098 bp) differs from pMRT1105 by the addition of the origin of replication ColE1 of Escherichia coli (<<ori ColE1>>).

The fragment, carrying <<ori ColE1>> (590 bp), was isolated from the plasmid pBR322 marketed by New England Biolabs. The plasmid (5 μg) was digested by NdeI, purified with the aid of the <<QIAquick PCR Purification>> kit and recovered in 30 μl of TE buffer (10 mM Tris-HCl, 1 mM Na₂-EDTA, pH 8.0). The plasmid thus linearized was subjected to the action of 20 units of the Klenow fragment (New England Biolabs) in the presence of 12 μl of 500 mM Tris-HCl, pH 7.5-500 mM MgCl₂, 6 μl of 1M dithiothreitol, 6 μl of each of the 10 mM dNTPs in a reaction volume of 120 μl at 37° C. for 30 min. The linearized plasmid so treated was purified with the aid of the <<QIAquick PCR Purification>> kit and recovered in 30 μl of H2O. It was then digested by AlwNI in order to isolate the fragment carrying <<ori ColE1>>, purified with the aid of the <<QIAquick PCR Purification>> kit and subjected to the action of 6 units of T4 DNA polymerase (New England Biolabs) in a reaction medium of 120 μl in the presence of 12 μl of T4 DNA polymerase×10 buffer, 4 μl of 10 mM dNTP and 6 μl of BSA 1 mg/ml. The reaction was carried out at 37° C. for 30 min. The DNA fragment was then isolated by 1.2% agarose gel electrophoresis in TBE buffer, purified with the aid of the <<QIAquick Gel Extraction>> kit and recovered in 50 μl of H₂O. This DNA fragment was then inserted into the plasmid pMRT1105 (2 μg) digested by StuI, purified with the aid of the <<QIAquick PCR Purification>> kit, dephosphorylated by 50 units of calf intestinal alkaline phosphatase (New England Biolabs) in a final reaction medium of 120 μl in the presence of 12 μl of 3×10 buffer (New England Biolabs) at 37° C. for 1 hour, and purified with the aid of the <<QIAquick PCR Purification>> kit. The ligation by PCR reaction was carried out with 10 ng of digested dephosphorylated pMRT1105 plasmid and 100 ng of DNA fragments carrying <<ori ColE1>> in a reaction medium of 10 μl in the presence of 1 μl of T4 DNA ligase×10 buffer (New England Biolabs) and 400 units of T4 DNA ligase enzyme (New England Biolabs). It is made up of 180 cycles consisting of 2 stages, one of 10° C. for 30 sec. and the other of 30° C. for 30 sec. in the <<GeneAmp PCR System 9700>> thermocycler. The bacteria, Escherichia coli DH5, which had previously been made competent, were transformed (Hanahan, 1983). The validity of the clones obtained was verified by a PCR test on bacterial colonies using 10 pmol of each of the 2 oligodeoxynucleotides, 5′

(SEQ.ID23)

5′ AACCTAGGAAAAGACCGAGCGCCTTTGC 3′ and

(SEQ.ID26)

5′ ATGCATCCAAAATTTTGGTAGAATTTACAAGCTATAAGGTTATTGTC

CTGGG 3′.

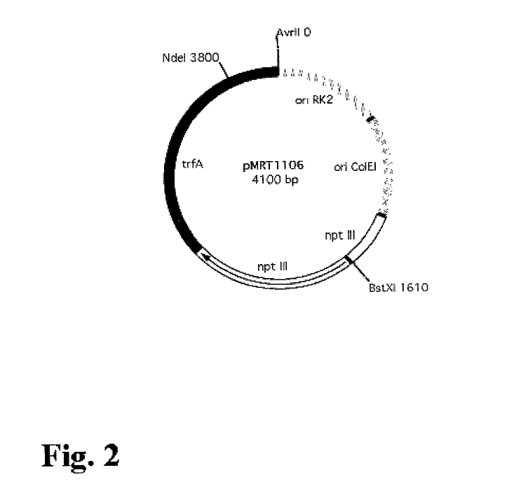

in a reaction medium of 24 μl in the presence of 22 μl of SuperMix PCR (GIBCO BRL Life Technologies). The PCR amplification reaction was carried out in the <<GeneAmp PCR System 9700>> thermocycler. After denaturation at 94° C. for 5 min., the DNA was subjected to 30 cycles each made up of the stages of denaturation at 94° C. for 30 sec., hybridization at 60° C. for 30 sec. and elongation at 72° C. for 30 sec. Then, in the last cycle, the elongation was continued at 72° C. for 7 min. The PCR reaction medium was then subjected to 1% agarose gel electrophoresis in TBE buffer. The insertion of the sequence <<ori ColE1>> was visualized by the presence of a fragment of about 1.6 kbp. The plasmid DNA of the clones obtained, selected on LB medium supplemented with kanamycin (50 mg/l), was extracted by the alkaline lysis method (Birnboim and Doly, 1979) and verified by enzymatic digestions and by sequencing. The resultant plasmid selected was called pMRT1106 (4098 bp) and is represented in FIG. 2. Its complete sequence SEQ.ID02 is given in the sequence listing.

3. Synthesis of pMRT1118

The plasmid pMRT1118 (5971 bp) differs from pMRT1106 by the introduction of a clean transfer DNA (T-DNA) in pMRT1106. This clean T-DNA is made up of the expression cassette of the mutant nptII gene (Frisch et al., 1995) under the control of the promoter and terminator of the nopaline synthase gene (nos, Depicker et al., 1982) of Agrobacterium tumefaciens placed between the right (RB) and left border (LB) of the plasmid pTiT37 of Agrobacterium tumefaciens nopaline strain.

3.1 Synthesis of the Fragment Carrying <<End nptII-Tnos-LB>>

3.1.1. Obtaining the Fragment Carrying LB

The AvrII-SmaI fragment (173 bp) carrying LB (151 bp) was amplified by PCR from 5 ng of pBin19 matrix DNA with the aid of 20 pmoles of each of the 2 oligodeoxynucleotides, 5′ TTCCTAGGTTGACGTCTTCTGATGGGCTGCCTGTATCG 3′ (SEQ.ID29) containing the AvrII and AatII restriction sites, and 5′ CCTATGGATATCCCCCGGGGGATAGCCCCAGTACATTAAAAACGTCC 3′ (SEQ.ID30) possessing the SmaI restriction site, in the presence of 200 μM of each of the dNTPs, 60 mM Tris-SO₄ pH 9.1, 18 mM (NH₄)₂ SO₄, 1.8 mM MgSO₄ and 2 U of ELONGASE enzyme (GIBCO BRL Life Technologies) in a final reaction medium of 50 μl. The PCR amplification reaction was carried out in the <<GeneAmp PCR System 9700>> thermocycler. After denaturation at 94° C. for 3 min., the DNA was subjected to 15 cycles each made up of the stages of denaturation at 94° C. for 30 sec., hybridization at 55° C. for 30 sec. and elongation at 68° C. for 45 sec. Then, in the last cycle, the elongation was continued at 68° C. for 3 min. Forty μl of the PCR reaction medium were then subjected to the action of 12.5 U of the Klenow fragment (New England Biolabs) in the presence of 2 μl of each of the dNTPs at 10 mM. The reaction was carried out at 37° C. for 10 min. The PCR product so treated was then isolated by 2% agarose gel electrophoresis in TBE buffer and purified with the aid of the <<QIAquick Gel Extraction>> kit. The DNA was recovered in 50 μl of H2O.

3.1.2. Obtaining the Fragment Carrying the nos Terminator (Tnos)

The SmaI-BspEI fragment (288 bp) carrying the nos terminator (256 bp) was amplified and treated in the same way as the fragment carrying LB except that the 2 oligodeoxynucleotides used are 5′CTATCCCCCGGGGGATATCCATAGGCCCGATCTAGTAACATAGATGAC 3′ (SEQ.ID31) containing a SmaI restriction site and 5′ GCGCACTTGGGCCCATAGCTCGACGAACGATCGTTCAAACATTTGGC 3′ (SEQ.ID32), possessing the Bsp120I restriction site.

3.1.3. Obtaining the Fragment Carrying <<End of the nptII>> Gene (<<End nptII>>)

The fragment (221 bp) carrying <<end nptII>> (221 bp) was amplified and treated in the same way as the fragment carrying LB except that the 2 oligodeoxynucleotides used are 5′ TTCGTCGAGCTATGGGCCCAAGTGCGCATCCCGTGGGCGAAGAACTC 3′ (SEQ.ID33) containing a Bsp120I restriction site and 5′ TTCTTGACGAGTTCTTCTGAGCGGG 3′ (SEQ.ID34) downstream of BstBI.

3.1.4. Obtaining the Fragment <<End nptII-Tnos-LB>>

The fragments, <<LB>>, <<Tnos>> and <<end nptII>>, were assembled by splicing overlap extension in 2 stages:

assembly of the fragments <<LB>> and Tnos>> resulting in a fragment <<Tnos-LB>>,

assembly of the fragment <<Tnos-LB>> and of the fragment <<end nptII>> resulting in a fragment <<end nptII-Tnos-LB>>.

To do this, a first PCR amplification was carried out from 10 μl of each of the PCR products treated corresponding to <<LB>> and <<Tnos>>, with the aid of 20 pmoles of each of the oligodeoxynucleotides, 5′ TTCCTAGGTTGACGTCTTCTGATGGGCTGCCTGTATCG 3′ (SEQ.ID29) and 5′ GCGCACTTGGGCCCATAGCTCGACGAACGATCGTTCAAACATTTGGC 3′ (SEQ.ID32), in the presence of 200 μM of each of the dNTPs, 60 mM Tris-SO₄ pH 9.1, 18 mM (NH₄)₂ SO₄, 1.8 mM MgSO₄ and 2 U of ELONGASE enzyme (GIBCO BRL Life Technologies) in a final reaction medium of 50 μl. The PCR amplification reaction was carried out in the <<GeneAmp PCR System 9700>> thermocycler. After denaturation at 94° C. for 3 min., the DNA was subjected to 15 cycles each made up of the stages of denaturation at 94° C. for 45 sec., hybridization at 62° C. for 45 sec. and elongation at 68° C. for 1 min. Then, in the last cycle, the elongation was continued at 68° C. for 3 min. Forty μl of the PCR reaction medium were then subjected to the action of 12.5 U of the Klenow fragment (New England Biolabs) in the presence of 2 μl of each of the dNTPs at 10 mM. The reaction was carried out at 37° C. for 10 min. The PCR product so treated, corresponding to the fragment <<Tnos-LB>> (477 bp), was then isolated by 2% agarose gel electrophoresis in TBE buffer and purified with the aid of the <<QIAquick Gel Extraction>> kit. The DNA was recovered in 30 μl of H₂O.

A second PCR amplification was then carried out from 7 μl of each of the PCR products treated, corresponding to <<end nptII>> and <<Tnos-LB>>, with the aid of 20 pmoles of each of the oligodeoxynucleotides, 5′ TTCCTAGGTTGACGTCTTCTGATGGGCTGCCTGTATCG 3′ (SEQ.ID29) and 5′ TTCTTGACGAGTTCTTCTGAGCGGG 3′ (SEQ.ID34), in the presence of 200 μM of each of the dNTPs, 60 mM Tris-SO4 pH 9.1, 18 mM (NH4)2 SO4, 1.8 mM MgSO4 and 2 U of ELONGASE enzyme (GIBCO BRL Life Technologies) in a final reaction medium of 50 μl. The PCR amplification reaction was carried out in the <<GeneAmp PCR System 9700>> thermocycler. After denaturation at 94° C. for 3 min., the DNA was subjected to 15 cycles each made up of the stages of denaturation at 94° C. for 45 sec., hybridization at 60° C. for 45 sec. and elongation at 68° C. for 1 min. Then, in the last cycle, the elongation was continued at 68° C. for 3 min. This reaction is repeated 3 times for the PCR products treated, corresponding to <<end nptII>> and <<Tnos-LB>>. The PCR reaction medium was then subjected to 2% agarose gel electrophoresis in TBE buffer and purified with the aid of the <<QIAquick Gel Extraction>> kit. The DNA fragment (672 bp), corresponding to <<end nptII-Tnos-LB>>, was recovered in 100 μl of H2O. Next, 95 μl of this DNA were hydrolysed by AvrII, purified with the aid of the <<QIAquick PCR Purification>> kit, digested by BstBI, purified with the aid of the <<QIAquick PCR Purification>> kit and recovered in 50 μl of H₂O. The 631 bp AvrII-BstBI fragment carries the sequence <<end nptII-Tnos-LB>>.

3.2. Synthesis of the Fragment Carrying <<End of the nos Promoter (Pnos) and Start of the nptII Gene>> (<<End Pnos-st. nptII>>)

The fragment (1023 bp) carrying <<end Pnos-st. nptII>> was amplified and treated in the same way as the fragment carrying LB except that the 2 oligodeoxynucleotides used are 5′ GGAATCGAAATCTCGTGATGGCAGG 3′ (SEQ.ID39) and 5′ ATTATTGCGCGTTCAAAAGTCGCC 3′ (SEQ.ID40), and that the DNA was subjected to 15 cycles each made up of the stages of denaturation at 94° C. for 45 sec., hybridization at 55° C. for 45 sec. and elongation at 68° C. for 1 min. The amplification was repeated for 2 samples of pBin19 DNA.

3.3. Synthesis of the Fragment <<End Pnos-nptII-Tnos-LB>>

The fragments, <<end Pnos-st. nptII>> and <<end nptII-Tnos-LB>>, were assembled by splicing overlap extension in order to produce a fragment <<end Pnos-nptII-Tnos-LB>>.

To do this, a PCR amplification was carried out from 5 μl of each of the PCR products treated, corresponding to <<end Pnos-st. nptII>> and <<end nptII-Tnos-LB>>, with the aid of 20 pmoles of each of the oligodeoxynucleotides, 5′ TTCCTAGGTTGACGTCTTCTGATGGGCTGCCTGTATCG 3′ (SEQ.ID29) and 5′ ATTATTGCGCGTTCAAAAGTCGCC 3′ (SEQ.ID40), in the presence of 200 μM of each of the dNTPs, 60 mM Tris-SO₄ pH 9.1, 18 mM (NH₄)₂ SO₄, 1.8 mM MgSO₄ and 2 U of ELONGASE enzyme (GIBCO BRL Life Technologies) in a final reaction medium of 50 μl. The PCR amplification reaction was carried out in the <<GeneAmp PCR System 9700>> thermocycler. After denaturation at 94° C. for 3 min., the DNA was subjected to 15 cycles each made up of the stages of denaturation at 94° C. for 45 sec., hybridization at 55° C. for 45 sec. and elongation at 68° C. for 1 min. Then, in the last cycle, the elongation was continued at 68° C. for 3 min. This reaction was repeated 5 times for each of the PCR products treated, corresponding to <<end Pnos-st. nptII>> and <<end nptII-Tnos-LB>>. The PCR reaction medium was then subjected to 0.7% agarose gel electrophoresis in TBE buffer and purified with the aid of the <<QIAquick Gel Extraction>> kit. The DNA fragment (1608 bp) corresponding to <<end Pnos-nptII-Tnos-LB>>, was recovered in 50 μl of H2O. Next, this DNA was subjected to the action of 62.5 U of the Klenow fragment (New England Biolabs) in the presence of 15 μl of the Klenow fragment×10 buffer (New England Biolabs) and 10 μl of each of the dNTPs at 10 mM. The reaction was carried out at 37° C. for 10 min. The DNA so treated was then purified with the aid of the <<QIAquick PCR Purification>> kit and recovered in 50 μl of H₂O.

3.4. Synthesis of the Fragment Carrying <<RB-MCS-st. Pnos>>

3.4.1. Obtaining the Fragment Carrying <<RB and Multiple Cloning Site (MCS)>> (<<RB-MCS>>)

The fragment (210 bp) carrying <<RB-MCS>> was amplified and treated in the same way as the fragment carrying LB except that the 2 oligodeoxynucleotides used are 5′ CGGTACCGAAGCTTTGAATTCACTCGAGCAGATTGTCGTTTCCCGCC 3′ (SEQ.ID35) possessing the restriction sites KpnI, HindIII, EcoRI and XhoI, and 5′ TATCCTAGGAACCGGTAAACCCTGTGGTTGGCATGC 3′ (SEQ.ID36) possessing the restriction sites AvrII and AgeI.

3.4.2. Obtaining the Fragment Carrying <<Multiple Cloning Site (MCS) and Start of the nos Promoter (Pnos)>> (<<MCS-st. Pnos>>)

The fragment (209 bp) carrying <<MCS-st. Pnos>> was amplified and treated in the same way as the fragment carrying LB except that the 2 oligodeoxynucleotides used are 5′ ATATGAGACTCTAATTGGATACCGAGGGG 3′ (SEQ.ID37) and 5′ GCTCGAGTGAATTCAAAGCTTCGGTACCGTTGAAGGAGCCACTCAGCCG 3′ (SEQ.ID38) possessing the restriction sites XhoI, EcoRI, HindIII and KpnI.

3.4.3. Obtaining the Fragment Carrying <<RB-MCS-st. Pnos>>

The fragments, <<RB-MCS>> and <<MCS-st. Pnos>>, were assembled by splicing overlap extension in order to produce a fragment <<RB-MCS-st. Pnos>>.

To do this, a PCR amplification was carried out from 12 μl of each of the PCR products treated, corresponding to <<RB-MCS>> and <<MCS-st. Pnos>>, with the aid of 20 pmoles of each of the oligodeoxynucleotides, 5′ ATATGAGACTCTAATTGGATACCGAGGGG 3′ (SEQ.ID37) and 5′ TATCCTAGGAACCGGTAAACCCTGTGGTTGGCATGC 3′ (SEQ.ID36), in the presence of 200 μM of each of the dNTPs, 60 mM Tris-SO4 pH9.1, 18 mM (NH4)2 SO4, 1.8 mM MgSO4 and 2 U of ELONGASE enzyme (GIBCO BRL Life Technologies) in a final reaction medium of 50 μl. The PCR amplification reaction was carried out in the <<GeneAmp PCR System 9700>> thermocycler. After denaturation at 94° C. for 3 min., the DNA was subjected to 15 cycles each made up of the stages of denaturation at 94° C. for 30 sec., hybridization at 60° C. for 30 sec. and elongation at 68° C. for 45 sec. Then, in the last cycle, the elongation was continued at 68° C. for 3 min. This reaction was repeated for the remaining 3×12 μl of each of the PCR products treated, corresponding to <<RB-MCS>> and <<MCS-st. Pnos>>. The PCR reaction medium was then subjected to 2% agarose gel electrophoresis in TBE buffer and purified with the aid of the <<QIAquick Gel Extraction>> kit (QIAGEN). The DNA fragment (390 bp) corresponding to <<RB-MCS-st. Pnos>>, was recovered in 100 μl of H2O. Next, 95 μl of this DNA was hydrolysed by AvrII, purified with the aid of the <<QIAquick PCR Purification>> kit, digested by Bsu36I, purified with the aid of the <<QIAquick PCR Purification>> kit and recovered in 50 μl of H₂O. The 295 bp AvrII-Bsu36I fragment carries the sequence <<RB-MCS-st. Pnos>>.

3.5. Synthesis of the T-DNA

The fragments, <<RB-MCS-st. Pnos>> and <<end Pnos-nptII-Tnos-LB>>, were assembled by splicing overlap extension in order to produce a fragment <<RB-MCS-Pnos-nptII-Tnos-LB>> which corresponds to the T-DNA (1883 bp).

To do this, a PCR amplification was carried out from 4 μl of the PCR product treated corresponding to <<RB-MCS-st. Pnos>> and from 5 μl of the PCR product treated corresponding to <<end Pnos-nptII-Tnos-LB>>, with the aid of 20 pmoles of each of the oligodeoxynucleotides, 5′ TTCCTAGGTTGACGTCTTCTGATGGGCTGCCTGTATCG 3′ (SEQ.ID29) and 5′ TATCCTAGGAACCGGTAAACCCTGTGGTTGGCATGC 3′ (SEQ.ID36), in the presence of 200 μM of each of the dNTPs, 60 mM Tris-SO4 pH 9.1, 18 mM (NH4)2 SO4, 1.8 mM MgSO4 and 2 U of ELONGASE enzyme (GIBCO BRL Life Technologies) in a final reaction medium of 50 μl. The PCR amplification reaction was carried out in the <<GeneAmp PCR System 9700>> thermocycler. After denaturation at 94° C. for 3 min., the DNA was subjected to 15 cycles each made up of the stages of denaturation at 94° C. for 45 sec., hybridization at 55° C. for 45 sec. and elongation at 68° C. for 1 min. Then, in the last cycle, the elongation was continued at 68° C. for 3 min. This reaction was repeated 10 times for each of the PCR products treated, corresponding to <<RB-MCS-st. Pnos>> and <<end Pnos-nptII-Tnos-LB>>. The PCR reaction medium was then subjected to 0.8% agarose gel electrophoresis in TBE buffer and purified with the aid of the <<QIAquick Gel Extraction>> kit. The DNA fragment (1883 bp) corresponding to the T-DNA was recovered in 100 μl of H2O. Next, this T-DNA was hydrolysed by AvrII (1873 bp fragment), purified with the aid of the <<QIAquick PCR Purification>> kit and recovered in 100 μl of H₂O.

3.6. Obtaining pMRT1118

The binary plasmid pMRT1118 (5971 bp) results from the introduction of the T-DNA fragment digested by AvrII into the AvrII site of the dephosphorylated pMRT1106 plasmid.

To do this, the pMRT1106 plasmid DNA (5 μg) was digested by AvrII, purified with the aid of the <<QIAquick PCR Purification>> kit, then dephosphorylated by 50 units of calf intestinal alkaline phosphatase (New England Biolabs) in a final reaction medium of 120 μl in the presence of 12 μl of 3×10 buffer (New England Biolabs) at 37° C. for 1 hour, isolated by 0.6% agarose gel electrophoresis in TBE buffer, purified with the aid of the <<QIAquick Gel Extraction>> kit, dephosphorylated a second time with the calf intestinal alkaline phosphatase enzyme under the aforesaid conditions, and finally purified with the aid of the <<QIAquick PCR Purification>> kit and recovered in 50 μl of H₂O.

The ligation by PCR reaction was carried out with 32.5 ng of digested dephosphorylated pMRT1106 plasmid and 50 ng of digested T-DNA fragments in a reaction medium of 10 μl in the presence of 1 μl of T4 DNA ligase×10 buffer (New England Biolabs) and 400 units of T4 DNA ligase enzyme (New England Biolabs). It is made up of 180 cycles consisting of 2 stages, one of 10° C. for 30 sec. and the other of 30° C. for 30 sec., in the <<GeneAmp PCR System 9700>> thermocycler.

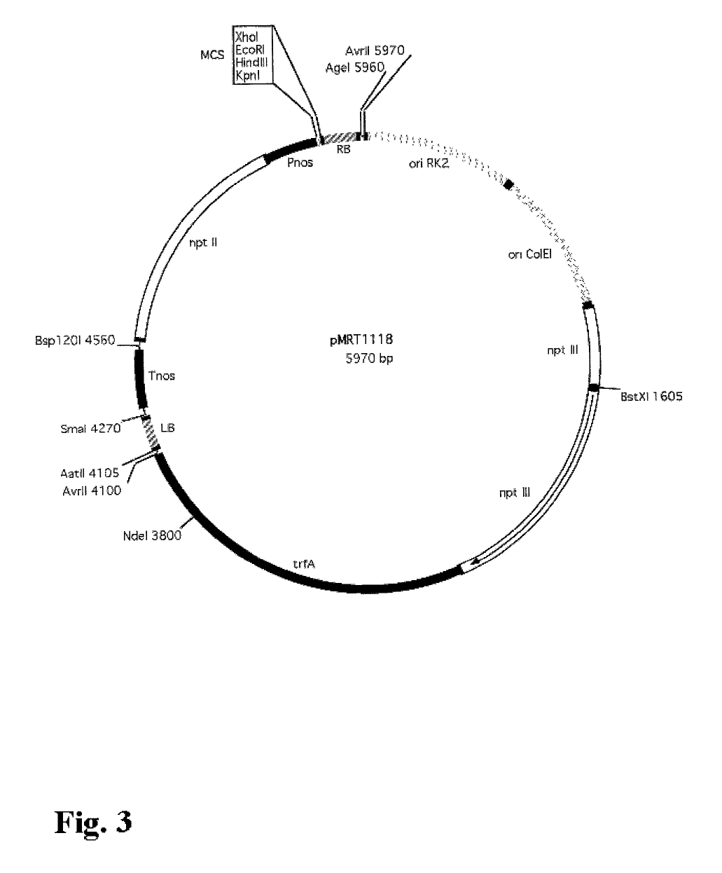

The bacteria, Escherichia coli DH5 which had previously been made competent, were transformed (Hanahan, 1983). The plasmid DNA of the clones obtained, selected on LB medium supplemented with kanamycin (50 mg/l), was extracted by the alkaline lysis method (Birnboim and Doly, 1979) and verified by enzymatic digestions and by sequencing. The resultant plasmid selected was called pMRT1118 (FIG. 3). Its complete sequence SEQ.ID03 is given in the sequence listing.

4. Synthesis of pMRT1119

The plasmid pMRT1119 (6016 bp) differs from pMRT1118 by the addition of additional unique restriction sites in the multiple cloning site MCS of pMRT1118.

4.1. Obtaining the Additional Unique Restriction Sites

The additional unique restriction sites (XbaI, SalI, PacI, BamHI, MluI, HpaI and FseI) were created by hybridization between the 2 oligodeoxynucleotides, 5′AGCTTGGCCGGCCGTTAACACGCGTGGATCCTTAATTAAGTCGACTCTAGAG 3′ (SEQ.ID41) and 5′ AATTCTCTAGAGTCGACTTAATTAAGGATCCACGCGTGTTAACGGCCGGCCA 3′ (SEQ.ID42). To do this, 5 μg of each of the 2 oligodeoxynucleotides were mixed and held at 85° C. for 1 min., followed by a progressive reduction of the temperature to 80° C. for 5 min., then a slow reduction of the temperature to 60° C. in a water bath, and finally a rapid reduction of the temperature to the ambient temperature outside the water bath.

4.2. Obtaining pMRT1119

The binary plasmid pMRT1119 (6016 bp) results from the introduction of the sequence carrying the unique restriction sites into the HindIII and EcoRI sites of the pMRT1118 plasmid.

To do this, the pMRT1118 plasmid DNA (5 μg) was doubly digested by HindIII and EcoRI, purified with the aid of the <<QIAquick PCR Purification>> kit, and recovered in 50 μl of H2O.

The ligation by PCR reaction was carried out with 75 ng of digested pMRT1118 plasmid and 500 ng of fragments carrying the unique restriction sites (described in 4.1.) in a reaction medium of 10 μl in the presence of 1 μl of T4 DNA ligase×10 buffer (New England Biolabs) and 400 units of T4 DNA ligase enzyme (New England Biolabs). It is made up of 180 cycles consisting of 2 stages, one of 10° C. for 30 sec. and the other of 30° C. for 30 sec. in the <<GeneAmp PCR System 9700>> thermocycler.

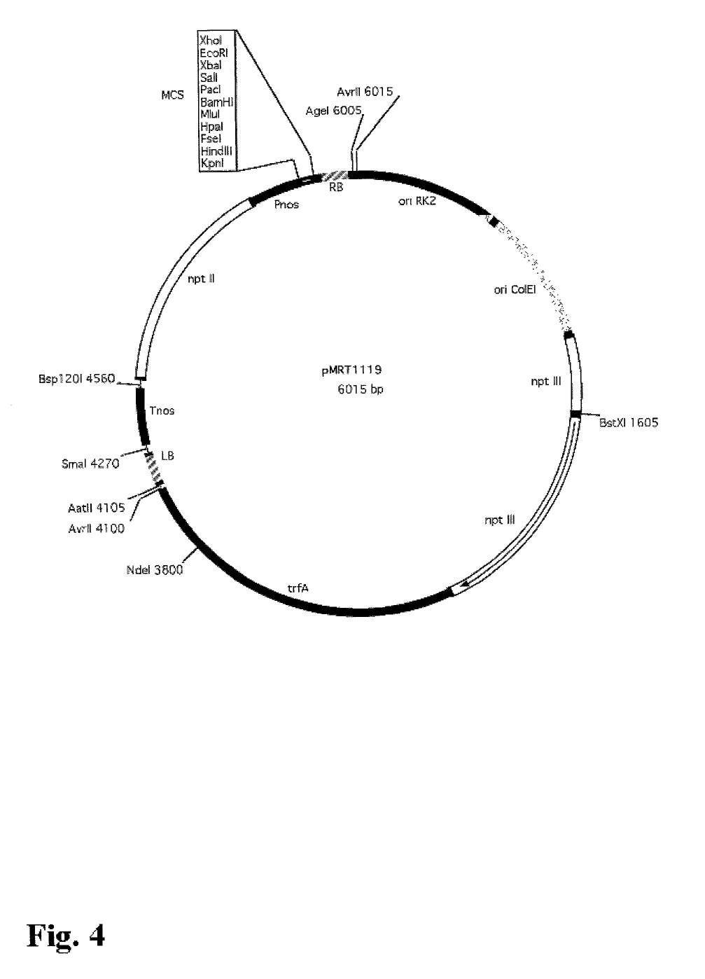

The bacteria, Escherichia coli DH5, which had previously been made competent, were transformed (Hanahan, 1983). The plasmid DNA of the clones obtained, selected on LB medium supplemented with kanamycin (50 mg/l), was extracted by the alkaline lysis method (Birnboim and Doly, 1979) and verified by enzymatic digestions and by sequencing. The resultant plasmid selected was called pMRT1119 (FIG. 4). Its complete sequence SEQ.ID04 is given in the sequence listing.

5. Synthesis of pMRT1121

The plasmid pMRT1121 (6017 bp) results from the insertion into pMRT1119 of a unique restriction site, BspEI, between the nopaline synthase promoter of Agrobacterium tumefaciens (Pnos) and the mutant nptII gene.

The BspEI site was inserted by the assembly by splicing overlap extension of two fragments obtained by PCR amplification.

5.1. Synthesis of the Fragment Carrying “Part of nptII and BspEI Site”

The 892 bp fragment carrying “part of nptII and BspEI site” was amplified by PCR from 5 ng of pBin19 matrix DNA with the aid of 20 pmoles of each of the 2 oligodeoxynucleotides, 5′ GGAATCGAAATCTCGTGATGGCAGG 3′ (SEQ.ID39) and 5′ TAATCTGCATCCGGATCTGGATCGTTTCGC 3′ (SEQ.ID43) carrying the BspEI site, in the presence of 200 μM of each of the dNTPs, 60 mM Tris-SO4 pH 9.1, 18 mM (NH4)2 SO4, 1.8 mM MgSO4 and 2 U of eLONGase enzyme (GIBCO BRL Life Technologies) in a final reaction medium of 50 μl. This reaction was repeated. The PCR amplification reaction was carried out in a “GeneAmp PCR System 9700” thermocycler. After denaturation at 94° C. for 3 min., the DNA was subjected to 15 cycles each made up of the stages of denaturation at 94° C. for 45 sec., hybridization at 55° C. for 45 sec. and elongation at 68° C. for 1 min. Then, in the last cycle, the elongation was continued at 68° C. for 3 min. The PCR product so obtained was isolated by 2% agarose gel electrophoresis in TBE buffer and purified with the aid of the <<QIAquick Gel Extraction>> kit. The DNA was recovered in 50 μl of H₂O.

5.2. Synthesis of the Fragment Carrying “BspEI Site-MCS”

The fragment (250 bp) carrying “BspEI site-MCS” was amplified by PCR from 5 ng of pMRT1118 matrix DNA with the aid of 20 pmoles of each of the 2 oligodeoxynucleotides, 5′ GCTCGAGTGAATTCAAAGCTTCGGTACCGTTGAAGGAGCCACTCAGCCG 3′ (SEQ.ID38) and 5′ ACGATCCAGATCCGGATGCAGATTATTTGG 3′ (SEQ.ID44) carrying the BspEI site. The conditions of PCR amplification and treatment of the PCR fragment obtained are the same as described in 5.1.

5.3. Synthesis of the Fragment Carrying “Part nptII-MCS”

The 1117 bp fragment carrying “part nptII-MCS” results from the assembly of the 2 PCR fragments, “part nptII-BspEI site” and “BspEI site-MCS”, by splicing overlap extension.

To do this, a PCR amplification was carried out from 7.5 μl of each of the PCR products treated, corresponding to “part nptII-BspEI site” and “BspEI site-MCS”, with the aid of 20 pmoles of each of the 2 oligodeoxynucleotides, 5′ GGAATCGAAATCTCGTGATGGCAGG 3′ (SEQ.ID39) and 5′ GCTCGAGTGAATTCAAAGCTTCGGTACCGTTGAAGGAGCCACTCAGCCG 3′ (SEQ.ID38), in the presence of 200 μM of each of the dNTPs, 60 mM Tris-SO₄ pH 9.1, 18 mM (NH4)2 SO4, 1.8 mM MgSO₄ and 2 U of eLONGase enzyme (GIBCO BRL Life Technologies) in a final reaction medium of 50 μl. This reaction was repeated 4 times. The PCR amplification reaction was carried out in a “GeneAmp PCR System 9700” thermocycler. After denaturation at 94° C. for 3 min., the DNA was subjected to 15 cycles each made up of the stages of denaturation at 94° C. for 45 sec., hybridization at 62° C. for 45 sec. and elongation at 68° C. for 1 min. Then, in the last cycle, the elongation was continued at 68° C. for 7 min. The PCR product so obtained was isolated by 1% agarose gel electrophoresis in TBE buffer and purified with the aid of the “QIAquick Gel Extraction” kit. The DNA was recovered in 50 μl of H2O, digested by Bsu36I and PstI, and subjected to 2% agarose gel electrophoresis in TEB buffer. The 315 bp DNA fragment was isolated and purified with the aid of the “QIAquick Gel Extraction” kit.

5.4. Obtaining pMRT1121

The 315 bp DNA fragment Bsu36I-PstI, which carries the BspEI site, was ligated to the Bsu36I and PstI sites of the binary plasmid pMRT1119.

The plasmid pMRT1119 was previously digested by Bsu36I and PstI, subjected to 1% agarose gel electrophoresis in TEB buffer and purified with the aid of the <<QIAquick Gel Extraction>> kit. The DNA was recovered in 50 μl of H₂O, dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, and purified with the aid of the “QIAquick PCR Purification” kit.

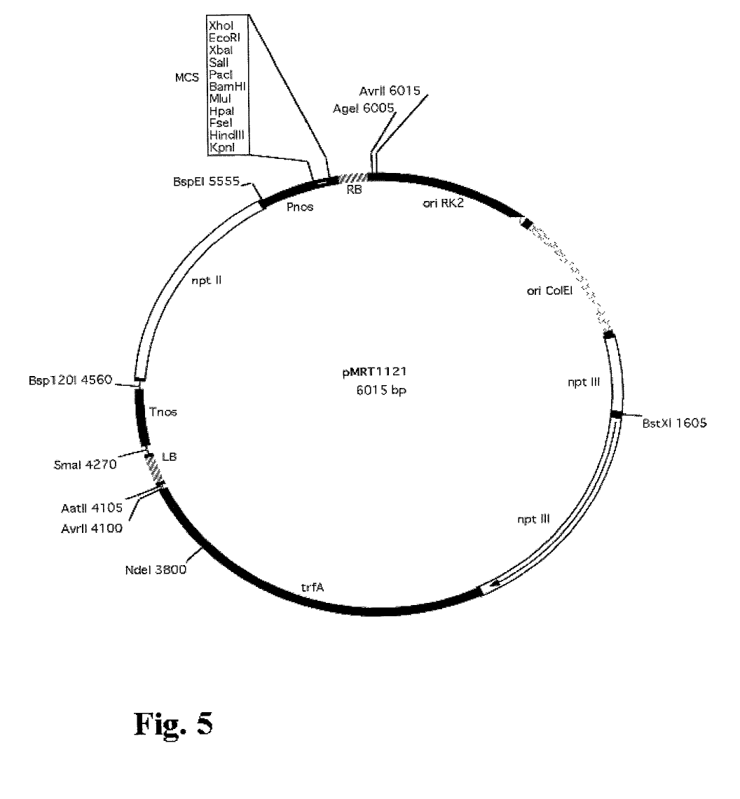

The ligation by PCR reaction was carried out with 100 ng of digested dephosphorylated pMRT1119 plasmid and 30 ng of digested DNA fragments (315 bp) in a reaction medium of 10 μl in the presence of 1 μl of T4 DNA ligase×10 buffer (New England Biolabs) and 400 units of T4 DNA ligase enzyme (New England Biolabs). It is made up of 180 cycles consisting of 2 stages, one of 10° C. for 30 sec. and the other of 30° C. for 30 sec., in a “GeneAmp PCR System 9700” thermocycler. The bacteria, Escherichia coli DH5 or SCS110 (deficient in Dam and Dcm methylases), which had previously been made competent, were transformed (Hanahan, 1983). The plasmid DNA of the clones obtained, selected on LB medium supplemented with kanamycin (50 mg/l), was extracted by the alkaline lysis method and verified by enzymatic digestions and sequencing. The resultant plasmid was called pMRT1121 (FIG. 5). Its complete sequence SEQ.ID05 is given in the sequence listing.

6. Synthesis of pMRT1122

The plasmid pMRT1122 (6016 bp) results from the introduction into pMRT1119 of a point mutation in the mutant nptII gene in order to restore the BglII restriction site and thus lead to the obtaining of the wild-type nptII gene.

To do this, the BglII restriction site was restored in the mutant nptII gene of pMRT1119 by directed mutagenesis in accordance with the Michael procedure (1994).

The oligodeoxynucleotide 5′ ATGGGTCACGACGAGATCTTCGCCGTCGGG 3′ (SEQ.ID45) was previously phosphorylated by subjecting 600 pmoles of the oligodeoxynucleotide to the action of 90 units of T4 kinase (Amersham) in the presence of 30 μl of T4 kinase×10 buffer (Amersham) and 3 μl of 100 mM ATP in a final reaction medium of 300 μl at 37° C. for 30 min. The oligodeoxynucleotide so treated was purified with the aid of the <<Qiaquick Removal Nucleotide>> (QIAGEN) kit in accordance with the supplier's recommendations.

The ligation by PCR was then carried out from 10 ng of pBIN19 matrix with the aid of 200 pmoles of each of the oligodeoxynucleotides, 5′ GGAATCGAAATCTCGTGATGGCAGG 3′ (SEQ.ID39), 5′ ATGGGTCACGACGAGATCTTCGCCGTCGGG 3′ (SEQ.ID45), which was phosphorylated, including the BglII restriction site, and 5′ ATTATTGCGCGTTCAAAAGTCGCC 3′ (SEQ.ID40), in the presence of 400 μM of each of the dNTPs, 10 μl of Taq DNA ligase×10 buffer (New England BioLabs), 5 units of Vent DNA Polymerase (New England BioLabs) and 40 units of Taq DNA ligase (New England BioLabs) in a final reaction medium of 100 μl. The PCR ligation reaction was carried out in the <<GeneAmp PCR System 9700>> thermocycler by carrying out the following three successive phases: the first phase consisted of a cycle made up of the stages of denaturation at 94° C. for 5 min., hybridization at 50° C. for 1 min. and elongation at 65° C. for 4 min.; the second phase was made up of 28 cycles each comprising the stages of denaturation at 94° C. for 30 sec., hybridization at 50° C. for 1 min. and elongation at 65° C. for 4 min.; and, finally, the last phase consisted of a cycle made up of the stages of denaturation at 94° C. for 30 sec., hybridization at 50° C. for 1 min. and elongation at 65° C. for 15 min. The PCR reaction medium was then subjected to 0.8% agarose gel electrophoresis in TBE buffer and purified with the aid of the <<QIAquick Gel Extraction>> kit. The DNA fragments (1023 bp) so treated were hydrolysed by NcoI and PstI and subjected to 2% agarose gel electrophoresis. The 383 bp DNA fragments were isolated, purified with the aid of the <<QIAquick Gel Extraction>> kit, recovered in 50 μl of H2O and ligated to the NcoI and PstI sites of the pMRT1119 plasmid. To do this, the pMRT1119 plasmid DNA was digested by NcoI and PstI, purified on 0.8% agarose gel. The fragment corresponding to the plasmid was isolated, purified with the aid of the <<QIAquick Gel Extraction>> kit, then dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, and, finally, recovered in 50 μl of H₂O.

The ligation by PCR was carried out with 50 ng of digested dephosphorylated pMRT1119 plasmid and all the DNA fragments digested and treated in a reaction medium of 10 μl in the presence of 1 μl of T4 DNA ligase×10 buffer (New England Biolabs) and 400 units of T4 DNA ligase enzyme (New England Biolabs). It is made up of 180 cycles consisting of 2 stages, one of 10° C. for 30 sec. and the other of 30° C. for 30 sec., in the <<GeneAmp PCR System 9700>> thermocycler.

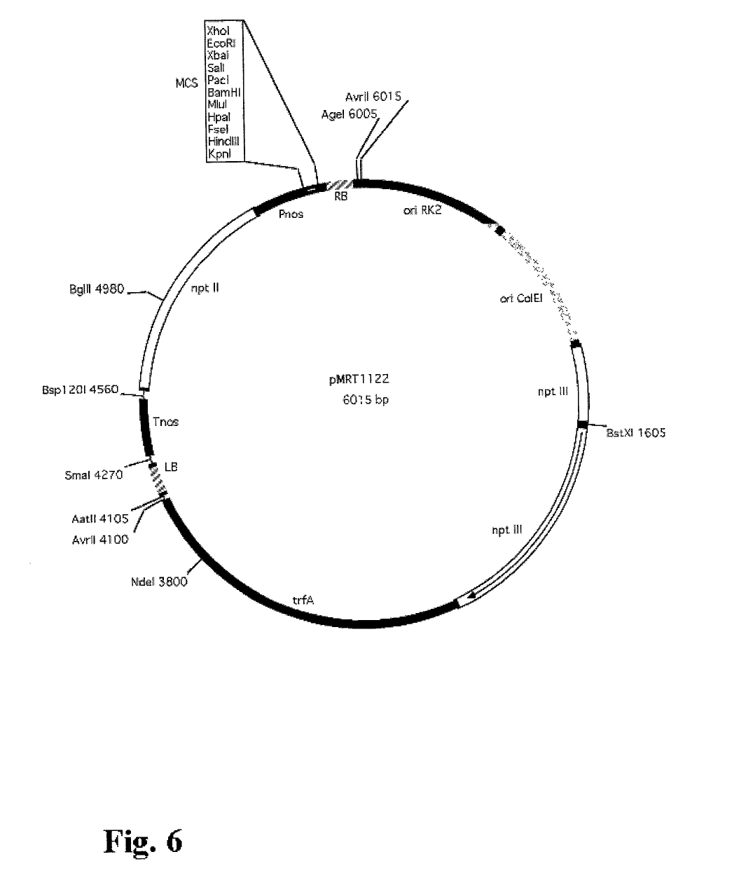

The bacteria, Escherichia coli DH5, which had previously been made competent, were transformed (Hanahan, 1983). The plasmid DNA of the clones obtained, selected on LB medium supplemented with kanamycin (50 mg/l), was extracted by the alkaline lysis method (Birnboim and Doly, 1979) and screened by digestion by BglII. The plasmid DNA of the clone selected was verified by enzymatic digestions and by sequencing. The resultant plasmid selected was called pMRT1122 (6016 bp). It is represented in FIG. 6 and its complete sequence SEQ.ID06 is given in the sequence listing.

7. Synthesis of pMRT1155

The plasmid pMRT1155 (6017 bp) differs from pMRT1122 by the presence of the BspEI site.

To do this, the 315 bp insert fragment containing the BspEI site was obtained by digestion of pMRT1121 by Bsu36I and PstI, 1.2% agarose gel electrophoresis in TEB buffer, purification with the aid of the “QIAquick Gel Extraction” kit and recovery in 50 μl of H₂O.

The pMRT1122 vector fragment was obtained by digestion of pMRT1122 by Bsu36I and PstI, 1.2% agarose gel electrophoresis in TEB buffer, purification with the aid of the “QIAquick Gel Extraction” kit and recovery in 50 μl of H₂O. After that, digested pMRT1122 was dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, and purified with the aid of the “QIAquick PCR Purification” kit.

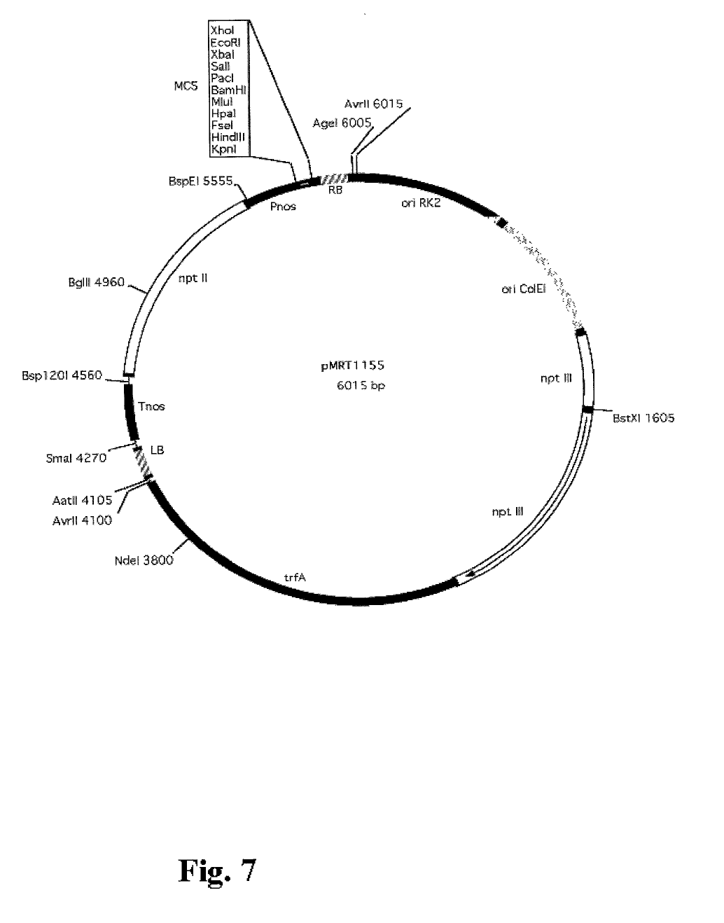

The ligation by PCR reaction was carried out with 100 ng of dephosphorylated digested pMRT1122 plasmid and 50 ng of digested DNA fragments (315 bp) in a reaction medium of 10 μl in the presence of 1 μl of T4 DNA ligase×10 buffer (New England Biolabs) and 400 units of T4 DNA ligase enzyme (New England Biolabs). It is made up of 180 cycles consisting of 2 stages, one of 10° C. for 30 sec. and the other of 30° C. for 30 sec. in a “GeneAmp PCR System 9700” thermocycler. The bacteria, Escherichia coli DH5 or SCS110 (deficient in Dam and Dcm methylases), which had previously been made competent, were transformed (Hanahan, 1983). The plasmid DNA of the clones obtained, selected on LB medium supplemented with kanamycin (50 mg/l), was extracted by the alkaline lysis method and verified by enzymatic digestions and sequencing. The resultant plasmid was called pMRT1155 (FIG. 7). Its complete sequence SEQ.ID07 is given in the sequence listing.

8. Obtaining Basic Binary Plasmids Comprising the Sequence “eP35S-T35S”

8.1. Obtaining pMRT1205

The plasmid pMRT1205 (7503 bp) includes the expression cassette of the mutant nptII gene and a “double 35S promoter-35S terminator” sequence (eP35S-T35S) for the cloning of genes of interest. It results from the cloning of the eP35S promoter of the cauliflower mosaic virus (CaMV) into pMRT1175. The CaMV eP35S promoter corresponds to a duplication of the transcription-activating sequences situated upstream of the TATA element of the 35S promoter (Kay et al., 1987). As for the plasmid pMRT1175 (6767 bp), this results from the cloning of T35S of the cauliflower mosaic virus into pMRT1121. The transcription-terminating sequence, CaMV T35S, corresponds to the non-coding region in 3′ of the circular double-stranded DNA sequence of the cauliflower mosaic virus producing the 35S transcript (Franck et al., 1980).

8.1.1. Obtaining pMRT1175

The plasmid pMRT1175 results from the cloning of the EcoRI-XhoI insert DNA fragments corresponding to the T35S into the EcoRI and XhoI sites of the vector pMRT1121 produced from Escherichia coli strain SCS110.

The pMRT1121 vector fragment was obtained by digestion of 7 μg of pMRT1121 by EcoRI and XhoI, purification with the aid of the “QIAquick PCR Purification” kit and recovery in 50 μl of H2O. After that, digested pMRT1121 was dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, and purified with the aid of the “QIAquick PCR Purification” kit and recovered in 50 μl of H2O.

The EcoRI-XhoI insert DNA fragments (757 bp) corresponding to the T35S (750 bp) were obtained by digestion by EcoRI and XhoI of the plasmid pJIT163, which derives from the plasmid pJIT60 (Guerineau and Mullineaux, 1993). They were then subjected to 1% agarose gel electrophoresis in TEB buffer, purified with the aid of the <<QIAquick Gel Extraction)>> kit and recovered in 30 μl of H2O.

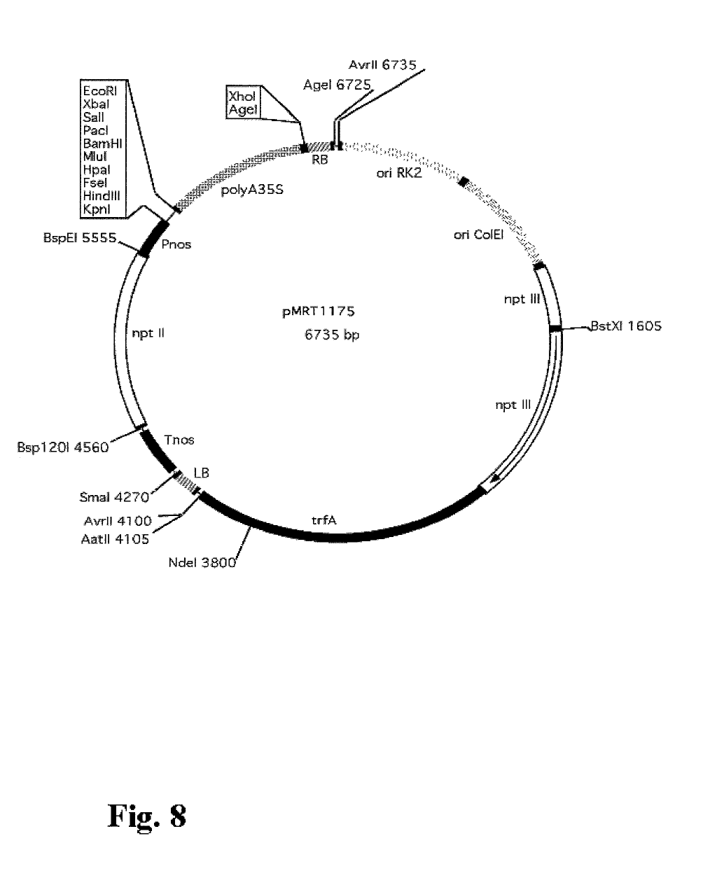

The ligation by PCR reaction was carried out as described earlier in 7, with 80 ng of dephosphorylated digested pMRT1121 plasmid and 80 ng of digested insert DNA fragments. The bacteria, Escherichia coli DH5, which had previously been made competent, were transformed. The plasmid DNA of the clones obtained, selected on LB medium supplemented with kanamycin (50 mg/l), was extracted by the alkaline lysis method and verified by enzymatic digestions and sequencing. The resultant plasmid was called pMRT1175 (6767 bp). It is represented in FIG. 8 and its complete sequence SEQ.ID08 is given in the sequence listing.

8.1.2. Obtaining pMRT1205

The plasmid pMRT1205 results from the cloning of the KpnI-HindIlI insert DNA fragments corresponding to eP35S into the KpnI and HindIII sites of the vector pMRT1175.

The pMRT1175 vector fragment was obtained by digestion of 4.1 μg of pMRT1175 by KpnI and HindIII, purification with the aid of the <<Concert Rapid PCR Purification System>> kit and recovery in 100 μl of H₂O. After that, digested pMRT1175 was dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, purified with the aid of the <<Concert Rapid PCR Purification System>> kit, and recovered in 50 μl of H₂O.

The KpnI-HindIII insert DNA fragments (743 bp) corresponding to eP35S (735 bp) were obtained by digestion by KpnI and HindIII of the plasmid pJIT163, which derives from the plasmid pJIT60 (Guerineau and Mullineaux, 1993). They were then subjected to 1% agarose gel electrophoresis in TEB buffer, purified with the aid of the “QIAquick Gel Extraction” kit and recovered in 30 μl of H₂O.

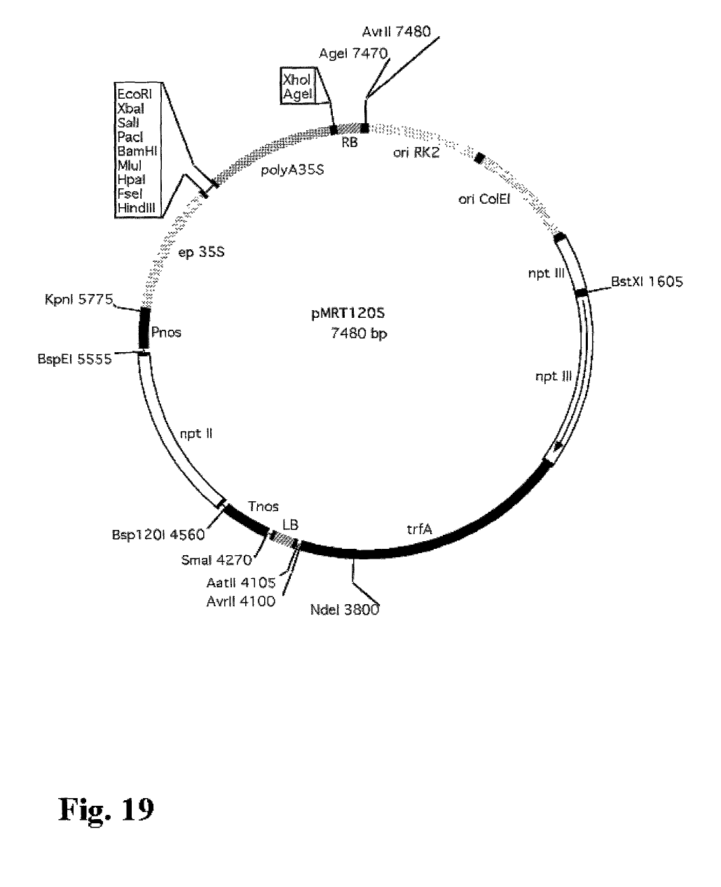

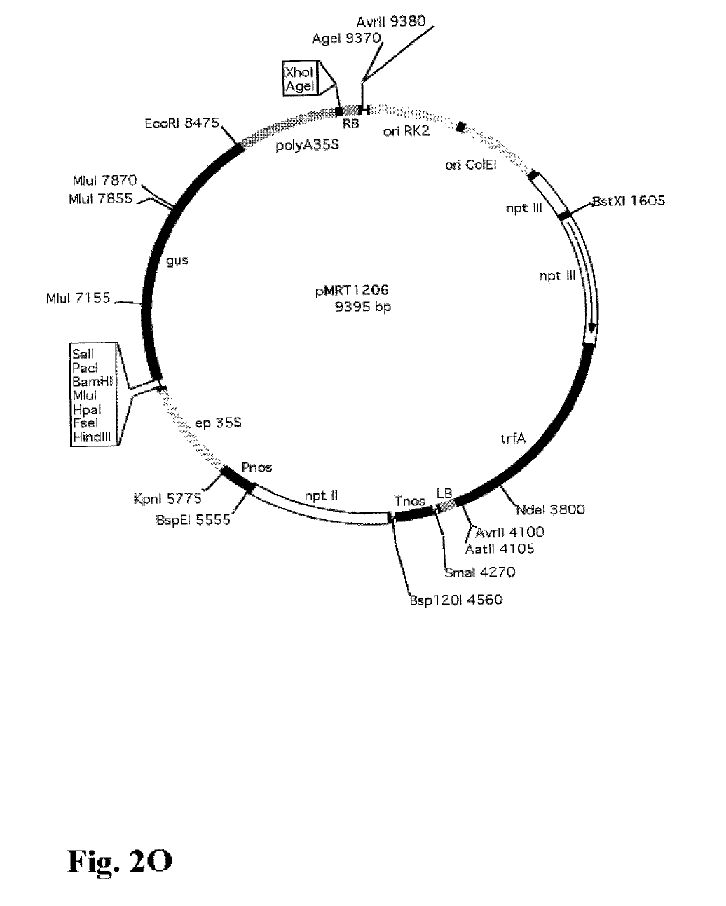

The ligation by PCR reaction was carried out as described earlier in 7, with 48 ng of dephosphorylated digested pMRT1175 plasmid and 30 ng of digested insert DNA fragments. The bacteria, Escherichia coli DH5, which had previously been made competent, were transformed. The plasmid DNA of the clones obtained, selected on LB medium supplemented with kanamycin (50 mg/l), was extracted by the alkaline lysis method and verified by enzymatic digestions and sequencing. The resultant plasmid was called pMRT1205 (7503 bp). It is represented in FIG. 19 and its complete sequence SEQ.ID19 is given in the sequence listing.

8.2. Obtaining pMRT1203

The plasmid pMRT1203 (7503 bp) includes the expression cassette of the wild-type nptII gene and a “double 35S promoter-35S terminator” sequence (eP35S-T35S) for the cloning of genes of interest. It results from the cloning of the eP35S promoter of the cauliflower mosaic virus into pMRT1176. As for the plasmid pMRT1176 (6767 bp), this results from the cloning of T35S of the cauliflower mosaic virus into pMRT1155. The eP35S promoter and the T35S terminator are described in 8.1.

8.2.1. Obtaining pMRT1176

The plasmid pMRT1176 results from the cloning of the EcoRI-XhoI insert DNA fragments (757 bp) corresponding to the T35S (750 bp) into the EcoRI and XhoI sites of the vector pMRT1155 produced from Escherichia coli strain SCS110.

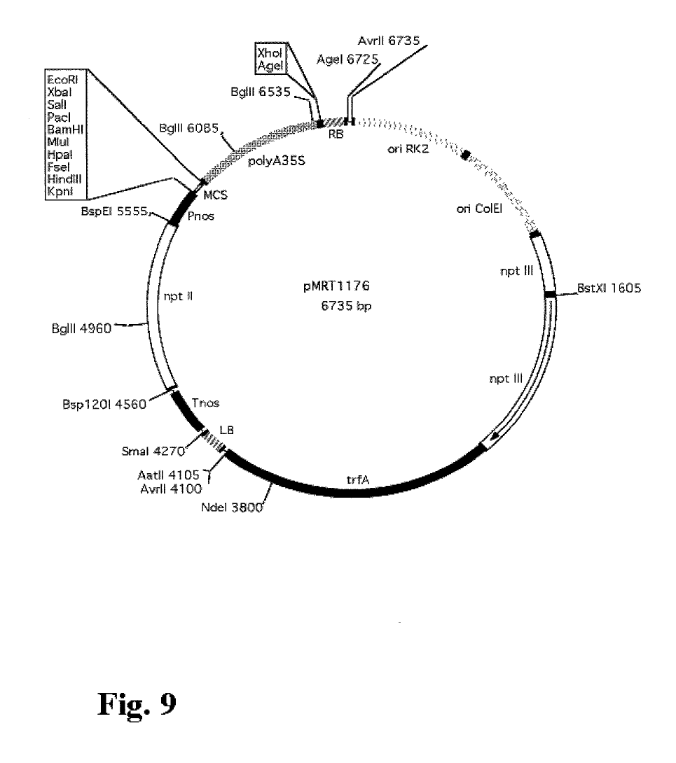

The plasmid pMRT1176 was obtained in accordance with the methodologies described in 8.1.1., except that 6.3 μg of the vector pMRT1155, which constitutes the cloning vector, were digested and treated. The plasmid pMRT1176 (6767 bp) is represented in FIG. 9. Its complete sequence SEQ.ID09 is given in the sequence listing.

8.2.2. Obtaining pMRT1203

The plasmid pMRT1203 results from the cloning of the KpnI-HindIII insert DNA fragments (743 bp) corresponding to eP35S (735 bp) into the KpnI and HindIII sites of the vector pMRT1176.

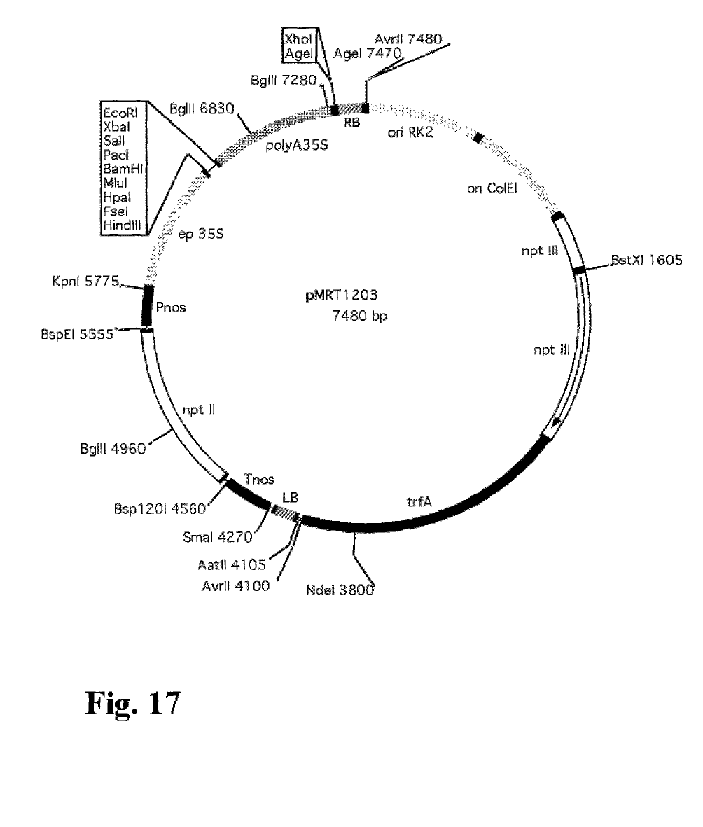

The plasmid pMRT1203 was obtained in accordance with the methodologies described in 8.1.2., except that 3.9 μg of the vector pMRT1176, which constitutes the cloning vector, were digested and treated. The plasmid pMRT1203 (7503 bp) is represented in FIG. 17. Its complete sequence SEQ.ID17 is given in the sequence listing.

9. Obtaining Binary Plasmids Comprising the Sequence “eP35S-uidA-T35S”

These vectors were employed in transformation of tobacco in order to evaluate them and determine the incidence of the mutation of the nptII gene.

9.1. Obtaining pMRT1206

The plasmid pMRT1206 (9390 bp) includes the expression cassette of the mutant nptII gene and the expression cassette of the uidA gene (gus). It results from the cloning of the eP35S promoter of the cauliflower mosaic virus into pMRT1196. As for the plasmid pMRT1196 (8654 bp), this results from the cloning of the uidA gene (Jefferson R A et al., 1986) into pMRT1175.

9.1.1. Obtaining pMRT1196

The plasmid pMRT1196 results from the cloning of the insert DNA fragments (SmaI-“SacI+T4 DNA polymerase”), corresponding to the uidA gene, into the “XbaI+Klenow” site of the vector pMRT1175.

The pMRT1175 vector fragment was obtained by digestion of 10 μg of pMRT1175 by XbaI, purified with the aid of the “QIAquick PCR Purification” kit, recovered in 50 μl of H2O and subjected to the action of 20 units of the Klenow fragment (New England Biolabs) in the presence of 12 μl of 500 mM Tris-HCl pH 7.5, 500 mM MgCl₂, 6 μl of 1M dithiothreitol, 6 μl of each of the 10 mM dNTPs in a reaction volume of 120 μl at 37° C. for 30 min. It was then purified with the aid of the “QIAquick PCR Purification” kit and recovered in 50 μl of H₂O. The plasmid pMRT1175 so digested and treated was dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, and purified with the aid of the “QIAquick PCR Purification” kit and recovered in 50 μl of H₂O.

The insert DNA fragments (2 μg) corresponding to the uidA gene (1.8 kbp) were obtained by digestion of pBI221 (marketed by Clontech) by SacI, purification with the aid of the “QIAquick PCR Purification” kit and recovery in 50 μl of H2O. After that, digested pBI221 was subjected to the action of 6 units of T4 DNA polymerase (New England Biolabs) in a reaction medium of 120 μl in the presence of 12 μl of T4 DNA polymerase×10 buffer, 4 μl of 10 mM dNTPs and 6 μl of BSA 1 mg/ml. The reaction was carried out at 37° C. for 30 min. The plasmid pBI221 so treated was purified with the aid of the “QIAquick PCR Purification” kit and recovered in 50 μl of H2O. Finally, pBI221 so treated was digested by SmaI. The <<[SacI+T4 DNA polymerase]−SmaI>> fragment (1882 bp) was isolated by 0.8% agarose gel electrophoresis, purified with the aid of the “QIAquick Gel Extraction” kit and recovered in 50 μl of H₂O.

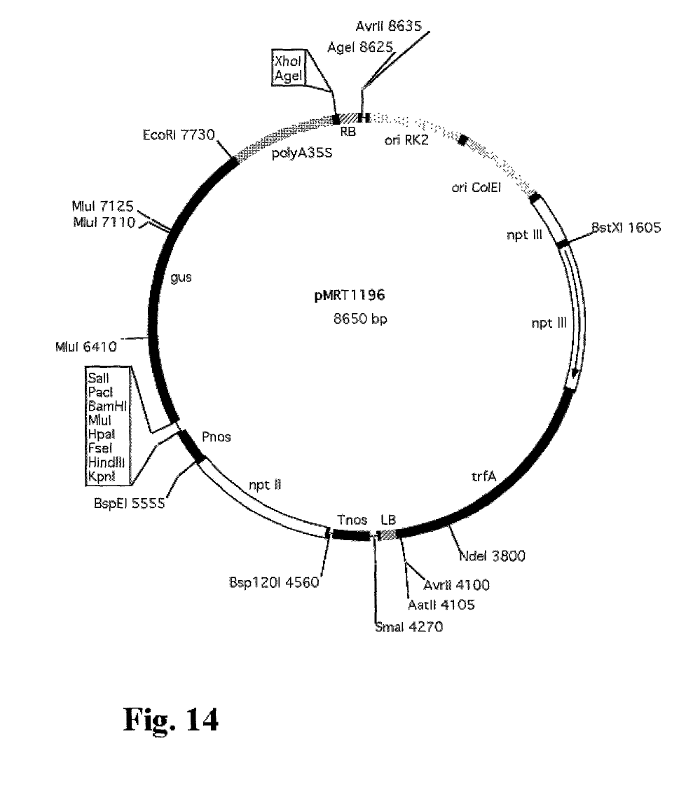

The ligation by PCR reaction was carried out in accordance with the methodologies described in 7, with 15 ng of dephosphorylated digested pMRT1175 plasmid and 100 ng of digested insert DNA fragments. The bacteria, Escherichia coli DH5, which had previously been made competent, were transformed. The plasmid DNA of the clones obtained, selected on LB medium supplemented with kanamycin (50 mg/l), was extracted by the alkaline lysis method and verified by enzymatic digestions and sequencing. The resultant plasmid was called pMRT1196 (8654 bp). It is represented in FIG. 14 and its complete sequence SEQ.ID14 is given in the sequence listing.

9.1.2. Obtaining pMRT1206

The plasmid pMRT1206 (9390 bp) results from the cloning of the KpnI-HindIlI insert DNA fragments (743 bp), corresponding to eP35S (735 bp), into the KpnI and HindIII sites of the vector pMRT1196.

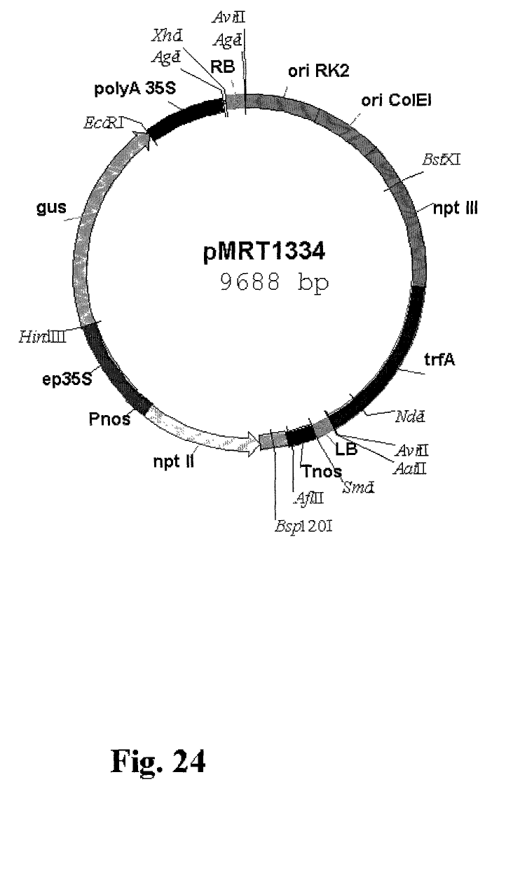

The plasmid pMRT1206 was obtained in accordance with the methodologies described in 8.1.2., except that 2 μg of the vector pMRT1196, which constitutes the cloning vector, were digested and treated. It is represented in FIG. 24 and its complete sequence SEQ.ID24 is given in the sequence listing.

The plasmid pMRT1206 was introduced into Agrobacterium tumefaciens strain LBA4404 by direct transformation in accordance with the procedure of Holsters et al. (1978).

9.2. Obtaining pMRT1204

The plasmid pMRT1204 (9390 bp) includes the expression cassette of the wild-type nptII gene and the expression cassette of the uidA gene. It results from the cloning of the eP35S promoter of the cauliflower mosaic virus into pMRT1192. As for the plasmid pMRT1192 (8654 bp), this results from the cloning of the uidA gene into pMRT1176.

9.2.1. Obtaining pMRT1192

The plasmid pMRT1192 results from the cloning of the insert DNA fragments (SmaI-“SacI+T4 DNA polymerase”), corresponding to the uidA gene, into the “XbaI+Klenow” site of the vector pMRT1176.

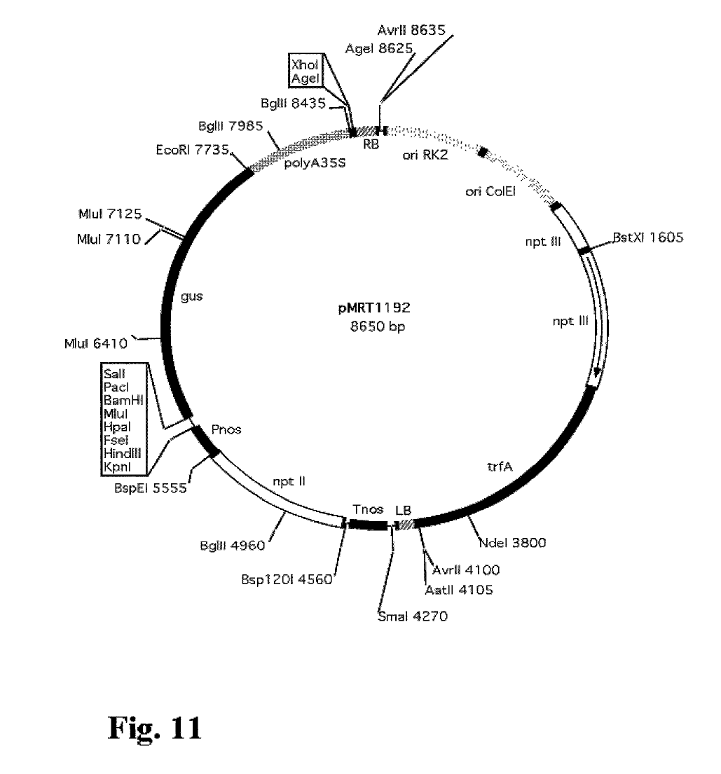

The plasmid pMRT1192 (8654 bp) was obtained in accordance with the methodologies described in 9.1.2., except that the cloning vector is pMRT1176. It is represented in FIG. 11 and its sequence SEQ.ID11 is given in the sequence listing.

9.2.2. Obtaining pMRT1204

The plasmid pMRT1204 (9390 bp) results from the cloning of the KpnI-HindIII insert DNA fragments (743 bp), corresponding to eP35S (735 bp), into the KpnI and HindIII sites of the vector pMRT1192.

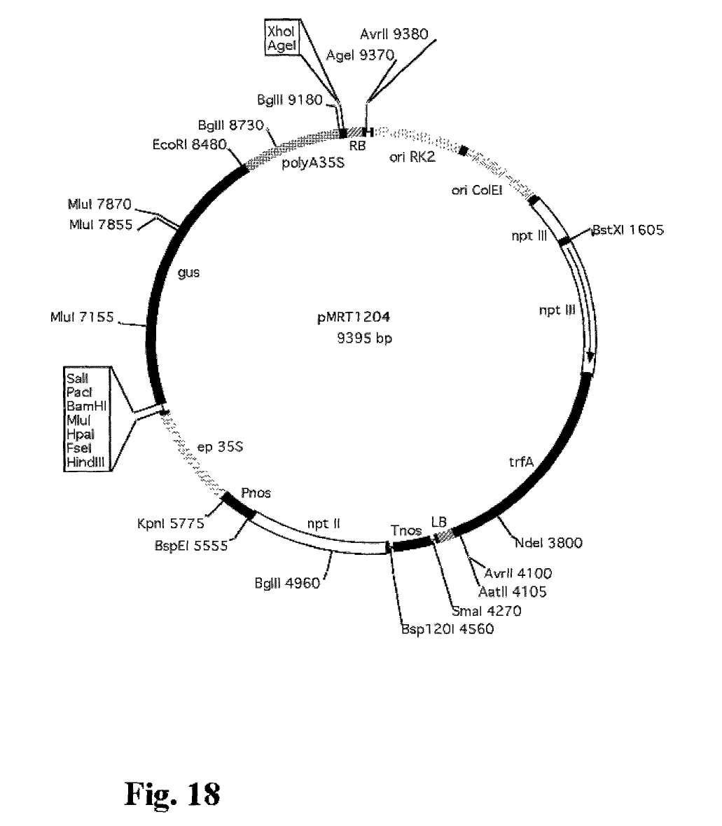

The plasmid pMRT1204 was obtained in accordance with the methodologies described in 8.1.2., except that 2 μg of the vector pMRT1192, which constitutes the cloning vector, were digested and treated. It is represented in FIG. 18 and its complete sequence SEQ.ID18 is given in the sequence listing.

The plasmid pMRT1204 was introduced into Agrobacterium tumefaciens strain LBA4404 by direct transformation in accordance with the procedure of Holsters et al. (1978).

10. Obtaining Binary Plasmids Containing the Bar Gene

The final plasmids were employed in transformation of maize and/or tobacco in order to evaluate them.

10.1. Creation of pMRT1210

The plasmid pMRT1210 (10003 bp) includes the expression cassette of the bar gene and the expression cassette of the uidA gene. It results from the cloning of the expression cassette of the uidA gene into pMRT1195. As for the plasmid pMRT1195 (6865 bp), this results from the cloning of the sequence “promoter followed by intron 1 of the rice actin gene” (McElroy D et al., 1991) into pMRT1191. The plasmid pMRT1191 (4805 bp) derives from pMRT1119.

10.1.1. Obtaining pMRT1191

The plasmid pMRT1191 (4805 bp) differs from pMRT1119 by the deletion of the nos promoter and mutant nptII gene sequences.

It was obtained by digestion by Bsp120I of 10 μg of pMRT1119, followed by purification with the aid of the “QIAquick PCR Purification” kit and recovery in 50 μl of H2O. After that, digested pMRT1119 and [sic] was hydrolysed by KpnI, purified with the aid of the “QIAquick PCR Purification” kit, recovered in 50 μl of H2O, subjected to the action of 6 units of T4 DNA polymerase (New England Biolabs) in a reaction medium of 120 μl in the presence of 12 μl of T4 DNA polymerase×10 buffer, 4 μl of 10 mM dNTPs and 6 μl of BSA at 1 mg/ml. The reaction was carried out at 37° C. for 30 min. The vector fragment was then isolated by 0.8% agarose gel electrophoresis, purified with the aid of the “QIAquick Gel Extraction” kit and recovered in 50 μl of H₂O.

Thirty nanograms of the plasmid so treated were religated by ligation by PCR reaction (procedure described in 7).

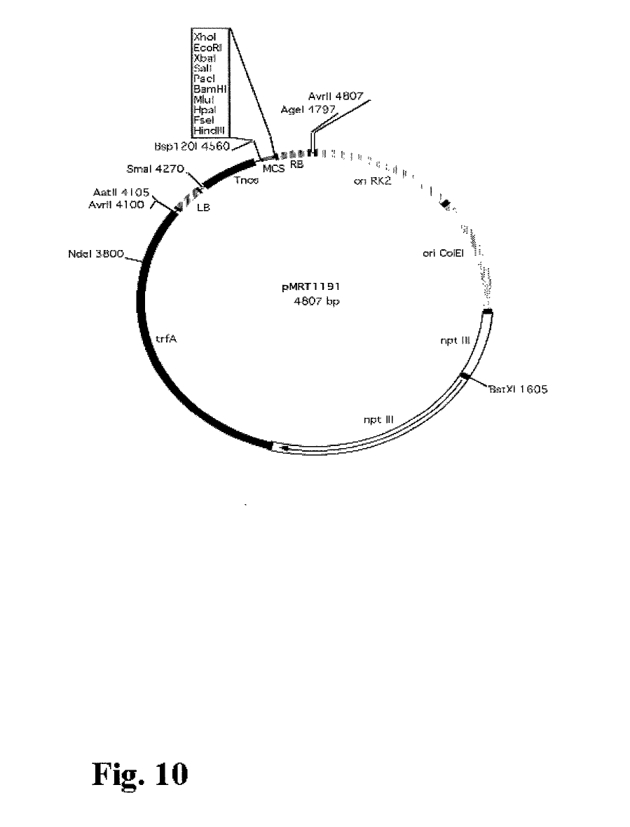

The resultant plasmid was called pMRT1191. It is represented in FIG. 10 and its complete sequence SEQ.ID10 is given in the sequence listing.

10.1.2. Obtaining pMRT1201

The plasmid pMRT1201 (7943 bp) differs from pMRT1191 by the insertion of the expression cassette of the uidA gene isolated from the plasmid pUC19-Phmwg-IA-uidA-Tnos which comprises the uidA expression cassette inserted into the EcoRI and HindIII sites of pUC19 (marketed by New England Biolabs). This plasmid results from successive clonings. The fragment corresponding to the sequence <<uidA gene-nos terminator>> was isolated by successive digestions by <<EcoRI followed by the action of the Klenow fragment>> and SmaI from pBI221 SacI (=pBI221 (Clontech) whose ScaI site was eliminated by SacI digestion followed by the action of T4 DNA polymerase, then ligation). The isolated and purified fragment was inserted into the <<SmaI−[SphI+T4 DNA polymerase]>> sites of pUC19 (Clontech) in order to obtain the resultant plasmid pUC19-uidA-Tnos. After that, the EcoRI site recreated on the 3′ side of Tnos was eliminated by replacement of the BstBI-HindIII fragment of pUC19-uida-Tnos with the BstBI-HindIII fragment obtained by digestion of the fragment amplified by PCR (1054 bp) from the pUC19-uidA-Tnos matrix with the aid of the 2 oligodeoxynucleotides 5′ AGGCATTGGTTTCGAAGCG 3′ (SEQ.ID46) containing the BstBI site and 5′ TACGCCAAGCTTGGCAATTCC 3′(SEQ.ID47). The resultant plasmid was called pUC19-uidA-Tnos EcoRI. Then, in order to introduce a NcoI site at the level of the initiator codon of the uidA gene, a 440 bp fragment was amplified by PCR from the pUC19-uidA-Tnos EcoRI matrix with the aid of the 2 oligodeoxynucleotides 5′ AATACCCGGGACCATGGTCCGTCCTGTAG 3′ (SEQ.ID48) containing the NcoI and SmaI sites and 5′ ATAGTCTGCCAGTTCAGTTCGTTG 3′ (SEQ.ID49) situated downstream of SnaBI. The PCR fragment digested by SmaI and SnaBI was then inserted into pUC19-uidA-Tnos

EcoRI in order to replace the existing SmaI-SnaBI fragment. The resultant plasmid was called pUC19-NcoI-uidA-Tnos. After that, the Phmwg promoter [High Molecular Weight Glutenin, Anderson et al. (1989)], carried by the EcoRI-SmaI fragment, was introduced into the EcoRI and SmaI sites of pUC19-NcoI-uidA-Tnos in order to produce the plasmid pUC19-Phmwg-uidA-Tnos. Finally, the intron 1 of the rice actin gene (IA, EcoRV-SmaI fragment) comes from the plasmid pAct1-F6 (McElroy D et al., 1991) which is itself modified by the addition of restriction sites notably on both sides of the EcoRV-SmaI fragment containing IA. The SmaI-NcoI fragment carrying IA was isolated from pACt1-F6 modified and inserted into the SmaI and NcoI sites of pUC19-Phmwg-uidA-Tnos in order to produce the plasmid pUC19-Phmwg-IA-uidA-Tnos. The techniques used are the ones already described in this patent.

The pMRT1191 vector fragment was obtained by digestion of 2 μg of pMRT1191 by EcoRI, purification with the aid of the <<Concert Rapid PCR Purification System>> kit, followed by the action of 20 units of the Klenow fragment (New England Biolabs) in the presence of 12 μl of 500 mM Tris-HCl pH 7.5, 500 mM MgCl₂, 6 μl of 1M dithiothreitol, 6 μl of each of the dNTPs at 10 mM in a reaction volume of 120 μl at 37° C. for 30 min., purification with the aid of the <<Concert Rapid PCR Purification System>> kit, and recovery in 50 μl of H2O. After that, digested and treated pMRT1191 was dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, purified with the aid of the <<Concert Rapid PCR Purification System>> kit, and recovered in 50 μl of H₂O.

The insert DNA fragments (2 μg) corresponding to the expression cassette of the uidA gene (3 kbp), viz. the uidA gene placed under the control of the wheat “high molecular weight glutenin” promoter followed by the intron 1 of the rice actin gene and the nopaline synthase terminator of Agrobacterium tumefaciens, were obtained by digestion of pUC19-Phmwg-IA-uidA-Tnos by ScaI (site present in pUC19), purification with the aid of the “QIAquick PCR Purification” kit, recovery in 50 μl of H₂O, digestion by HindIII, purification with the aid of the “QIAquick PCR Purification” kit, recovery in 50 μl of H₂O, treatment by 20 units of the Klenow fragment (New England Biolabs) in the presence of 12 μl of 500 mM Tris-HCl, pH 7.5, 500 mM MgCl₂, 6 μl of 1M dithiothreitol, 6 μl of each of the 10 mM dNTPs in a reaction volume of 120 μl at 37° C. for 30 min., purification with the aid of the “QIAquick PCR Purification” kit, recovery in 50 μl of H₂O and digestion by EcoRI. After that, the insert DNA fragments were isolated by 0.8% agarose gel electrophoresis, purified with the aid of the “QIAquick Gel Extraction” kit, subjected to the action of the Klenow fragment as described above, and recovered in 50 μl of H₂O.

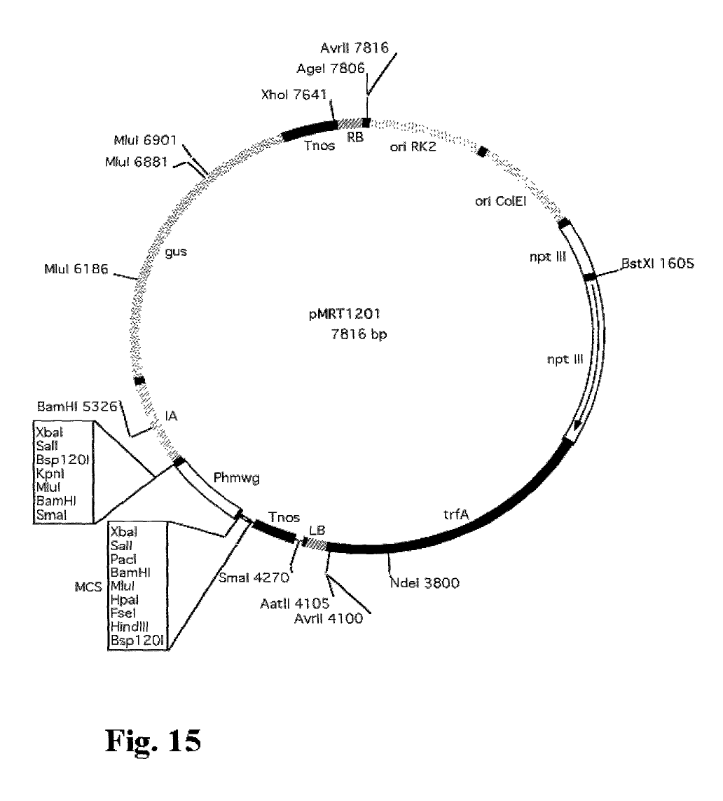

The ligation by PCR reaction was carried out as described earlier in 7, with 10 ng of dephosphorylated digested pMRT1191 plasmid and 100 ng of digested and treated insert DNA fragments. The bacteria, Escherichia coli DH5, which had previously been made competent, were transformed. The plasmid DNA of the clones obtained, selected on LB medium supplemented with kanamycin (50 mg/l), was extracted by the alkaline lysis method and verified by enzymatic digestions and sequencing. The resultant plasmid was called pMRT1201 (7943 bp). It is represented in FIG. 15 and its complete sequence SEQ.ID15 is given in the sequence listing.

10.1.3. Obtaining pMRT1210

The plasmid pMRT1210 (10003 bp) differs from pMRT1201 by the insertion of the sequence “promoter followed by the intron 1 of the rice actin gene—bar gene” (Pact-IA-bar) isolated from pSB12-Pact-IA-bar-Tnos which derives from pSB12 described by Komari et al. (1996). The plasmid pSB12-Pact-IA-bar-Tnos results from the cloning of the expression cassette <<Pact-IA-bar-Tnos>> (BspDI−<<XhoI+Klenow>> fragment), isolated from pDM302, into the SmaI and BspDI sites of pSB12, whose XhoI site on the LB side was deleted. The plasmid pDM302, constructed in the laboratory of Wu R., comprises the expression cassette <<Pact-IA-bar-Tnos>> in the plasmid pSP72 marketed by Promega.

The pMRT1201 vector fragment was obtained by digestion of 2 μg of pMRT1201 by HpaI, purification with the aid of the <<Concert Rapid PCR Purification System>> kit, and recovery in 98 μl of H2O. After that, digested pMRT1201 was dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, purified with the aid of the <<Concert Rapid PCR Purification System>> kit, and recovered in 50 μl of H2O.

The insert DNA fragments (2 μg) corresponding to “Pact-IA-bar” (2.1 kbp) were obtained by XbaI digestion of pBIOS273, purification with the aid of the “QIAquick PCR Purification” kit, recovery in 50 μl of H₂O, treatment with 20 units of the Klenow fragment (New England Biolabs) in the presence of 12 μl of 500 mM Tris-HCl, pH 7.5-500 mM MgCl₂, 6 μl of 1M dithiothreitol and 6 μl of each of the 10 mM dNTPs in a reaction volume of 120 μl at 37° C. for 30 min. The insert DNA fragments so treated were then isolated by 0.8% agarose gel electrophoresis, purified with the aid of the “QIAquick PCR Purification” kit and recovered in 50 μl of H₂O.

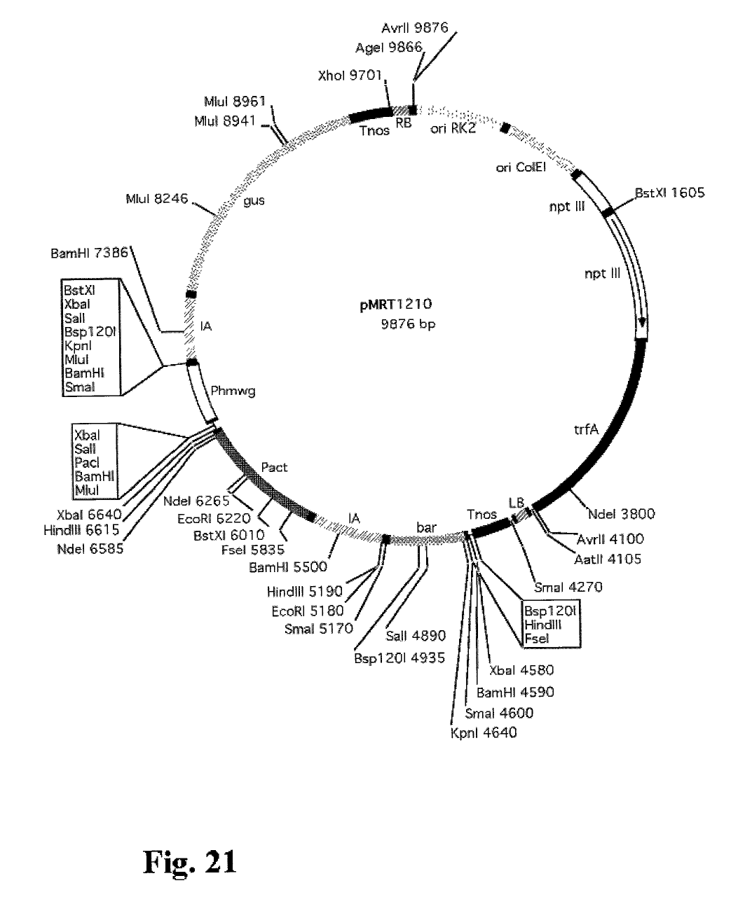

The ligation by PCR reaction was carried out as described earlier in 7, with 60 ng of dephosphorylated digested pMRT1201 plasmid and 100 ng of digested and treated insert DNA fragments. The bacteria, Escherichia coli DH5, which had previously been made competent, were transformed. The plasmid DNA of the clones obtained, selected on LB medium supplemented with kanamycin (50 mg/l), was extracted by the alkaline lysis method and verified by enzymatic digestions and sequencing. The resultant plasmid was called pMRT1210. It is represented in FIG. 21 and its complete sequence SEQ.ID21 is given in the sequence listing.

This plasmid pMRT1210 was then introduced into Agrobacterium tumefaciens strain LBA4404 (pSB1) [Komari T et al., 1996] and strain LBA4404 by direct transformation in accordance with the procedure of Holsters et al. (1978).

10.1.4. Obtaining pMRT1193

The binary plasmid pMRT1193 was obtained by cloning, into the EcoRI and [XhoI+Klenow fragment action] sites of pMRT1119, of the <<EcoRI−[HindIII+Klenow fragment action]>> fragment carrying the sequence <<Phmwg-IA-uidA-Tnos>> isolated from pUC19-Phmwg-IA-uidA-Tnos, described in 10.1.2.

To do this, the pMRT1119 vector fragment was obtained by digestion of 10 μg of pMRT1119 by XhoI, purification with the aid of the “QIAquick PCR Purification” kit, recovery in 50 μl of H2O, treatment with 20 units of the Klenow fragment (New England Biolabs) in the presence of 12 μl of 500 mM Tris-HCl, pH 7.5-500 mM MgCl₂, 6 μl of 1M dithiothreitol and 6 μl of each of the 10 mM dNTPs in a reaction volume of 120 μl at 37° C. for 30 min. Then purification with the aid of the “QIAquick PCR Purification” kit and digestion by EcoRI. The treated vector fragment was isolated by 0.8% agarose gel electrophoresis, purified with the aid of the “QIAquick PCR Purification” kit and recovered in 50 μl of H₂O. After that, digested pMRT1119 was dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, purified with the aid of the “QIAquick PCR Purification” kit and recovered in 50 μl of H₂O.

The insert DNA fragments (2 μg) corresponding to the expression cassette of the uidA gene (3 kbp), viz. the uidA gene placed under the control of the wheat “high molecular weight glutenin” promoter followed by the intron 1 of the rice actin gene and the nopaline synthase terminator of Agrobacterium tumefaciens, were obtained by digestion of pUC19-Phmwg-IA-uidA-Tnos by ScaI (site present in pUC19), purification with the aid of the “QIAquick PCR Purification” kit, recovery in 50 μl of H₂O, digestion by HindIII, purification with the aid of the “QIAquick PCR Purification” kit, recovery in 50 μl of H₂O, treatment with 20 units of the Klenow fragment (New England Biolabs) in the presence of 12 μl of 500 mM Tris-HCl, pH 7.5, 500 mM MgCl₂, 6 μl of 1M dithiothreitol and 6 μl of each of the 10 mM dNTPs in a reaction volume of 120 μl at 37° C. for 30 min., purification with the aid of the “QIAquick PCR Purification” kit, recovery in 50 μl of H₂O, and digestion by EcoRI. The insert DNA fragments were then isolated by 0.8% agarose gel electrophoresis, purified with the aid of the “QIAquick Gel Extraction” kit and recovered in 50 μl of H₂O.

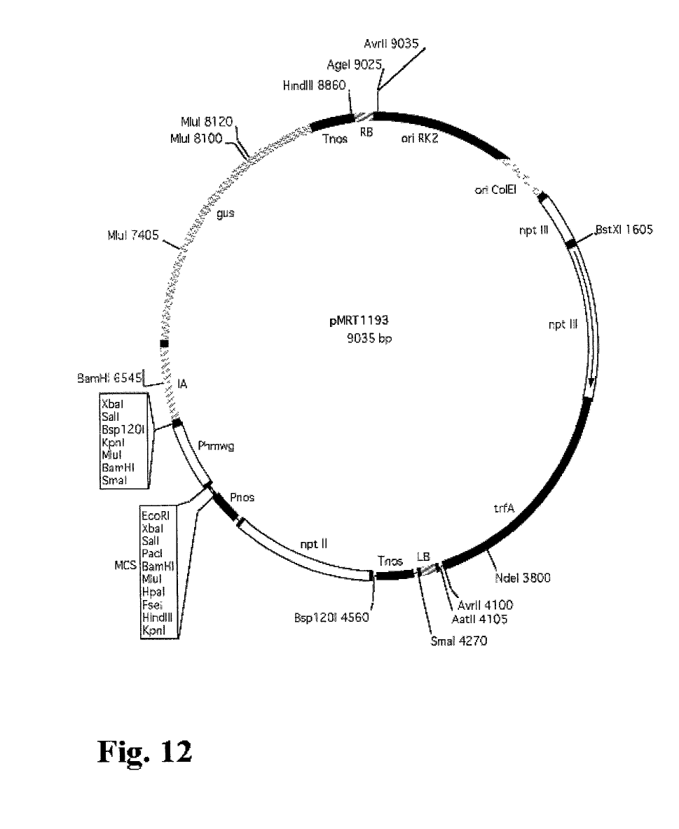

The ligation by PCR reaction was carried out as described earlier in 7, with 20 ng of dephosphorylated digested pMRT1119 plasmid and 100 ng of digested and treated insert DNA fragments. The bacteria, Escherichia coli DH5, which had previously been made competent, were transformed. The plasmid DNA of the clones obtained, selected on LB medium supplemented with kanamycin (50 mg/l), was extracted by the alkaline lysis method and verified by enzymatic digestions and sequencing. The resultant plasmid was called pMRT1193 (9143 bp). It is represented in FIG. 12 and its complete sequence SEQ.ID12 is given in the sequence listing.

10.2. Obtaining pMRT1195

The plasmid pMRT1195 (6857 bp) differs from pMRT1191 by the insertion of the sequence “promoter followed by the intron 1 of the rice actin gene—bar gene” (Pact-IA-bar). The vector pMRT1191 was digested by Bsp120I and KpnI followed by the action of the enzyme T4 DNA polymerase and dephosphorylated but not religated was obtained as described in 10.1.1.

The insert DNA fragments corresponding to “Pact-IA-bar” (2.1 kbp) were obtained as described in 10.1.3.

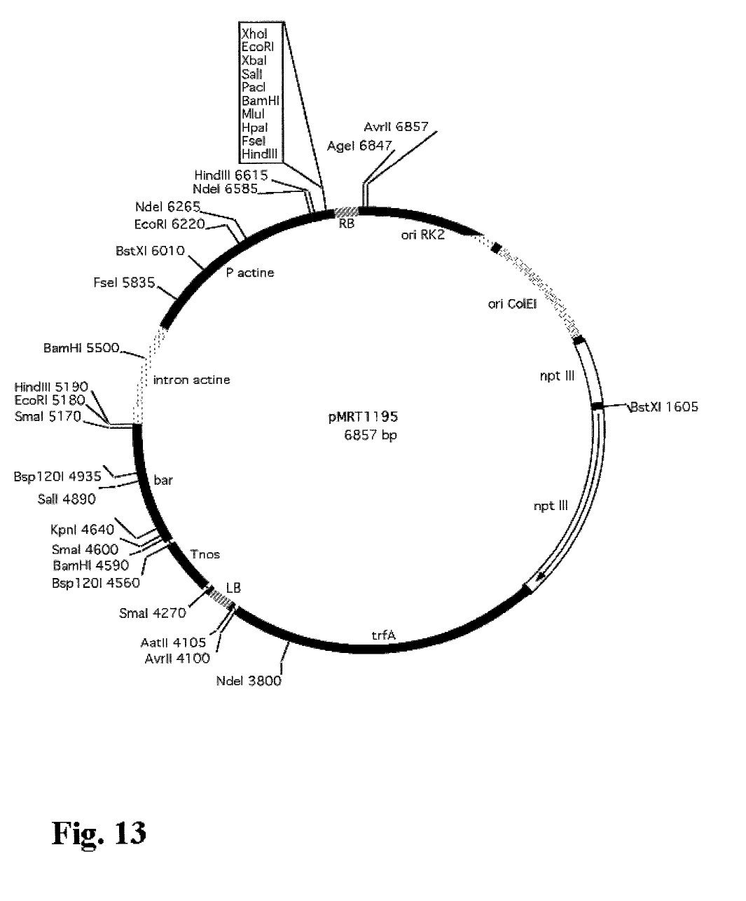

The ligation by PCR reaction was carried out as described earlier in 7, with 20 ng of dephosphorylated digested pMRT1191 plasmid and 100 ng of digested and treated insert DNA fragments. The bacteria, Escherichia coli DH5a, which had previously been made competent, were transformed. The plasmid DNA of the clones obtained, selected on LB medium supplemented with kanamycin (50 mg/l), was extracted by the alkaline lysis method and verified by enzymatic digestions and sequencing. The resultant plasmid was called pMRT1195. It is represented in FIG. 13 and its complete sequence SEQ.ID13 is given in the sequence listing.

10.3. Obtaining pMRT1202

The plasmid pMRT1202 (5614 bp) differs from pMRT1195 by the replacement of the sequence “Pact-IA” by the nopaline synthase (Pnos) promoter of Agrobacterium tumefaciens isolated from pMRT1121 produced in Escherichia coli strain SCS110.

The pMRT1195 vector fragment (2 μg) was digested by PstI, purified with the aid of the <<Concert Rapid PCR Purification System>> kit, recovered in 98 μl of H₂O, treated by the action of 6 units of T4 DNA polymerase (New England Biolabs) in a reaction medium of 120 μl in the presence of 12 μl of T4 DNA polymerase×10 buffer, 4 μl of 10 mM dNTPs and 6 μl of BSA at 1 mg/ml. The reaction was carried out at 37° C. for 30 min. The vector fragment was then purified with the aid of the <<Concert Rapid PCR Purification System>> kit, recovered in 50 μl of H₂O, digested by HindIII, isolated by 0.8% agarose gel electrophoresis, purified with the aid of the <<Concert Rapid PCR Purification System>> kit, and recovered in 98 μl of H₂O. After that, digested and treated pMRT1195 was dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, purified with the aid of the <<Concert Rapid PCR Purification System>> kit, and recovered in 50 μl of H₂O.

The insert DNA fragments corresponding to “Pnos” were obtained by BspEI digestion of pMRT1121, purification with the aid of the <<Concert Rapid PCR Purification System>> kit, recovery in 98 μl of H₂O, treatment with 20 units of the Klenow fragment (New England Biolabs) in the presence of 12 μl of 500 mM Tris-HCl, pH 7.5-500 mM MgCl2, 6 μl of 1M dithiothreitol and 6 μl of each of the 10 mM dNTPs in a reaction volume of 120 μl at 37° C. for 30 min., purification with the aid of the <<Concert Rapid PCR Purification System>> kit, recovery in 50 μl of H₂O, and digestion by HindIII. The insert DNA fragments (219 bp) carrying Pnos (209 bp) were then isolated by 2% agarose gel electrophoresis, purified with the aid of the <<Concert Rapid PCR Purification System>> kit, and recovered in 50 μl of H₂O.

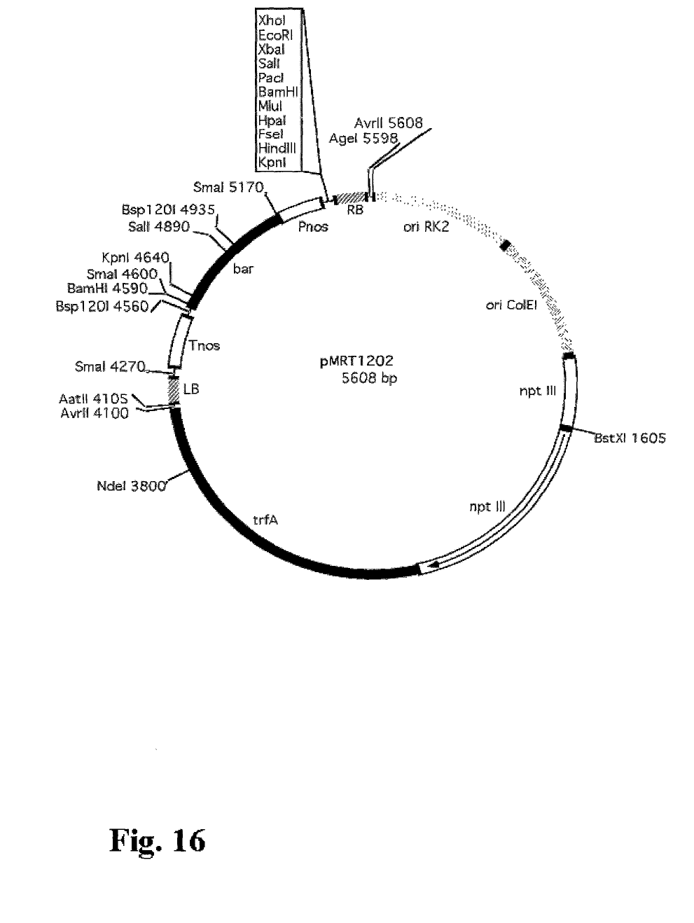

The ligation by PCR reaction was carried out as described earlier in 7, with 48 ng of dephosphorylated digested pMRT1195 plasmid and 56 ng of digested and treated insert DNA fragments. The bacteria, Escherichia coli DH5, which had previously been made competent, were transformed. The plasmid DNA of the clones obtained, selected on LB medium supplemented with kanamycin (50 mg/l), was extracted by the alkaline lysis method and verified by enzymatic digestions and sequencing. The resultant plasmid was called pMRT1202. It is represented in FIG. 16 and its complete sequence SEQ.ID16 is given in the sequence listing.

10.4. Obtaining pMRT1212

The plasmid pMRT1212 (8987 bp) differs from pMRT1206 by the replacement of the nptII gene by the bar gene.

To do this, the pMRT1206 vector fragment was obtained by digestion by Bsu36I of 2 μg of pMRT1206, purification with the aid of the <<Concert Rapid PCR Purification System>> kit, recovery in 50 μl of H₂O and digestion by AatII. The digestion by Bsu36I and AatII allowed deletion of the vector of the fragment corresponding to <<part Pnos nptII Tnos LB>>. The digested vector fragment was then subjected to 1% agarose gel electrophoresis, purified with the aid of the <<Concert Rapid PCR Purification System>> kit and recovered in 98 μl of H₂O. This digested and treated vector fragment was then dephosphorylated with 50 units of calf intestinal alkaline phosphatase (New England Biolabs) in a final reaction medium of 120 μl in the presence of 12 μl of 3×10 buffer (New England Biolabs) at 37° C. for 1 hour, purified with the aid of the <<Concert Rapid PCR Purification System>> kit and recovered in 50 μl of H₂O.

The insert DNA fragments corresponding to <<part Pnos bar Tnos LB>> (1.2 kbp) were obtained by digestion by Bsu36I of 2 μg of pMRT1202, purification with the aid of the Concert Rapid PCR Purification System kit, recovery in 40 μl of H₂O, digestion by AatII, isolation by 1% agarose gel electrophoresis, purification with the aid of the Concert Rapid PCR Purification System kit and recovery in 50 μl of H₂O.

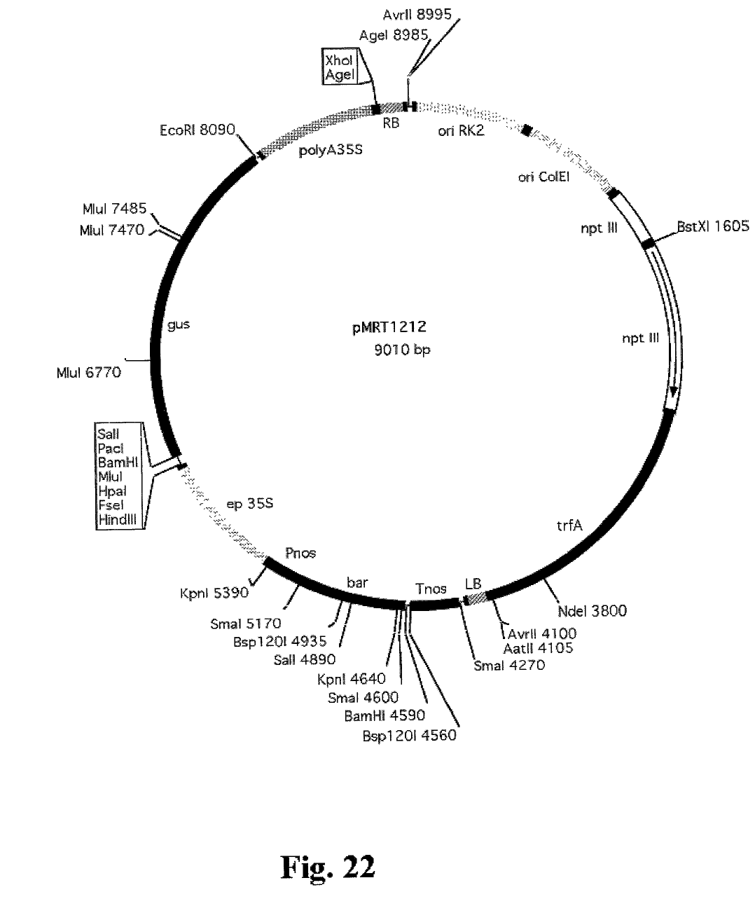

The ligation by PCR reaction was carried out as described earlier in 7, with 100 ng of dephosphorylated digested pMRT1206 plasmid and 50 ng of digested insert DNA fragments. The bacteria, Escherichia coli DH5, which had previously been made competent, were transformed. The plasmid DNA of the clones obtained, selected on LB medium supplemented with kanamycin (50 mg/l), was extracted by the alkaline lysis method and verified by enzymatic digestions and sequencing. The resultant plasmid was called pMRT1212. It is represented in FIG. 22 and its complete sequence SEQ.ID22 is given in the sequence listing.

11. Evaluation of the Synthetic Vectors Produced in the Bacteria

The yield of plasmids obtained from the bacteria Escherichia coli strain DH5 was determined.

I Non-Binary Synthetic Plasmids

Primary cultures of the recombinant bacteria possessing the plasmids pMRT1105, pMRT1106 or pMRT1105-ori ColE1 were prepared from 500 ml of each of the respective “glycerol-stocks” in 10 ml of LB medium supplemented with kanamycin at 50 mg/l, at 37° C. for 16 hours. After that, 500 ml of each of the primary cultures was cultured in 100 ml of LB supplemented with kanamycin at 50 mg/l at 37° C. for 23 hours. For pMRT1106, two cultures were prepared, one without addition of chloramphenicol and the other with addition of chloramphenicol at 40 mg/l after 7 hours of culture. The cultures were then centrifuged at 5000 g for 10 min. The plasmid DNAs were extracted with the aid of the “QIAFilter Plasmid Midi kit” (QIAGEN), in accordance with the manufacturer's recommendations, and quantitatively determined with the spectrophotometer. The quantities of plasmids obtained were determined as 10 mg, 50.6 mg, 86.2 mg and 9.8 mg respectively for pMRT1105, pMRT1106, pMRT1106 treated with chloramphenicol, and pMRT1105 ori ColE1 (plasmid pMRT1105 in which ori ColE1 was inserted in the reverse orientation to that of pMRT1106). These results show that the synthetic plasmids are replicated in Escherichia coli, the presence of the origin of replication “ori ColE1” in the right orientation (NdeI site of “ori ColE1” near “ori RK2”) allows the yield to be increased by a factor of 5, whereas no effect is shown for the reverse orientation of “ori ColE1”, treatment with chloramphenicol contributes an additional factor of 1.6.

11.2. Synthetic Binary Plasmids.

The synthetic binary plasmids pMRT1118 and pMRT1119 are evaluated relative to the controls pBin19 and pBIOC4. The plasmid pBIOC4 differs from pGA492 (An,1986) by the deletion of virtually all the coding sequence of the pGA492 cat gene and by the conversion of the HindIII site into an EcoRI site. It was obtained by double digestion by SacI and ScaI followed by the action of the enzyme T4 DNA polymerase (New England Biolabs) and ligation of the modified plasmid (20 ng) in a reaction medium of 10 μl in the presence of 1 μl of T4 DNA ligase×10 buffer (Amersham) and 2.5 units of T4 DNA ligase (Amersham) at 14° C. for 16 hours. The bacteria, Escherichia coli strain DH5, which had previously been made competent, were transformed (Hanahan, 1983). The plasmid DNA of the clones obtained, selected on tetracycline at 12 mg/l, was extracted by the alkaline lysis method (Birnboim and Doly, 1979) and analysed by enzymatic digestions. The HindIII site of the plasmid DNA of the clone selected were then modified into an EcoRI site with the aid of a phosphorylated HindIII-EcoRI adaptor (Stratagene). To do this, 500 ng of plasmid DNA of the clone selected were digested by HindIII, dephosphorylated by the enzyme calf intestinal alkaline phosphatase (Boehringer Mannheim) in accordance with the manufacturer's recommendations and coprecipitated in the presence of 1500 ng of HindIII-EcoRI adaptor DNA, 0.1 volume of 3M sodium acetate, pH 4.8, and 2.5 volumes of absolute ethanol at −80° C. for 30 min. After centrifugation at 12000 g for 30 min., the precipitated DNA was washed with 70% ethanol, dried, recovered in 8 μl of H₂O, held at 65° C. for 10 min., then ligated as described earlier. After inactivation of the T4 DNA ligase at 65° C. for 10 min., the ligation reaction mixture was digested by EcoRI, purified by 0.8% agarose gel electrophoresis, electroeluted, precipitated with absolute ethanol, centrifuged at 12000 g for 30 min., washed with 70% ethanol, dried, then ligated and introduced into Escherichia coli strain DH5, and treated as described earlier.

Primary cultures of the recombinant bacteria possessing the plasmids mentioned above were prepared from 500 ml of each of the respective “glycerol-stocks” in 10 ml of LB medium supplemented with kanamycin at 50 mg/l, except for pBIOC4, which was selected on tetracycline at 12 mg/l, at 37° C. for 16 hours. After that, 250 ml of each of the primary cultures were cultured in 100 ml of LB supplemented with the appropriate antibiotic at 37° C. for 23 hours. The cultures were then centrifuged at 5000 g for 10 min. The plasmid DNAs were extracted with the aid of the “QIAFilter Plasmid Midi kit” (QIAGEN) in accordance with the manufacturer's recommendations and quantitatively determined with the spectrophotometer. The quantities of plasmids obtained were determined as 94.2 mg, 113 mg, 37.2 mg and 45.6 mg respectively for pMRT1118, pMRT1119, pBin19 and pBIOC4.

These results show that the synthetic binary plasmids are replicated in Escherichia coli, and that the yield of pMRT1119 is increased by the factors 3 and 2.5 respectively, relative to the pBin19 and pBIOC4 controls.

Furthermore, these synthetic plasmids are replicated in Agrobacterium tumefaciens strain LBA4404.

12. Evaluation of the Synthetic Plasmids by Transgenesis

12.1. Transformation of Tobacco

12.1.1. Stable Transformation of Tobacco

The transformation of tobacco (Nicotiana tabacum L. var. PBD6) was carried out by infecting foliar discs isolated from 6-week-old tobacco seedlings in vitro with recombinant agrobacteria in accordance with the method described by Horsch et al. (1985).

All the in vitro cultures are prepared in a controlled environment chamber in which the light intensity is 200 μE.m-2.s-1, the photoperiod 16 hours light/8 hours darkness, and the temperature 25° C.

Except for the initial coculture stage, the regeneration, development and rooting stages were performed on different selective media supplemented with a bacteriostat, namely augmentin at 400 mg/l, and a selective agent, namely kanamycin at 200 or 100 mg/l.

The different stages and the media used are as follows:

After primary culture of the agrobacteria in 5 ml of 2YT medium (bacto-tryptone 10 g/l, yeast extract 5 g/l, NaCl 5 g/l, pH 7.2) supplemented with CaCl₂, with a final concentration of 6 mM, and with appropriate antibiotics, at 28° C. for 48 hours, a culture is performed in 10 ml of 2YT medium, supplemented with CaCl₂ and antibiotics, at 28° C. for a period of one night. The culture is then centrifuged at 3000 g for 10 min. and the bacteria are resuspended in 10 ml of liquid MS30 (M0404 at 4.4 g/l, marketed by SIGMA, supplemented with sucrose, 30 g/l, pH 5.7).

The coculture is performed by placing the foliar explants cut from the leaves of the seedlings in vitro, about 1 cm², in contact with the suspension of agrobacteria, diluted 1/10, in liquid MS30 for 20 min. The explants so treated are then rapidly dried on filter paper and placed on a solid coculture medium (CM) (MS30, Benzyl Amino Purine (BAP) 1 mg/l, Indole-3 Acetic Acid (ANA) at 0.1 mg/l, agar at 8 g/l) for 48 hours in the controlled environment chamber.

The treated explants are then placed on a solid regeneration medium (solid CM, augmentin 400 at mg/l, kanamycin at 200 mg/l). The explants are pricked out on the same medium after 2 weeks.

After 2 weeks the shoots are pricked out on a solid development medium (M0404 at 4.4 g/l, marketed by SIGMA, supplemented with sucrose 20 g/l, pH 5.7 (liquid MS20), augmentin at 400 mg/l, kanamycin at 100 mg/l, agar at 8 g/l).

After 2 weeks, the transformed seedlings are pricked out on a solid rooting medium identical to the development medium. Rooting takes 2 to 3 weeks, after which the seedlings are removed to the growth chamber in Giffy pots for 10 days (photoperiod 16 hours light/8 hours darkness, 23° C., 70% relative humidity), then placed in the greenhouse.

12.1.2. Evaluation of the Synthetic Plasmids Used in Transformation of Tobacco

The recombinant agrobacteria (Agrobacterium tumefaciens strain LBA4404) containing the synthetic binary plasmid pMRT1118 or the control binary plasmid pBin19 were used to transform the tobacco Nicotiana tabacum L. var. PBD6.

The results showed that the number and growth of the seedlings which develop in the presence of the selective agent (kanamycin) are similar for the two binary plasmids tested. These observations indicate that the synthetic binary plasmid is perfectly functional in transgenesis of tobacco.

12.2. Transformation of Maize

12.2.1. Genetic Transformation of Maize Calli

The genetic transformation of maize, whatever method is employed (electroporation, Agrobacterium, microfibres, particle gun), generally requires the use of cells that are undifferentiated in rapid divisions, having retained the ability to regenerate whole plants. Cells of this type make up the friable embryogenic callus (called Type II) of maize.

These calli were obtained from immature embryos of genotype HI, II or (A188×B73) in accordance with the method and on the media described by Armstrong (Maize handbook, 1994, Freeling M and Walbot V (eds), pp 665-671). The calli so obtained were multiplied and maintained by successive subcultures every 2 weeks on the starting medium.

Seedlings were regenerated from these calli by modifying the hormonal and osmotic equilibrium of the cells in accordance with the method described by Vain et al. (1989). These plants were then hardened off in the greenhouse, where they can be crossed or selfed.

12.2.2. Genetic Transformation of Maize with the Particle Gun

The production and regeneration of the cell lines needed for transformation are described in 12.2.1.

The procedure which uses the particle gun for genetic transformation is described by Finer et al. (1992). The target cells are callus fragments with a surface area of 10 to 20 mm². These fragments were placed on a starting medium supplemented with 0.2 M mannitol and 0.2 M sorbitol for 4 hours before bombardment.

The tungsten particles (M10) and transformation plasmids were coprecipitated in accordance with the protocol described by Klein (1987). The particles thus coated were projected towards the target cells by means of the particle gun in accordance with the protocol of Finer et al. (1992).

The bombarded calli were placed in darkness at 27° C. After 24 hours, the first subculturing took place, followed by subculturing every 2 weeks for 3 months on a starting medium to which the appropriate selective agent to select only the transformed calli had been added. These calli were then grown in the presence of the selective agent in order to regenerate transformed seedlings, as described in 12.2.1. The seedlings obtained were hardened off and transferred to the greenhouse, where they can be crossed or selfed.

12.2.3. Genetic Transformation of Maize by Agrobacterium tumefaciens

The technique used is described by Ishida et al. (1996). Immature embryos 1.0 to 1.2 mm in length (9 to 14 days after pollination) were washed in the LS-inf medium, then immersed in the suspension of agrobacteria, prepared as described by Ishida et al. (1996), vortexed for 30 sec., and incubated at ambient temperature for 5 min. The immature embryos so treated were cultured on LS-AS medium in darkness at 25° C. for 3 days, then transferred to LSD 1.5 medium supplemented with phosphinothricin at 5 mg/l and cefotaxime at 250 mg/l, in darkness at 25° C. for 2 weeks, and finally placed on LSD 1.5 medium supplemented with phosphinothricin at 10 mg/l and cefotaxime at 250 mg/l, in darkness at 25° C. for 3 weeks. The Type I calli thus generated were isolated, fragmented and transferred to LSD 1.5 medium supplemented with phosphinothricin at 10 mg/l and cefotaxime at 250 mg/l, in darkness at 25° C. for 3 weeks. The Type I calli which proliferated were then isolated and placed on LSZ medium supplemented with phosphinothricin at 5 mg/l and cefotaxime at 250 mg/l, subjected to a photoperiod of 16 hours light/8 hours darkness at 25° C. for 2 to 3 weeks. The regenerated seedlings were then transferred to ½ LSF medium subjected to a photoperiod of 16 hours light/8 hours darkness at 25° C. for 1 to 2 weeks, and then transferred to the growth chamber and greenhouse.

13. Influence of the Vector Backbone on Gene Expression

Stably transformed tobacco plants were obtained for the synthetic binary plasmids pMRT1204 bearing the wild-type nptII gene (16 plants) and pMRT1206 bearing the mutated nptII gene (12 plants), and also for the control vector pBI121 sold by Clontech which corresponds to plasmid pBIN19 bearing the wild-type nptII gene and containing the expression cassette “P35S-uidA-polyA nos” (11 plants).

The influence of the synthetic vector backbone and that of the control was evaluated by ELISA analysis of protein extracts using the NPTII ELISA kit sold by 5 Prime→3 Prime. The analysis enabled a quantification of the NPTII protein produced (in micrograms, μg) with respect to the amount of soluble extracted protein(in milligrams, mg).

The proteins were extracted in “Tris-HCl 25 mM pH7.8; Phenylmethylsulfonylfluoride 1 mM” buffer from tobacco leaves ground in liquid nitrogen. After centrifugation for 10 minutes at 10000 g and 4° C., the extracted soluble proteins contained in the supernatant were measured according to Bradford's method (A rapid and sensitive method for the detection of microgram quantities of proteins utilizing the principle of protein-dye binding. Anal. Biochem. (1976)72, 248-254).

The antibodies used for ELISA quantification were rabbit polyclonal antibodies specifically recognizing the coating NPTII protein and biotinylated anti-NPTII anitbodies for the detection of the NPTII protein.

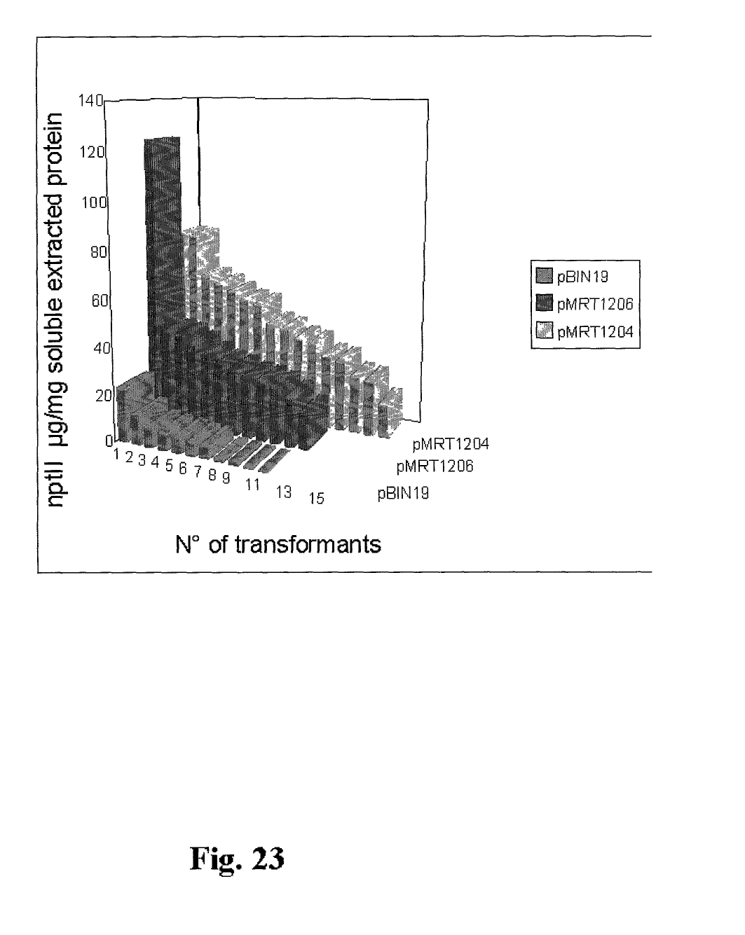

The results of the analysis shown in FIG. 23 indicate that:

for the wild type nptII gene cassette and different plasmid backbones, the quantities of NPTII obtained with the synthetic binary plasmid pMRT1204 are greater by a factor of 6.7 than those obtained with the original pBIN19 binary plasmid, which demonstrates the efficiency of the synthetic vector.

for an identical plasmid backbone, i.e. synthetic binary plasmids pMRT1204 and pMRT1206, but with different nptII genes, i.e. Wild type and mutated nptII, the quantities of NPTII obtained in the wild type gene differ from those obtained for the mutated nptII gene by a factor 1.2.

14. Synthesis of pMRT1334.

The plasmid pMRT1334 (9688 bp) was obtained by replacing the nptII expression cassette of pMRT1206 by the nptII expression cassette of pBIN19.

The vector fragment derived from pMRT1206 was obtained by digestion of 10 μg of pMRT1206 with KpnI, purified using a “Concert Rapid PCR Purification System” kit, and taken up in 50 μl of water. Then the digested pMRT1206 was subjected to the action of 6 units of T4 DNA polymerase (New England Biolabs) in a reaction mixture of 120 μl in the presence of 12 μl of T4 10× DNA polymerase buffer, 4 μl of 10 mM dNTP and 6 μl of 1 mg/ml BSA. The reaction was carried out at 37° C. for 30 minutes. The digested and thus treated pMRT1206 vector was purified with a “Concert Rapid PCR Purification System” kit, taken up in 50 μl of water, then digested with AflII. The vector fragment derived from pMRT1206 was isolated by electrophoresis on a 1% agarose gel, purified with a “Concert Rapid PCR Purification System” and taken up in 50 μl of water.

The DNA insert fragment DraI-AflII (1.5 kbp), corresponding to the nptII expression cassette was obtained by digestion of 9 μg of pBIN19 plasmid with DraI and AflII. Then, the fragment was subjected to electrophoresis on a 1% agarose gel in TEB buffer, purified using a “Concert Rapid PCR Purification System”, and taken up in 50 μl of water.

The PCR ligation reaction was carried out using 100 ng of the plamsid fragment derived from pMRT1206 and 100 ng of the DNA insert fragment DraI-AflII in a reaction mixture of 20 μl in the presence of 2 μl of T4 10× DNA ligase buffer (Epicentre Technologies), 2 μl 2.5 mM ATP and 4 units of T4 DNA ligase (Epicentre Technologies). The reaction consists of 180 cycles each including 2 steps, the first at 10° C. for 30 seconds and the second at 30° C. for 30 seconds in a “GeneAmp PCR System 9700” thermocycler.

Previously prepared competent Escherichia coli DH5 bacteria, were transformed (Hanahan, 1983). The plamsid DNA of the obtained clones, selected on LB media supplemented with kanamycin (50 mg/l), was extracted according to the alakline lysis method and verified by enzymatic digestion and sequencing. The resulting plasmid was designated pMRT1334 (FIG. 24). Its complete sequence is given under SEQ.ID50 in the sequence listing.

15. Synthesis of pMRT1335

The plasmid pMRT1335 (15208 bp) is a control vector and results from the insertion of the expression cassette “ep35S-gus (uidA)-polyA35S” isolated from pMRT1206 into pBIN19.

13 μg of pBIN19 plasmid were digested with EcoRI, purified using a “Concert Rapid PCR Purification System”, and taken up in 50 μl water. Then the previously digested pBIN19 was subjected to the action of 20 units of Klenow fragment (New England Biolabs) in the presence of 12 μl 500 mM pH7.5 Tris-HCl 500 mM MgCl2, 6 μl of 1M dithiothreitol, 6 μl each of 10 mM dNTP in a reaction volume of 120 μl at 37° C. for 60 minutes. The digested pBIN19 vector was purified using a “Concert Rapid PCR Purification System”, taken up in 50 μl water, then digested with KpnI. The vector fragment thus produced was isolated by electrophoresis on a 1% agarose gel, and purified using a “Concert Rapid PCR Purification System” and taken up in 50 μl of water.

The DNA insert fragment corresponding to the “ep35S-gus-polyA35S” (3.5 kbp) expression cassette was obtained by digestion of 10 μg of plasmid pMRT1206 with XhoI, purified using a “Concert Rapid PCR Purification System”, and taken up in 50 μl water, followed by the action of 20 units of Klenow fragment (New England Biolabs) in the presence of 12 μl of 500 mM pH7.5 Tris-HCl 500 mM MgCl2, 6 μl 1M dithiothreitol, 6 μl each of 10 mM dNTP in a reaction volume of 120 μl at 37° C. for 60 minutes, then purified using a “Concert Rapid PCR Purification System”, taken up in 50 μl water, and finally digested with KpnI. The DNA insert fragment thus produced was isolated by electrophoresis on a 1% agaros gel in TEB buffer, and purified using a “Concert Rapid PCR Purification System”, and taken up in 50 μl water.

The PCR ligation reaction was carried out with 100 ng of pBIN19 treated and 100 ng of DNA insert fragment as prepared previously in a reaction volume of 20 μl in the presence of 2 μl of T4 10× DNA ligase buffer (Epicentre Technologies), 2 μl 2.5 mM ATP and 4 units T4 DNA ligase (Epicentre Technologies). The reaction consists of 180 cycles each including 2 steps, the first at 10° C. for 30 seconds and the second at 30° C. for 30 seconds in a “GeneAmp PCR System 9700” thermocycler.

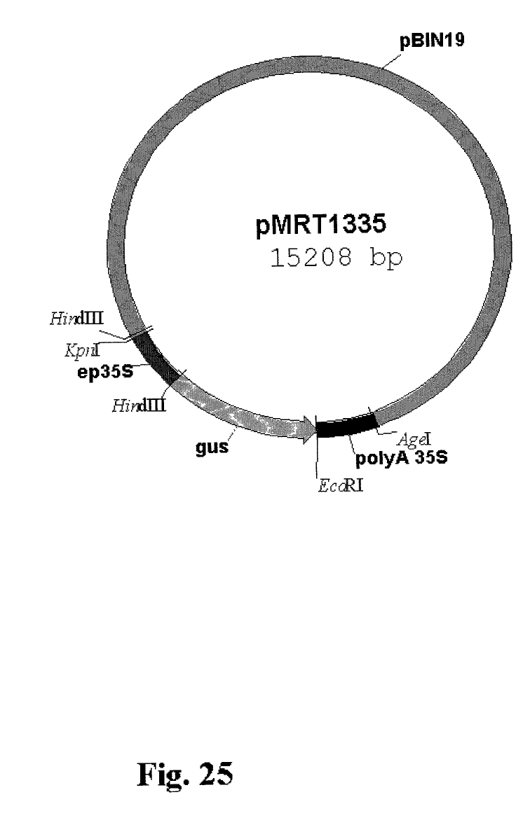

Previously prepared competent Escherichia coli DH5 bacteria, were transformed (Hanahan, 1983). The plamsid DNA of the obtained clones, selected on LB media supplemented with kanamycin (50 mg/l), was extracted according to the alakline lysis method and verified by enzymatic digestion and sequencing. The resulting plasmid was designated pMRT1335 (FIG. 25). Its complete sequence is given under SEQ.ID51 in the sequence listing.

16. Synthesis of pMRT1336

The plamsid pMRT1336 (9285 bp) results from the insertion into pMRT1196 of the promoter MPr1165 (610 bp) isolated from plasmid pMRT1322 as described in PCT patent application PCT/IB00/00370, and incorporated into the present description by reference thereto for that relevant part.

10 μg of pMRT1196 plasmid were digested with KpnI, purified using a “Concert Rapid PCR Purification System”, takn up in in 50 μl water, and redigested with HpaI. The thus treated vector fragment was subjected to electrophoresis on a 1% agarose gel, and purified using a “Concert Rapid PCR Purification System” and then taken up in 50 μl water.

The DNA insert fragment bearing the promoter MPr1165 (0.5 kbp) was obtained from 10 μg of pMRT1322 plasmid digested with KpnI, purified using a “Concert Rapid PCR Purification System” and taken up in 50 μl water, and then redigested with HpaI. The DNA insert fragment thus produced was subjected to gel electrophoresis on a 1% agarose gel in TEB buffer, purified using a “Concert Rapid PCR Purification System”, and taken up in 50 μl water.

The PCR ligation reaction was carried out using 100 ng of vector fragment and 100 ng DNA insert fragment bearing the promoter MPr1165 as prepared above in a reaction volume of 20 μl in the presence of de 2 μl T4 10× DNA ligase buffer (Epicentre Technologies), 2 μl 2.5 mM ATP and 4 units T4 DNA ligase (Epicentre Technologies). The reaction consists of 180 cycles each including 2 steps, the first at 10° C. for 30 seconds and the second at 30° C. for 30 seconds in a “GeneAmp PCR System 9700” thermocycler.

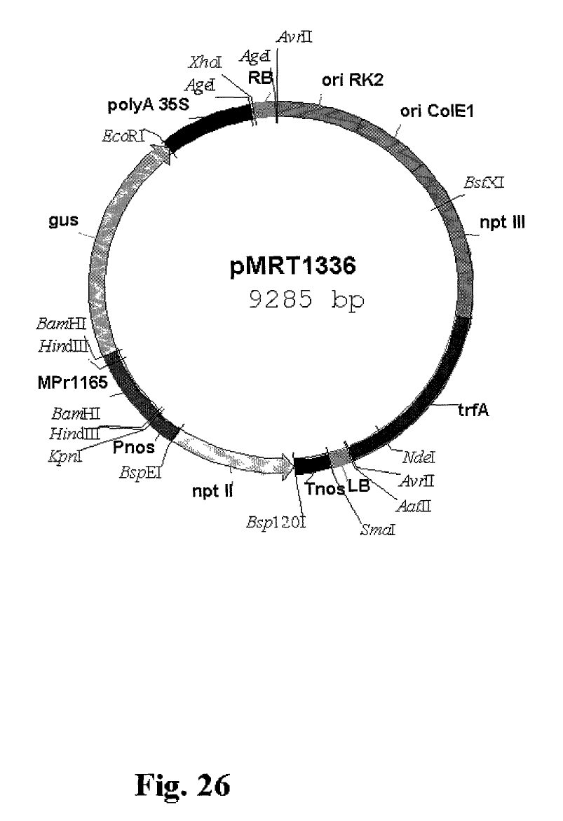

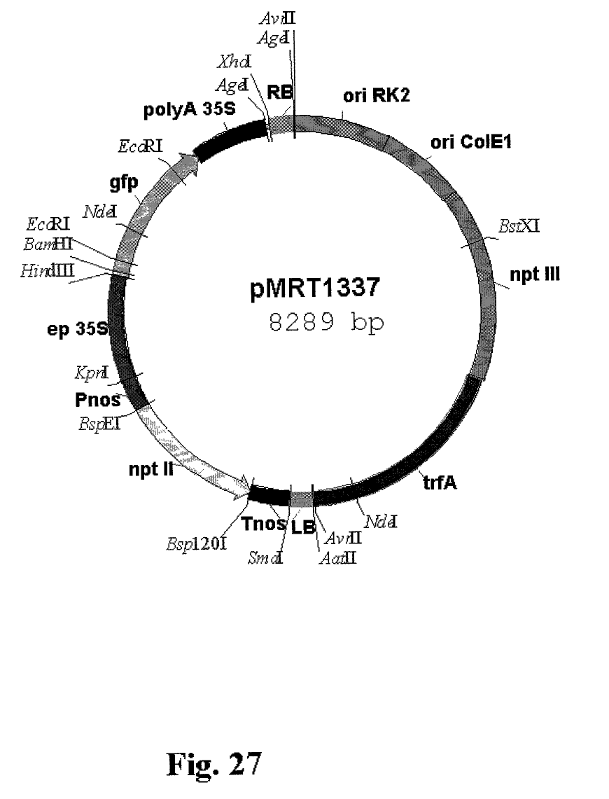

Previously prepared competent Escherichia coli DH5 bacteria, were transformed (Hanahan, 1983). The plamsid DNA of the obtained clones, selected on LB media supplemented with kanamycin (50 mg/l), was extracted according to the alakline lysis method and verified by enzymatic digestion and sequencing. The resulting plasmid was designated pMRT1336 (FIG. 26). Its complete sequence is given in the sequence listing under SEQ.ID52.

Plasmid pMRT1322 was obtained by the insertion of promoter MPr1165 into pMRT1176. 10 μg of pMRT1176 plasmid was digested with XbaI and EcoRI, purified using a “Concert Rapid PCR Purification System” kit, taken up in 50 μl water, dephosphorylated with 50 units of calf intestine alkaline phosphatase (New England Biolabs) in a final reaction volume of 120 μl in the presence 12 μl of 3×10 buffer (New England Biolabs) at 37° C. for 1 hour, and purified using a “Concert Rapid PCR Purification System” kit, and then taken up in 50 μl water. The DNA insert fragment corresponding to promoter MPr1165 was obtained by PCR amplification from 10 ng of pMRT1240 matrix DNA usingn 20 pmoles each of two oligodesoxynucleotides, 5′ AGCTCTAGAGCTGCCTGCAGCACTAGTATCC 3′(SEQ.ID53) bearing the site XbaI and 5′ CGGAATTCGGCCTCTAGGTTGTTGTGTTG 3′(SEQ.ID54) bearing the site EcoRI, using the enzyme Platinum Taq polymerase High Fidelity (GIBCO BRL Life Technologies) and following the suppliers instructions. The PCR amplification reaction was carried out in a “GeneAmp PCR System 9700” thermocycler. After denaturation at 94° C. for 2 minutes, the DNA was subjected to 25 cycles, each including denaturation steps at 94° C. for 45 seconds, hybridisation at 55° C. for 45 seconds and elongation at 68° C. for 45 seconds. On the final cycle the elongation was continued at 68° C. for 3 minutes. The PCR product obtained was isolated by electrophoresis on a 1.5% agarose gel in TBE buffer and purified using a “Concert Rapid PCR Purification System” kit, and then taken up in 50 μl water. The PCR product was then digested with XbaI and EcoRI, purified using a “Concert Rapid PCR Purification System” kit, and taken up in 50 μl water. The PCR ligation reaction was carried out as described previously with 80 ng of pMRT1176 vector fragment and 110 ng of DNA insert fragment bearing the MPr1165 promoter. Previously prepared competent Escherichia coli DH5 bacteria, were transformed (Hanahan, 1983). The plamsid DNA of the obtained clones, selected on LB media supplemented with kanamycin (50 mg/l), was extracted according to the alakline lysis method and verified by enzymatic digestion and sequencing. The resulting plasmid was designated pMRT1322.

Plasmid pMRT1240 was obtained by insertion of a DNA fragment into plasmid pMRT1234, by treatment with Klenow fragment (New England Biolabs) according to the supplier's instructions of the HindIII sites and BamHI. The DNA insert fragment was the L5 promoter fragment obtained by double PstI digestion, and BamHI digestion, followed by the action of T4 DNA polymerase (New England Biolabs) according to the supplier's instructions, of plasmid pMRT1165, as described in PCT patent application PCT/IB00/00370, and incorporated into the present description by reference thereto for that relevant part.

Plasmid pMRT1234 was obtained by the insertion at the EcoRI site of pMRT1175 of a DNA insert fragment EcoRI corresponding to the cDNA coding for human lacotferrin isolated from the plamsid pHMWG-IA-PSSp-Lf, as described in previously published patent application WO98/50543.

17. Synthesis of pMRT1337

Plasmid pMRT1337 (8289 bp) was obtained from the insertion, into pMRT1205, of the gfp gene isolated from pBINm-gfp5-ER and described in Haseloff J et Siemering K R. 1998, “The uses of green fluorescent protein in plants”, in Green fluorescent protein: Strategies, applications and protocols (Chalfie M et Kain S, eds, Wiley, pp 191-220).

10 μg of plasmid pMRT1205 was digested with EcoRI, purified using a “Concert Rapid PCR Purification System>> kit, taken up in 50 μl water, subjected to the action of 20 units of Klenow fragment (New England Biolabs) in the presence of 12 μl of 500 mM pH7.5 Tris-HCl 500 mM MgCl2, 6 μl 1M dithiothreitol, 6 μl each of 10 mM dNTP in a reaction volume of 120 μl at 37° C. for 60 minutes, purified using a “Concert Rapid PCR Purification System” kit, taken up in 50 μl water, and then digested with BamHI. The thus treated vector fragment was subjected to electrophoresis on 1% agarose gel, purified using a “Concert Rapid PCR Purification System” kit and taken up in 50 μl water.

The DNA insert fragment bearing the gfp gene (0.7 kbp) was obtained from 10 μg of plasmid pBINm-gfp5-ER digested with SacI, purified using a “Concert Rapid PCR Purification System” kit, taken up in 50 μl water, and subjected to the action of 6 units of T4 DNA polymerase (New England Biolabs) in a reaction volume of 120 μl in the presence of 12 μl T4 10× DNA polymerase buffer, 4 μl 10 mM dNTP and 6 μl 1 mg/ml BSA. The reaction was carried out at 37° C. for 30 minutes. The thus treated plasmid pBINm-gfp5-ER was then purified using a “Concert Rapid PCR Purification System” kit, taken up in 50 μl water, and digested with BamHI. The DNA insert fragment thus produced was subjected to gel electrophoresis on 1% gel agarose in TEB buffer, purified using a “Concert Rapid PCR Purification System” kit and taken up in 50 μl in water.

The PCR ligation reaction was carried out with 100 ng of pMRT1205 vector fragment and 100 ng DNA insert fragment bearing the gfp gene as prepared above in a reaction volume of 20 μl in the presence of 2 μl T4 10× DNA ligase buffer (Epicentre Technologies), 2 μl 2.5 mM ATP and 4 units T4 DNA ligase (Epicentre Technologies). The reaction comprised 180 cycles each including two steps, the first at 10° C. for 30 seconds, the second at 30° C. for 30 seconds, in a “GeneAmp PCR System 9700” thermocycler.

Previously prepared competent Escherichia coli DH5 bacteria, were transformed (Hanahan, 1983). The plamsid DNA of the obtained clones, selected on LB media supplemented with kanamycin (50 mg/l), was extracted according to the alakline lysis method and verified by enzymatic digestion and sequencing. The resulting plasmid was designated pMRT1337 (FIG. 27). Its complete sequence is given in the sequence listing under SEQ.ID55.

18. Synthesis of pMRT1341

The plasmid pMRT1341 (14108 bp) results from the replacement of the “ep35S-gus-polyA35S” expression cassette from pMRT1335 by the expression cassette “ep35S-gfp-polyA35S” isolated from pMRT1337.

10 μg of plasmid pMRT1335 was digested by AgeI and KpnI. The thus digested vector fragment was isolated by electrophoresis on a 0.8% agarose gel, purified using a “Concert Rapid PCR Purification System” kit and then take up in 50 μl water.

The DNA insert fragment corresponding to the expression cassette “ep35S-gfp-polyA35S” (2.4 kbp) was obtained by digesting with XhoI of 10 μg plasmid pMRT1337, purifying using a “Concert Rapid PCR Purification System” kit, taking up in 50 μl water, and the action of 20 units of Klenow fragment (New England Biolabs) in the presence of 12 μl 500 mM pH7.5 Tris-HCl 500 mM MgCl2, 6 μl 1M dithiothreitol, 6 μl each of 10 mM dNTP in a reaction volume of 120 μl at 37° C. for 30 minutes, then, purifying using a “Concert Rapid PCR Purification System” kit, taking up in 50 μl water, and finally digesting with KpnI. The thus preapred DNA insert fragment was isolated by electrophoresis on 0.8% agarose gel in TEB buffer, purified using a “Concert Rapid PCR Purification System” kit, taken up in 50 μl water, digested with AgeI, purified using a “Concert Rapid PCR Purification System” and taken up again in 50 μl water.

The PCR ligation reaction was carried out on 100 ng vector fragment derived from pMRT1335 et 100 ng DNA insert fragment as produced above in a reaction volume of 20 μl in the presence of 2 μl T4 10× DNA ligase buffer (Epicentre Technologies), 2 μl 2,5 mM ATP and 4 units of T4 DNA ligase (Epicentre Technologies). The reaction comprised 180 cycles each including 2 steps, the first at 10° C. for 30 seconds, the second at 30° C. for 30 seconds, in a “GeneAmp PCR System 9700” thermocycler.

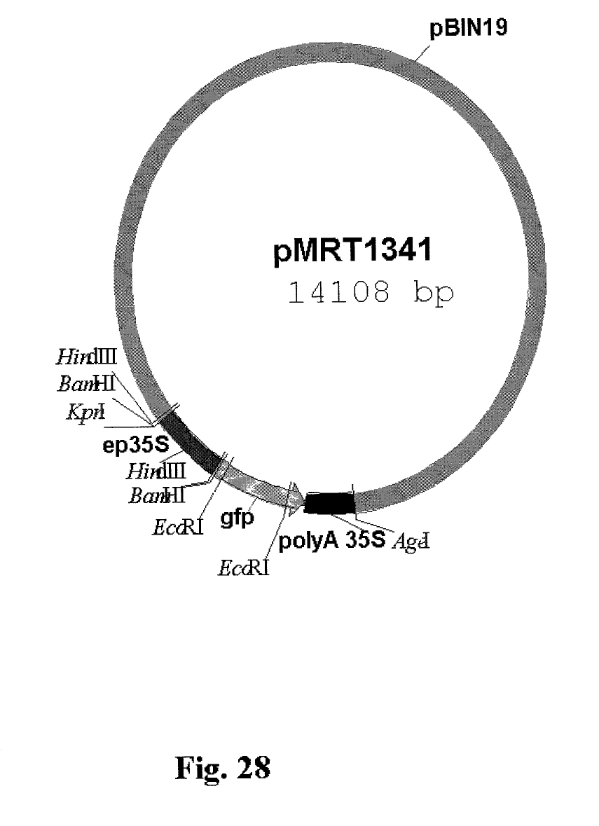

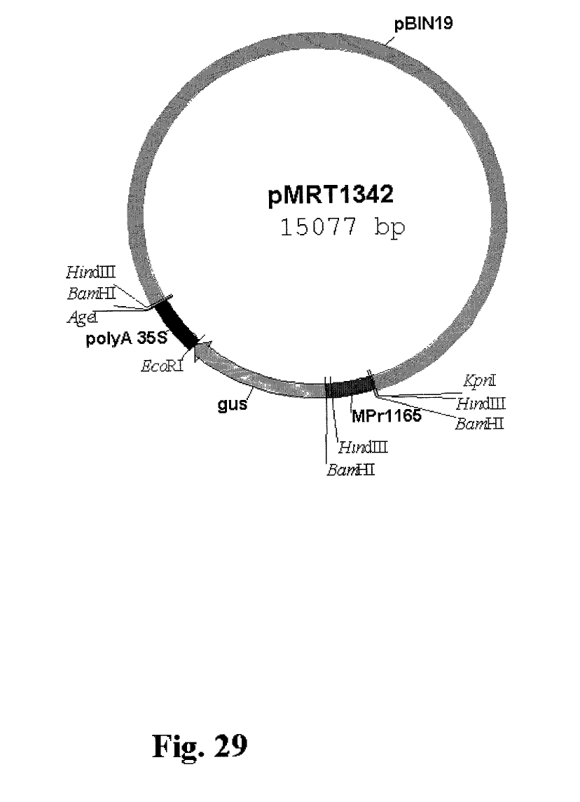

Previously prepared competent Escherichia coli DH5 bacteria, were transformed (Hanahan, 1983). The plamsid DNA of the obtained clones, selected on LB media supplemented with kanamycin (50 mg/l), was extracted according to the alakline lysis method and verified by enzymatic digestion and sequencing. The resulting plasmid was designated pMRT1341 (FIG. 28). Its complete sequence is given in the sequence listing under SEQ.ID56.

19. Synthèse de pMRT1342

Plasmid pMRT1342 (15077 bp) results from the replacement of the expression cassette “ep35S-gus-polyA35S” from pMRT1335 by the expression cassette “L5-gus-polyA35S” isolated from pMRT1336.

10 μg of plasmid pMRT1335 was digested with AgeI and KpnI. The thus digested vector fragment was isolated by electrophoresis on 0.8% agarose gel, purified using a “Concert Rapid PCR Purification System” kit and taken up in 50 μl water.

The DNA insert fragment corresponding to the expression casssette “L5-gus-polyA35S” (3.4 kbp) was obtained by digestion with XhoI of 10 μg plasmid pMRT1336, purified using a “Concert Rapid PCR Purification System” kit, taken up in 50 μl water, subjecting this to the action of 20 units Klenow fragment (New England Biolabs) in the presence of 12 μl 500 mM pH7.5 Tris-HCl 500 mM MgCl2, 6 μl 1M dithiothreitol, 6 μl each of 10 mM dNTP in a reaction volume of 120 μl at 37° C. for 30 minutes, then, purified using a “Concert Rapid PCR Purification System” kit, and taken up in 50 μl water, and finally digested with KpnI. The thus prepared DNA insert fragment was isolated by electrophoresis on a 0?8% agarose gel in TEB buffer, purified using a “Concert Rapid PCR Purification System” kit, taken up in 50 μl water, digested with AgeI, purified using a kit as above and take nup again in 50 μl water.

The PCR ligation reaction was carried out with 100 ng of vector fragment derived from pMRT1335 and 100 ng of DNA insert fragment prepared as described above in reaction mixture of 20 μl in the presence of 2 μl T4 10× DNA ligase buffer (Epicentre Technologies), 2 μl 2.5 mM ATP and 4 untis T4 DNA ligase (Epicentre Technologies). The reaction is comprised of 180 cycles each including 2 steps, the first at 10° C. for 30 seconds and the second at 30° C. for 30 seconds in a “GeneAmp PCR System 9700” thermocycler.

Previously prepared competent Escherichia coli DH5 bacteria, were transformed (Hanahan, 1983). The plamsid DNA of the obtained clones, selected on LB media supplemented with kanamycin (50 mg/l), was extracted according to the alakline lysis method and verified by enzymatic digestion and sequencing. The resulting plasmid was designated pMRT1342 (FIG. 29). Its complete sequence is given in the sequence listing under SEQ.ID57.

20. Production of Recombinant Agrobacteria

The binary plamsids were transferred into the Agrobacterium tumefaciens LBA4404 strain according to the technique described by [Holsters et al. [Holsters M., Dewaele D., Depicker A., Messenf E., Van Montagu M. et Schell J. (1978). Transfection and transformation of Agrobacterium tumefaciens. Mol. Gen. Genet. 136, 181-187.]. The plasmid DNA of the obtained clones, selected on LB media supplemented with rifampicine (50 mg/l), was extracted according to the alkaline lysis method, modified by the addition of lysozyme (25 mg/ml) in the cell resuspension buffer. The plasmid DNA obtained was analysed by enzymatic digestion. The agrobacteria clones obtained were used for plant genetic transformation.

References

An G. 1986. Development of plant promoter expression vector and their use for analysis of differential activity of nopaline synthase promoter in transformed tobacco cells. Plant Physiol. 81:86-91.

Anderson O D, Green F C, Yip R E, Halford N G, Shewry P R and Malpica-Romero J M. 1989. Nucleotide sequences of the two high-molecular-weight glutenin genes from the D-genome of a hexaploid bread wheat, Triticum aestivum L. Cheyenne. Nucl. Acids Res. 17:461-462.

Birnboim H C and Doly J. 1979. A rapid alkaline extraction procedure for screening recombinant plasmid DNA. Nucl. Acids Res. 7:1513.

Depicker A, Stachel S, Dhaese P, Zambryski P and Goodman H M. 1982. Nopaline synthase: transcript mapping and DNA sequence. J. Mol. Appl. Genet. 1:561-573.

Finer J J, Vain P, Jones M W and McMullen M D. 1992. Development of the particle inflow gun for DNA delivery to plant cells. Plant Cell Rep. 11:323-328.

Franck A, Guilley H, Jonard G, Richards K and Hirth L. 1980. Nucleotide sequence of cauliflower mosaic virus DNA. Cell 21:285-294.

Frisch D A, Harris-Haller L W, Yokubaitis N T, Thomas T L, Hardin S H and Hall T C. 1995. Complete sequence of the binary vector Bin19. Plant Mol. Biol. 27: 405-409.

Guerineau F and Mullineaux P. 1993. Plant transformation and expression vectors. In Plant Molecular Biology Labfax. Croy RRD (ed). BIOS Scientific Publishers, Blackwell Scientific Publications, pp 121-147.

Hanahan D. 1983. Studies on transformation of Escherichia coli with plasmids. J. Mol. Biol. 166 :557.

Holsters M, Dewaele D, Depicker A, Messenf E, Van Montagu M and Schell J. 1978. Transfection and transformation of Agrobacterium tumefaciens. Mol. Gen. Genet. 136:181-187.

Horsch R B, Fry J E, Hoffinann N L, Eiholtz D, Rogers S G and Fraley R T. 1985. A simple and general method for transferring genes into plants. Science 227:1229-1231.

Ishida Y, Saito H, Ohta S, Hiei Y, Komari T and Kumashiro T. 1996. High efficiency transformation of maize (Zea mays L.) mediated by Agrobacterium tumefaciens. Nature Biotechnology 14:745-750.

Jefferson R A, Burgess S M and Hirsh D. 1986. b-glucuronidase from Escherichia coli as a gene-fusion marker. Proc. Natl. Acad. Sci. (USA) 83:8447-8451.

Kay R, Chan A, Daly M and McPherson J. 1987. Duplication of CaMV 35S promoter sequences creates a strong enhancer for plant genes. Science 236:1299-1302.

Klein T M, Wolf E D, Wu R and Sanford J C. 1987. High velocity microprojectiles for delivering nucleic acids into living cells. Nature 327:70-73.

Komari T, Hiei Y, Yasuhito S, Murai N and Kumashiro T. 1996. Vectors carrying two separate T-DNAs for co-transformation of higher plants mediated by Agrobacterium tumefaciens and segregation of transformants free from selection markers. Plant J. 10:165-174.

McElroy D, Blowers A D, Jenes B and Wu R. 1991. Construction of expression vectors based on the rice actin 1 (Act1) 5′ region for the use in monocot transformation. Mol. Gen. Genet. 231:150-160.

Michael S F. 1994. Mutagenesis by incorporation of a phosphorylated oligofor PCR amplification. BioTechniques 16:411-412.

Vain P, Yean H and Flament P. 1989. Enhancement of production and regeneration of embryogenic type II callus in Zea mays L. by AgNO₃. Plant Cell Tissue and Organ Culture 18:143-151.

1) A clean synthetic vector containing only the elements indispensable to its functionality and to the transgenesis of a cell, said vector comprising, as operationally bound elements indispensable to its functionality and to the transgenesis of a cell:
a nucleic acid sequence coding for a first origin of replication;
a nucleic acid sequence coding for a selection agent; and
a trfA locus coding for a protein that permits an increase in the replication rate of the vector.

2. The clean synthetic vector of claim 1 wherein said cell is a plant cell.

3. The clean synthetic vector of claim 1 wherein said first origin of replication is an RK2 ori.

4. The clean synthetic vector of claim 3 wherein said RK2 ori is a V ori with a broad host range.

5. The clean synthetic vector of claim 1 wherein said selection agent is an antibiotic resistance gene.

6. The clean synthetic vector of claim 5 wherein said antibiotic resistance gene is an npt III gene that confers resistance to kanamycin in bacteria.

7. The clean synthetic vector of claim 1 wherein said trf locus coding for a protein that permits an increase in the replication rate of the vector originates from pRK2.

8. The clean synthetic vector of claim 7 wherein said locus coding for a protein that permits an increase in the replication rate of the vector encodes P285 and P382.

9) The clean synthetic vector of claim 1 wherein said vector comprises the nucleic acid sequence identified by the number SEQ.ID01.

10) The clean synthetic vector of claim 1 wherein said vector is pMRT1105, whose nucleic acid sequence is identified by the number SEQ.ID01.

11) The clean synthetic vector of claim 1 wherein said vector comprises a nucleic acid sequence coding for a second origin of replication

12. The clean synthetic vector of claim 11 wherein said second origin of replication is an E. coli ori.

13. The clean synthetic vector of claim 12 wherein said E. coli ori is a ColEI ori.

14) The clean synthetic vector of claim 1 wherein said vector comprises the nucleic acid sequence identified by the number SEQ.ID02.

15) The clean synthetic vector of claim 1 wherein said vector is plasmid pMRT1106, whose nucleic acid sequence is identified by the number SEQ.ID02.

16) The clean synthetic vector of claim 1 wherein said vector comprises a region comprising a nucleic acid sequence containing a plurality of unique enzyme restriction sites, said plurality collectively called a <<multiple cloning site>> (MCS).

17) The clean synthetic vector of claim 1 wherein said vector comprises a nucleic acid sequence coding for a T-DNA, including a right border, RB, and a left border, LB, which permit the vector to function as a binary plasmid.

18) The clean synthetic vector of claim 16 or claim 17 wherein said MCS is situated near the right border RB of the T-DNA.

19) The clean synthetic vector of claim 17 wherein said vector comprises a nucleic acid sequence coding for at least one expression promoter and at least one transcription terminator situated between the left border, LB, and the right border, RB, of the T-DNA.

20) The clean synthetic vector of claim 19, wherein said expression promoter is chosen from the group consisting of constitutive promoters, inducible promoters and specific promoters.

21) The clean synthetic vector of claim 19 wherein said expression promoter is a plant expression promoter.

22) The clean synthetic vector of claim 21 wherein said expression promoter is chosen from the group consisting of: the 35S CaMV promoter; the ep35S of CaMV; the pea plastocyanin gene promoter, its “enhancer” and derived zones; the “high molecular weight glutenin” (HMWG) promoter of wheat; the CsVMV “Cassava mosaic virus” promoter; the CoYMV “Commelina yellow mosaic virus” promoter; the chimeric promoters of the CsVMV and CoYMV promoters; and derivatives thereof.

23) The clean synthetic vector of claim 19 wherein said expression terminator is chosen from the functional terminators in a plant cell.

24) The clean synthetic vector of claim 23 wherein said functional terminator is a 35S or a nos terminator.

25) The clean synthetic vector of claim 1 wherein said vector comprises a nucleic acid sequence coding for a selection agent that is functional in a plant cell.

26) The clean synthetic vector of claim 25 wherein said nucleic acid sequence coding for a selection agent codes for an antibiotic resistance gene and/or an herbicide resistance gene.

27) The clean synthetic vector of claim 26, wherein said vector comprises a sequence coding for the bar resistance (<<bialaphos resistance>>) or pat (<<phosphinothricin acetyltransferase>>) gene.

28) The clean synthetic vector of claim 25, wherein said nucleic acid sequence coding for a selection agent that is functional in a plant cell is a sequence coding for a mutant or wild-type nptII resistance gene.

29) The clean synthetic vector of claim 25 wherein said nucleic acid sequence coding for a selection agent is situated near the left border of said T-DNA.

30) The clean synthetic vector of claim 1 wherein said vector comprises an expression cassette comprising an expression-promoting nucleic acid sequence operationally bound to a nucleic acid sequence encoding a polypeptide to be expressed, wherein said nucleic acid sequence encoding a polypeptide to be expressed is itself operationally bound to a transcription termination nucleic acid sequence.

31) The clean synthetic vector of claim 30, wherein said polypeptide to be expressed is an enzyme or protein or derivative thereof possessing an activity in vitro and/or in man and/or in animals, said activity selected from a digestive, pancreatic, biliary, antiviral, anti-inflammatory, pulmonary, antimicrobial, nutritional, cosmetic, structural, blood, cardiovascular, ophthalmic, antigenic, immunostimulatory or cerebral activity.

32) A vector of claim 1, said vector being a binary, linear or circular plasmid.

33) A vector of claim 32, said vector chosen from the group consisting of the nucleic acid sequences identified by the numbers SEQ.ID03, SEQ.ID04, SEQ.ID05, SEQ.ID06, SEQ.ID07, SEQ.ID08, SEQ.IDO9, SEQ.ID10, SEQ.ID11, SEQ.ID12, SEQ.ID13, SEQ.ID14, SEQ.ID15, SEQ.ID16, SEQ.ID17, SEQ.ID18, SEQ.ID19, SEQ.ID20, SEQ.ID21 and SEQ.ID22.

34) The clean synthetic vector of claim 1 wherein each functional component can be cleaved independently of the other components.

35) The clean synthetic vector of claim 34, wherein each functional component can be cleaved independently of the other components by enzymatic digestion at a first unique restriction site and a second unique restriction site, both sites being present in one vector.

36) A nucleic acid sequence, said sequence comprising a nucleic acid sequence chosen from the group consisting of the nucleic acid sequences identified by the numbers SEQ.ID01, SEQ.ID02, SEQ.ID03, SEQ.ID04, SEQ.ID05, SEQ.ID06, SEQ.ID07, SEQ.ID08, SEQ.IDO9, SEQ.ID10, SEQ.ID11, SEQ.ID12, SEQ.ID13, SEQ.ID14, SEQ.ID15, SEQ.ID16, SEQ.ID17, SEQ.ID18, SEQ.ID19, SEQ.ID20, SEQ.ID21 and SEQ.ID22.

37) A transgenic plant having stably integrated in its genome a vector of claim 1.

38) A transgenic plant having stably integrated in its genome a nucleic acid sequence of claim 36.

39) The transgenic plant of claim 37 or claim 38 wherein said plant is a dicotyledon species.

40) The transgenic plant of claim 39 wherein said dicotyledon species is selected from the group consisting of potato, tobacco, cotton, lettuce, tomato, melon, cucumber, pea, rape, beetroot and sunflower.

41) The transgenic plant of claim 37 or claim 38 wherein said plant is a monocotyledon species.

42) The transgenic plant of claim 41 wherein said monocotyledon species is selected from the group consisting wheat, barley, oats, rice and maize.

43) A propagule of a transgenic plant of claim 37 or 38.

44) The propagule of claim 43 wherein said propagule is a seed.

45) A cell comprising a vector of claim 1.

46) A cell comprising a nucleic acid sequence selected from the group consisting of those sequences identified by the numbers SEQ.ID01, SEQ.ID02, SEQ.ID03, SEQ.IDO4, SEQ.ID05, SEQ.ID06, SEQ.ID07, SEQ.ID08, SEQ.IDO9, SEQ.ID10, SEQ.ID11, SEQ.ID12, SEQ.ID13, SEQ.ID14, SEQ.ID15, SEQ.ID16, SEQ.ID17, SEQ.ID18, SEQ.ID19, SEQ.ID20, SEQ.ID21 and SEQ.ID22.

47) The cell of claim 45 or claim 46 wherein said cell is a plant cell.

48) A method for the expression of a nucleic acid sequence coding for a polypeptide to be produced in a cell, said method comprising the steps of:
transforming said cell with a vector of claim 1, said vector comprising said nucleic acid sequence coding for said polypeptide; and
maintaining said cell under conditions that permit the expression of said nucleic acid sequence coding for said polypeptide, whereby said polypeptide is produced.

49) A method for the expression of a nucleic acid sequence coding for a polypeptide to be produced in a cell, said method comprising the steps of:
transforming said cell with a nucleic acid sequence of claim 36, said sequence comprising said nucleic acid sequence coding for said polypeptide; and
maintaining said cell under conditions that permit the expression of said nucleic acid sequence coding for said polypeptide, whereby said polypeptide is produced.

50) The method of claim 48 or 49 wherein said cell is a prokaryotic or eukaryotic cell.

51) The method of claim 48 or 49 wherein said cell is chosen from the group consisting of microbial cells, fungal cells, insect cells, animal cells and plant cells.

52) The method of claim 48 or 49 wherein said cell is a plant cell.

53) A method for obtaining a transgenic plant of claim 37, said method comprising the steps of:
a) transforming a plant cell with a vector of claim 1;
b) selecting a plant cell having said vector integrated therein; and
c) propagating the selected plant cell of step (b), either by culturing or by regeneration of chimeric or transgenic whole plants.

54. A method for obtaining a transgenic plant of claim 38, said method comprising the steps of:
a) transforming a plant cell with a nucleic acid of claim 36;
b) selecting a plant cell having said vector integrated therein; and
c) propagating the selected plant cell of step (b), either by culturing or by regeneration of chimeric or transgenic whole plants.


SEQUENCE LISTING (57 sequences)

SEQ ID NO: 1 - DNA, 3508 bp, Artificial Sequence
  Description of Artificial SequencePlasmid pMRT1105
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggaaaagaccgagcgcctttgcgacgctca

SEQ ID NO: 2 - DNA, 4098 bp, Artificial Sequence
  Description of Artificial SequencePlasmid pMRT1106
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggaaaagaccgagcgcctttgcgacgctca

SEQ ID NO: 3 - DNA, 5971 bp, Artificial Sequence
  Description of Artificial SequencePlasmid pMRT1118
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagctttgaattcactcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 4 - DNA, 6016 bp, Artificial Sequence
  Description of Artificial SequencePlasmid pMRT1119
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctagagaattcactcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 5 - DNA, 6017 bp, Artificial Sequence
  Description of Artificial SequencePlasmid pMRT1121
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctagagaattcactcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 6 - DNA, 6016 bp, Artificial Sequence
  Description of Artificial SequencePlasmid pMRT1122
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcttcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctagagaattcactcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 7 - DNA, 6017 bp, Artificial Sequence
  Description of Artificial SequencePlasmid pMRT1155
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcttcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctagagaattcactcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 8 - DNA, 6767 bp, Artificial Sequence
  Description of Artificial SequencePlasmid pMRT1175
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 9 - DNA, 6767 bp, Artificial Sequence
  Description of Artificial SequencePlasmid pMRT1176
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcttcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 10 - DNA, 4805 bp, Artificial Sequence
  Description of Artificial SequencePlasmid pMRT1191
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccgaagcttggccggccgttaacacgcgtggatccttaattaagtcgactctagagaattcactcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 11 - DNA, 8654 bp, Artificial Sequence
  Description of Artificial SequencePlasmid pMRT1192
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggcagttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcttcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctaggggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 12 - DNA, 9143 bp, Artificial Sequence
  Description of Artificial SequencePlasmid pMRT1193
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctagagaattcccagctttgagtggccgtagatttgcaaaagcaatggctaacagacacatattctgccaaaccccaagaaggataatcacttttcttagataaaaaagaacagaccaatatacaaacatccacacttctgcaaacaatacatcagaactaggattacgccgattacgtggctttagcagactgtccaaaaatctgttttgcaaagctccaattgctccttgcttatccagcttcttttgtgttggcaaactgcgcttttccaaccgattttgttcttctcgcgctttcttcttaggctaaacaaacctcaccgtgcacgcagccatggtcctgaaccttcacctcgtccctataaaagcctagccaaccttcacaatcttatcatcacccacaacaccgagcaccacaaactagagatccccggggatccacgcgtcttaaggcggccgcggtaccgggcccgtcgactctagagcggccgccaccgcggtggagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagcttcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 13 - DNA, 6865 bp, Artificial Sequence
  Description of Artificial SequencePlasmid pMRT1195
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggccctagaactagtggatcccccgggtcatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatccccgggctgcaggaattcgatatcaagcttatcgtctacctacaaaaaagctccgcacgaggctgcatttgtcacaaatcatgaaaagaaaaactaccgatgaacaatgctgagggattcaaattctacccacaaaaagaagaaagaaagatctagcacatctaagcctgacgaagcagcagaaatatataaaaatataaaccatagtgcccttttcccctcttcctgatcttgtttagcatggcggaaattttaaaccccccatcatctcccccaacaacggcggatcgcagatctacatccgagagccccattccccgcgagatccgggccggatccacgccggcgagagccccagccgcgagatcccgcccctcccgcgcaccgatctgggcgcgcacgaagccgcctctcgcccacccaaactaccaaggccaaagatcgagaccgagacggaaaaaaaaaacggagaaagaaagaggagaggggcggggtggttaccggcgcggcggcggcggagggggaggggggaggagctcgtcgtccggcagcgaggggggaggaggtggaggtggtggtggtggtggtggtagggttggggggatgggaggagaggggggggtatgtatatagtggcgatggggggcgtttctttggaagcggagggagggccggcctcgtcgctggctcgcgatcctcctcgcgtttccggcccccacgacccggacccacctgctgtttttccttttcctttttttccttccttttttttttttgggcagcgagacgtgcggtgcgtgcggacaactcacggtgatagtgggggggtgtggagactattgtccagttggctggactggggtgggttgggttgggttgggttgggctgggcttgctatggatcgtggatagcactttgggctttaggaactttaggggttgtttttgtaaatgtttggagtctaagtttatcttttatttttactagaaaaaatacccatgcgctgcaacgggggaaagctattttaatcttattattgttcattgtgagaattcgcctgaatatatatttttctcaaaaattatgtcaaattagcatatgggtttttttaaagatatttcttatacaaatccctctgtatttacaaaagcaaacgaacttaaaacccgactcaaatacagatatgcatttccaaaagcgaataaacttaaaaaccaattcatacaaaaatgacgtatcaaagtaccgacaaaaacatcctcaatttttataatagtagaaaagagtaaatttcactttgggccaccttttattaccgatattttactttataccaccttttaactgatgttttcacttttgaccaggtaatcttacctttgttttattttggactatcccgactctcttctcaagcatatgaatgactcgacctgcaggcatgcaagcttcagctgctcgatcgaggtctagcgaagcttggccggccgttaacacgcgtggatccttaattaagtcgactctagagaattcactcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 14 - DNA, 8654 bp, Artificial Sequence
  Description of Artificial SequencePlasmid pMRT1196
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctaggggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 15 - DNA, 7943 bp, Artificial Sequence
  Description of Artificial SequencePlasmid pMRT1201
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccgaagcttggccggccgttaacacgcgtggatccttaattaagtcgactctagagaattaattcccagctttgagtggccgtagatttgcaaaagcaatggctaacagacacatattctgccaaaccccaagaaggataatcacttttcttagataaaaaagaacagaccaatatacaaacatccacacttctgcaaacaatacatcagaactaggattacgccgattacgtggctttagcagactgtccaaaaatctgttttgcaaagctccaattgctccttgcttatccagcttcttttgtgttggcaaactgcgcttttccaaccgattttgttcttctcgcgctttcttcttaggctaaacaaacctcaccgtgcacgcagccatggtcctgaaccttcacctcgtccctataaaagcctagccaaccttcacaatcttatcatcacccacaacaccgagcaccacaaactagagatccccggggatccacgcgtcttaaggcggccgcggtaccgggcccgtcgactctagagcggccgccaccgcggtggagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcactcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 16 - DNA, 5614 bp, Artificial Sequence
  Description of Artificial SequencePlasmid pMRT1202
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggccctagaactagtggatcccccgggtcatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatccccgggcccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctagagaattcactcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 17 - DNA, 7503 bp, Artificial Sequence
  Description of Artificial SequencePlasmid pMRT1203
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcttcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccctactccaagaatatcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttggccggccgttaacacgcgtggatccttaattaagtcgactctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 18 - DNA, 9390 bp, Artificial Sequence
  Description of Artificial SequencePlasmid pMRT1204
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggcagttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcttcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccctactccaagaatatcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttggccggccgttaacacgcgtggatccttaattaagtcgactctaggggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 19 - DNA, 7503 bp, Artificial Sequence
  Description of Artificial SequencePlasmid pMRT1205
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccctactccaagaatatcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttggccggccgttaacacgcgtggatccttaattaagtcgactctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 20 - DNA, 9390 bp, Artificial Sequence
  Description of Artificial SequencePlasmid pMRT1206
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccctactccaagaatatcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttggccggccgttaacacgcgtggatccttaattaagtcgactctaggggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 21 - DNA, 10003 bp, Artificial Sequence
  Description of Artificial SequencePlasmid pMRT1210
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccgaagcttggccggccgttctagaactagtggatcccccgggtcatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatccccgggctgcaggaattcgatatcaagcttatcgtctacctacaaaaaagctccgcacgaggctgcatttgtcacaaatcatgaaaagaaaaactaccgatgaacaatgctgagggattcaaattctacccacaaaaagaagaaagaaagatctagcacatctaagcctgacgaagcagcagaaatatataaaaatataaaccatagtgcccttttcccctcttcctgatcttgtttagcatggcggaaattttaaaccccccatcatctcccccaacaacggcggatcgcagatctacatccgagagccccattccccgcgagatccgggccggatccacgccggcgagagccccagccgcgagatcccgcccctcccgcgcaccgatctgggcgcgcacgaagccgcctctcgcccacccaaactaccaaggccaaagatcgagaccgagacggaaaaaaaaaacggagaaagaaagaggagaggggcggggtggttaccggcgcggcggcggcggagggggaggggggaggagctcgtcgtccggcagcgaggggggaggaggtggaggtggtggtggtggtggtggtagggttggggggatgggaggagaggggggggtatgtatatagtggcgatggggggcgtttctttggaagcggagggagggccggcctcgtcgctggctcgcgatcctcctcgcgtttccggcccccacgacccggacccacctgctgtttttccttttcctttttttccttccttttttttttttgggcagcgagacgtgcggtgcgtgcggacaactcacggtgatagtgggggggtgtggagactattgtccagttggctggactggggtgggttgggttgggttgggttgggctgggcttgctatggatcgtggatagcactttgggctttaggaactttaggggttgtttttgtaaatgtttggagtctaagtttatcttttatttttactagaaaaaatacccatgcgctgcaacgggggaaagctattttaatcttattattgttcattgtgagaattcgcctgaatatatatttttctcaaaaattatgtcaaattagcatatgggtttttttaaagatatttcttatacaaatccctctgtatttacaaaagcaaacgaacttaaaacccgactcaaatacagatatgcatttccaaaagcgaataaacttaaaaaccaattcatacaaaaatgacgtatcaaagtaccgacaaaaacatcctcaatttttataatagtagaaaagagtaaatttcactttgggccaccttttattaccgatattttactttataccaccttttaactgatgttttcacttttgaccaggtaatcttacctttgttttattttggactatcccgactctcttctcaagcatatgaatgactcgacctgcaggcatgcaagcttcagctgctcgatcgaggtctagaacacgcgtggatccttaattaagtcgactctagagaattaattcccagctttgagtggccgtagatttgcaaaagcaatggctaacagacacatattctgccaaaccccaagaaggataatcacttttcttagataaaaaagaacagaccaatatacaaacatccacacttctgcaaacaatacatcagaactaggattacgccgattacgtggctttagcagactgtccaaaaatctgttttgcaaagctccaattgctccttgcttatccagcttcttttgtgttggcaaactgcgcttttccaaccgattttgttcttctcgcgctttcttcttaggctaaacaaacctcaccgtgcacgcagccatggtcctgaaccttcacctcgtccctataaaagcctagccaaccttcacaatcttatcatcacccacaacaccgagcaccacaaactagagatccccggggatccacgcgtcttaaggcggccgcggtaccgggcccgtcgactctagagcggccgccaccgcggtggagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcactcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 22 - DNA, 8987 bp, Artificial Sequence
  Description of Artificial SequencePlasmid pMRT1212
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggccctagaactagtggatcccccgggtcatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatccccgggcccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccctactccaagaatatcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttggccggccgttaacacgcgtggatccttaattaagtcgactctaggggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 23 - DNA, 28 bp, Artificial Sequence
  Description of Artificial SequenceOligo desoxynucleotide containing AvrII restriction site
aacctaggaaaagaccgagcgcctttgc

SEQ ID NO: 24 - DNA, 43 bp, Artificial Sequence
  Description of Artificial SequenceOligo Desoxynucloetide containing StuI restriction site
cggattaatggtagaaggcctttcacgggagggttcgagaagg

SEQ ID NO: 25 - DNA, 43 bp, Artificial Sequence
  Description of Artificial SequenceOligodesoxynucleotide containing StuI restriction site
tgaaaggccttctaccattaatccgcgataaacccagcgaacc

SEQ ID NO: 26 - DNA, 52 bp, Artificial Sequence
  Description of Artificial SequenceOligodesoxynucleotide containing BstXI restriction site
atgcatccaaaattttggtagaatttacaagctataaggttattgtcctggg

SEQ ID NO: 27 - DNA, 29 bp, Artificial Sequence
  Description of Artificial SequenceOligodesoxynucleotide situated upstream of NdeI restriction site
atcgacgaggaaatcgtcgtgctgtttgc

SEQ ID NO: 28 - DNA, 30 bp, Artificial Sequence
  Description of Artificial SequenceOligodesoxynucleotide containing AvrII restriction site
aaacctaggaaatgccagtaaagcgctggc

SEQ ID NO: 29 - DNA, 38 bp, Artificial Sequence
  Description of Artificial SequenceOligodesoxynucleotide containing AvrII and AatII restriction sites
ttcctaggttgacgtcttctgatgggctgcctgtatcg

SEQ ID NO: 30 - DNA, 47 bp, Artificial Sequence
  Description of Artificial SequenceOligodesoxynucleotide containing SmaI restriction site
cctatggatatcccccgggggatagccccagtacattaaaaacgtcc

SEQ ID NO: 31 - DNA, 48 bp, Artificial Sequence
  Description of Artificial SequenceOligodesoxynucleotide containing restriction site SmaI
ctatcccccgggggatatccataggcccgatctagtaacatagatgac

SEQ ID NO: 32 - DNA, 47 bp, Artificial Sequence
  Description of Artificial SequenceOligodesoxynucleotide containing Bsp120I restriction site
gcgcacttgggcccatagctcgacgaacgatcgttcaaacatttggc

SEQ ID NO: 33 - DNA, 47 bp, Artificial Sequence
  Description of Artificial SequenceOligodesoxynucleotide containing Bsp120I restriction site
ttcgtcgagctatgggcccaagtgcgcatcccgtgggcgaagaactc

SEQ ID NO: 34 - DNA, 25 bp, Artificial Sequence
  Description of Artificial SequenceOligodesoxynucleotide downstream of a BstBI restriction site
ttcttgacgagttcttctgagcggg

SEQ ID NO: 35 - DNA, 47 bp, Artificial Sequence
  Description of Artificial SequenceOligodesoxynucleotide containing restriction sites KpnI, HindIII, EcoRI and XhoI
cggtaccgaagctttgaattcactcgagcagattgtcgtttcccgcc

SEQ ID NO: 36 - DNA, 36 bp, Artificial Sequence
  Description of Artificial SequenceOligodesoxynucleotide containing restriction sites AvrII and AgeI
tatcctaggaaccggtaaaccctgtggttggcatgc

SEQ ID NO: 37 - DNA, 29 bp, Artificial Sequence
  Description of Artificial SequenceOligodesoxynucleotide used for synthesis of fragment “MCS-beg. Pnos”
atatgagactctaattggataccgagggg

SEQ ID NO: 38 - DNA, 49 bp, Artificial Sequence
  Description of Artificial SequenceOligodesoxynucleotide containing XhoI, EcoRI, HindIII et KpnI restriction sites
gctcgagtgaattcaaagcttcggtaccgttgaaggagccactcagccg

SEQ ID NO: 39 - DNA, 25 bp, Artificial Sequence
  Description of Artificial SequenceOligodesoxynucleotide for synthesis of part of npt II and BspEI site
ggaatcgaaatctcgtgatggcagg

SEQ ID NO: 40 - DNA, 24 bp, Artificial Sequence
  Description of Artificial SequenceOligodesoxynucleotide used for synthesis of part of Pnos and npt II
attattgcgcgttcaaaagtcgcc

SEQ ID NO: 41 - DNA, 52 bp, Artificial Sequence
  Description of Artificial SequenceOligodesoxynucleotide used in the synthesis of XbaI, SalI, PacI, BamHI, MluI, HpaI et FseI restriction sites of an MCS
agcttggccggccgttaacacgcgtggatccttaattaagtcgactctagag

SEQ ID NO: 42 - DNA, 52 bp, Artificial Sequence
  Description of Artificial SequenceOligodesoxynucleotide used in the synthesis of XbaI, SalI, PacI, BamHI, MluI, HpaI et FseI restriction sites of an MCS
aattctctagagtcgacttaattaaggatccacgcgtgttaacggccggcca

SEQ ID NO: 43 - DNA, 30 bp, Artificial Sequence
  Description of Artificial SequenceOligodesoxynucleotide containing BspEI restriction site
taatctgcatccggatctggatcgtttcgc

SEQ ID NO: 44 - DNA, 30 bp, Artificial Sequence
  Description of Artificial SequenceOligodesoxynucleotide containing BspEI restriction site
acgatccagatccggatgcagattatttgg

SEQ ID NO: 45 - DNA, 30 bp, Artificial Sequence
  Description of Artificial SequenceOligodesoxynucleotide containing BglII restriction site
atgggtcacgacgagatcttcgccgtcggg

SEQ ID NO: 46 - DNA, 19 bp, Artificial Sequence
  Description of Artificial SequenceOligodesoxynucleotide containing BstBI restriction site
aggcattggtttcgaagcg

SEQ ID NO: 47 - DNA, 21 bp, Artificial Sequence
  Description of Artificial SequenceOligodesoxynucleotide used for synthesis of plasmid pUC19-uidA-Tnos delta EcoRI
tacgccaagcttggcaattcc

SEQ ID NO: 48 - DNA, 29 bp, Artificial Sequence
  Description of Artificial SequenceOligodesoxynucleotide containing NcoI and SmaI restriction sites
aatacccgggaccatggtccgtcctgtag

SEQ ID NO: 49 - DNA, 24 bp, Artificial Sequence
  Description of Artificial SequenceOligodesoxynucleotide situated upstream of SnaBI restriction site
atagtctgccagttcagttcgttg

SEQ ID NO: 50 - DNA, 9688 bp, Artificial Sequence
  Description of Artificial SequencepMRT1334
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttcctactccaagaatatcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttggccggccgttaacacgcgtggatccttaattaagtcgactctaggggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 51 - DNA, 15208 bp, Artificial Sequence
  Description of Artificial SequencepMRT1335
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaatttcgagatatcgcatgcgatccccgtcaccggtgtgagggaactagttttgatcttgaaagatcttttatctttagagttaagaactctttcgtattttggtgaggttttatcctcttgagttttggtcatagacctattcatggctctgataccaatttttaagcgggggcttatgcggattatttcttaaattgataaggggttattagggggtatagggtataaatacaagcattcccttagcgtatagtataagtatagtagcgtacctctatcaaatttccatcttcttaccttgcacagggcctgcaaccttatccttccttgtcttcctccttccttccgtccacttcatcatatttaaaccaaacctacgggggagtcaacgtaaccaaccctgccttagcatcttttccctaacggcctcctgcctaagcggtacttctagcttcgaacggcgtctgggctccaggtttagtcgtctcgtgtctggtttatattcacgacaaagatctatagggactttaggagatctggattttagtactggattttggttttaggaattagaaattttattgatagaagtattttacaaatacaaatacatactaagggtttcttatatgctcaacacatgagcgaaaccctataagaaccctaatttcccttatcgggaaactactcacacattatttatggagaaaatagagagagatagatttgtagagagagactggtgatttcagcgtaccgaattctctagcggtagcaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacgtaacataagggactgaccacccctagagtcgacttaattaaggatccacgcgtgttaacggccggccaagcttgggctgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattaccctttgttgaaaagtctcaatagccctttggtcttctgagactgtatctttgacatttttggagtaggggtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattaccctttgttgaaaagtctcaatagccctttggtcttctgagactgtatctttgatattcttggagtagggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 52 - DNA, 9285 bp, Artificial Sequence
  Description of Artificial SequencepMRT1336
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccgaagcttggccggccgttaacacgcgtggatccttaattaagtcgactctagagctgcctgcagcactagtatccgccgtcatcaatgacatcatcacagtactgaggagatgaatagctagccatgacactctgtgcgaatattgaagacgtaagcactgacgacaacaatgaaaagaagaagataaggtcggtgattgtgaaagagacatagaggacacatgtaaggtggaaaatgtaagggcggaaagtaaccttatgcatttgtaatttggttacgactagtgattgatgtgatatcaagattgatgtgatatctccactgacgtaagggatgacgcatgccacgactagtgattgatgtgatatcaagattgatgtgatatctccactgacgtaagggatgacgcatgccacgactagtgattgatgtgatatcaagattgatgtgatatctccactgacgtaagggatgacgcatgccacgactagtgattgatgtgatatcaagattgatgtgatatctccactgacgtaagggatgacgcatgccacgttacccggtatgccggttcccaagctttatttccttatttaagcacttgtgtagtagcttagaaaaccaacacaacaacctagaggccgaattaacacgcgtggatccttaattaagtcgactctaggggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 53 - DNA, 31 bp, Artificial Sequence
  Description of Artificial SequenceOligodesoxynucleotide
agctctagagctgcctgcagcactagtatcc

SEQ ID NO: 54 - DNA, 29 bp, Artificial Sequence
  Description of Artificial SequenceOligodesoxynucleotide
cggaattcggcctctaggttgttgtgttg

SEQ ID NO: 55 - DNA, 8289 bp, Artificial Sequence
  Description of Artificial SequencepMRT1337
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggaaaggtatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccttctaccataatccgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatacatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctaccaaaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcctaggttgacgtcttctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggctatcccccgggggatatccataggcccgatctagtaacataatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgttcgtcgagctatgggcccaagttgcgcatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggatgcagattatttggattgagagtaaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacggtaccctactccaagaatatcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttggccggccgttaacacgcgtggatccaaggagatataacaatgaagactaatctttttctctttctcatcttttcacttctcctatcattatcctcggccgaattcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaacatgatgagctttaagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggtttaccggttcctaggaaaagaccgagcgcctttgcgacgctca

SEQ ID NO: 56 - DNA, 14108 bp, Artificial Sequence
  Description of Artificial SequencepMRT1341
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaatttcgagatatcgcatgcgatccccgtcaaccggtgtgagggaactagttttgatcttgaaagatcttttatctttagagttaagaactctttcgtattttggtgaggttttatcctcttgagttttggtcatagacctattcatggctctgataccaatttttaagcgggggcttatgcggattatttcttaaattgataaggggttattagggggtatagggtataaatacaagcattcccttagcgtatagtataagtatagtagcgtacctctatcaaatttccatcttcttaccttgcacagggcctgcaaccttatccttccttgtcttcctccttccttccgtccacttcatcatatttaaaccaaacctacgggggagtcaacgtaaccaaccctgccttagcatcttttccctaacggcctcctgcctaagcggtacttctagcttcgaacggcgtctgggctccaggtttagtcgtctcgtgtctggtttatattcacgacaaagatctatagggactttaggagatctggattttagtactggattttggttttaggaattagaaattttattgatagaagtattttacaaatacaaatacatactaagggtttcttatatgctcaacacatgagcgaaaccctataagaaccctaatttcccttatcgggaaactactcacacattatttatggagaaaatagagagagatagatttgtagagagagactggtgatttcagcgtaccgaattcttaaagctcatcatgtttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatgatcagcgagttgcacgccgccgtcttcgatgttgtggcgggtcttgaagttggctttgatgccgttcttttgcttgtcggccatgatgtatacgttgtgggagttgtagttgtattccaacttgtggccgaggatgtttccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactgaattcggccgaggataatgataggagaagtgaaaagatgagaaagagaaaaagattagtcttcattgttatatctccttggatccacgcgtgttaacggccggccaagcttgggctgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattaccctttgttgaaaagtctcaatagccctttggtcttctgagactgtatctttgacatttttggagtaggggtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattaccctttgttgaaaagtctcaatagccctttggtcttctgagactgtatctttgatattcttggagtagggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 57 - DNA, 15077 bp, Artificial Sequence
  Description of Artificial SequencepMRT1342
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggtaccgaagcttggccggccgttaacacgcgtggatccttaattaagtcgactctagagctgcctgcagcactagtatccgccgtcatcaatgacatcatcacagtactgaggagatgaatagctagccatgacactctgtgcgaatattgaagacgtaagcactgacgacaacaatgaaaagaagaagataaggtcggtgattgtgaaagagacatagaggacacatgtaaggtggaaaatgtaagggcggaaagtaaccttatgcatttgtaatttggttacgactagtgattgatgtgatatcaagattgatgtgatatctccactgacgtaagggatgacgcatgccacgactagtgattgatgtgatatcaagattgatgtgatatctccactgacgtaagggatgacgcatgccacgactagtgattgatgtgatatcaagattgatgtgatatctccactgacgtaagggatgacgcatgccacgactagtgattgatgtgatatcaagattgatgtgatatctccactgacgtaagggatgacgcatgccacgttacccggtatgccggttcccaagctttatttccttatttaagcacttgtgtagtagcttagaaaaccaacacaacaacctagaggccgaattaacacgcgtggatccttaattaagtcgactctaggggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgctagagaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtcggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca